SEQUENCE LISTING (10 sequences)

SEQ ID NO: 1 - DNA, 20 bp, Artificial Sequence
  synthetic primer
ctgctctgcctctagcacac

SEQ ID NO: 2 - DNA, 20 bp, Artificial Sequence
  synthetic primer
ttaagggtagcacatgtctg

SEQ ID NO: 3 - DNA, 20 bp, Artificial Sequence
  synthetic primer
acactggtgagaagccattc

SEQ ID NO: 4 - DNA, 20 bp, Artificial Sequence
  synthetic primer
agttctatggctcgaagcag

SEQ ID NO: 5 - DNA, 20 bp, Artificial Sequence
  synthetic primer
ctgctctgcctctagcacac

SEQ ID NO: 6 - DNA, 20 bp, Artificial Sequence
  synthetic primer
ttaagggtagcacatgtctg

SEQ ID NO: 7 - DNA, 20 bp, Artificial Sequence
  synthetic primer
acactggtgagaagccattc

SEQ ID NO: 8 - DNA, 20 bp, Artificial Sequence
  synthetic primer
agttctatggctcgaagcag

SEQ ID NO: 9 - DNA, 20 bp, Artificial Sequence
  synthetic primer
cacgtggaaatcaacgggat

SEQ ID NO: 10 - DNA, 20 bp, Artificial Sequence
  synthetic primer
gcgcacaatcatgttggaca

(19) United States
(12) Patent Application Publication
(10) Pub. No.: US 2006/0040253 A1
(43) Pub. Date: Feb. 23, 2006

(54) Use of Smad3 inhibitor in the treatment of fibrosis dependent on epithelial to mesenchymal transition as in the eye and kidney

(76) Inventors: Anita Roberts, Bethesda, MD (US); Shizuya Saika, Wakayama (JP); Akira Ooshima, Bethesda, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/183,194

(22) Filed: Jul. 15, 2005

Related U.S. Application Data
(63) Continuation of application No. PCT/US04/03563, filed on Jan. 16, 2004.
(60) Provisional application No. 60/441,297, filed on Jan. 17, 2003.
(60) Provisional application No. 60/508,671, filed on Oct. 3, 2003.
(60) Provisional application No. 60/534,500, filed on Jan. 6, 2004.

(51) Int. Cl. A61K 48/00; C12Q 1/00

(52) U.S. Cl. 435/4; 514/44

(57) ABSTRACT

The invention is related to inhibition of Smad3 to ameliorate Smad3 mediated epithelial to mesenchymal transition.

### RELATED APPLICATIONS

This application is a continuation of International Patent application No.: PCT/US 2004/003563, filed Jan. 16, 2004, designating the U.S. and published in English as WO 2004/064770 on Aug. 5, 2004, which claims the benefit of U.S. Provisional Application No. 60/441,297 filed Jan. 17, 2003, U.S. Provisional Application No. 60/508,671 filed Oct. 3, 2003, and U.S. Provisional patent application No. 60/534,500 filed Jan. 6, 2004, all of which are hereby expressly incorporated by reference in their entireties.

### FIELD OF THE INVENTION

The invention is related to inhibition of Smad3 to ameliorate Smad3 mediated epithelial to mesenchymal transition.

### BACKGROUND OF THE INVENTION

Basic features of the Smad signaling pathway downstream of TGF-β/activin receptors are as follows. Upon ligand binding, receptor-activated Smads2/3 are phosphorylated by the type I receptors, form a heteromeric complex with Smad4, and translocate to the nucleus where they regulate target gene expression both by direct DNA binding and through interaction with other transcription factors, coactivators, and corepressors.

### SUMMARY OF THE INVENTION

The invention is related to inhibition of Smad3 to ameliorate Smad3 mediated epithelial to mesenchymal transition and fibrotic sequelae of the event.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Epithelial-mesenchymal transition of primary lens epithelial cells in vitro is dependent on endogenous TGF-β. EMT in primary porcine lens epithelial cells as evidenced by staining for αSMA at 48 hr-culture (a) is blocked by addition of a neutralizing pan-specific antibody against TGF-β (20 μg/ml) (b). Indirect immunostaining by diaminobenzidine color reaction methylgreen counterstaining, bar, 50 μm. Panel c indicates the percentage of αSMA-positive cells in cultures shown in Panels a and b. Panel d indicates the protein expression level of αSMA as determined by Western blot analysis of lysates of porcine lens epithelial cells cultured in serum-free medium in the presence of either non-immune IgG or anti-TGF-β neutralizing antibody for 72 hrs. Actin serves as a loading control.

FIG. 2. Histology of lens epithelial cells post-capsular injury in Smad3-knockout mice. Hematoxylin and eosin-stained paraffin sections of WT (Smad3^{+/+}, left panels) and KO (Smad3^{ex8/ex8}, right panels) uninjured murine globes (a, b) or of eyes at day 5 (c, d), or week 8 (e, f) post-injury. Cells in WT injured lenses are of a fibroblastic appearance, but not in KO lenses. The appearance of KO cells at week 8 is similar to that of normal lens epithelial cells (arrows, f). Arrows and AC indicate lens epithelial cells and anterior lens capsule, respectively. Bar, 50 μm.

FIG. 3. Smad3 is required for expression of snail mRNA in lens epithelial cells in response to injury. Expression of snail mRNA in WT (Smad3^{+/+}, left panels and g, h, i) and KO (Smad3^{ex8/ex8}, right panels) lens epithelium at day 1 (a, b), day 3 (c, d), or week 2 post-injury (e-i). Panels g and h area high magnification pictures of the boxed areas in panel e. Panel i: sense probe in serial section from panel h. Snail mRNA is detected in epithelial cells of WT injured lenses, but not in KO injured lenses. Filled arrows in panels a, c, and h indicate snail mRNA-expressing cells. Open arrows indicate the margin of the capsular break made by puncture injury; AC, anterior capsule. Bar, 50 μm (a-f), 12 μm (g-i).

FIG. 4. Smad3 is required for expression of αSMA protein following lens injury. Panels a and b, c and d, or e and f indicate injured anterior lens tissues at day 5, week 1 or 2, respectively. Arrows indicate αSMA-expressing cells in WT mice (a, c, and e) and asterisks indicate non-expressing cells in KO mice (b, d, and f). The dotted line with AC indicates broken anterior capsules. WT (Smad3^{+/+}, left panels); KO (Smad3^{ex8/ex8}, right panels). Bar, 50 μm.

FIG. 5. Extracellular matrix components, lumican and collagen type I, are expressed in epithelial cells of injured WT lenses, but not in injured KO lenses. Immunohistochemical staining for lumican (panels a, b) or type I collagen (panels c, d) at indicated times post-injury in eyes of WT (Smad3^{+/+}, left panels) and KO (Smad3^{ex8/ex8}, right panels) mice. Arrow indicates deposition of lumican (panel a, at Day 5) or collagen I (panel c, at Week 8). Asterisk in panel b or d indicates cells in injured KO lenses without immunoreactivity for lumican or collagen I, respectively. The dotted line with AC indicates broken anterior capsules. Indirect immunostaining, bar 50 μm.

FIG. 6. TGF-β1 and TGF-β2 are differentially expressed in lens epithelium following injury. Immunohistochemical staining for TGF-β1 (panels a-f) or TGF-β2 (panels g-n), at indicated times post-injury in eyes of WT (Smad3^{+/+}, left panels) and KO (Smad3^{ex8/ex8}, right panels) mice. TGF-β1 protein is not detected in uninjured epithelium of WT and KO mice, but is up-regulated in WT epithelium following injury (c, e), but not in KO mice (d, f). TGF-β2 protein is observed in peripheral epithelium (h, j), but not in central epithelium (g, i) of both WT and KO mice. Post-injury, both mesenchymal-like cells in WT (k, m) and epithelial cells in KO (l, n) around the capsular break are labeled with anti-TGF-β2 antibody. AC, anterior capsule. Indirect immunostaining, bar 50 μm.

FIG. 7. Smad3 is required for expression of αSMA in outgrowths of mouse lens epithelial cells. Smad3^{+/+} (WT) lens epithelial cells (a, star) migrate out of capsular specimens placed in chamber slides, whereas the outgrowth of Smad3^{-/-} (KO) cells (b, asterisks) is comparatively less. WT cells located at the edge of the migrating epithelial sheet (a, arrows) exhibited more of a fibroblast-like morphology compared to KO cells. Immunofluorescence staining for αSMA identified a small number of WT cells located at the migrating edge (c), whereas no labeled cells were seen in cultures of KO specimens (d). Western blotting also showed expression of αSMA in WT, but not KO cells at Day 6 and 12 of culture (e). Panel f indicates the mean value of the maximal length of cell outgrowth in each specimen with the number of specimens in each genotype shown in parentheses. Capsule, lens capsular explant; dotted line, margin of the capsule; Fiber debris, lens fiber contamination. Bar, 50 μm.

FIG. 8. Induction of EMT by TGF-β2 in organ-cultured lenses requires Smad3. Lenses were cultured in serum-free Dulbecco's modified Eagle's medium supplemented with antibiotics in the presence or absence of 10 ng/ml of TGF-β2 as indicated. Sections were stained with hematoxylin/eosin (a, b, e, f), antibodies to lumican (c, d), antibodies to αSMA (g, h), or antibodies to type I collagen (i, j) at the indicated times. Lumican is expressed at Day 5 and αSMA and collagen I are expressed at Day 10 in WT lenses, but not in KO lenses. Bar, 50 μm. WT (Smad3^{+/+}) lenses (left panels); KO (Smad3^{ex8/ex8}) lenses (right panels).

FIG. 9. Smad3 is necessary for both EMT of lens epithelial cells and for subsequent elaboration of ECM proteins by myofibroblasts. Data indicate that injury-induced EMT of lens epithelial cells is initiated by activation of TGF-β2 and mediated by Smad3-dependent expression of the early marker, snail, followed by expression of lumican to enhance EMT of lens epithelium, and finally markers of the myofibroblast, αSMA, and of the fibrotic phenotype, collagen I. While loss of Smad3 blocks the process at the level of EMT, previous studies implicate Smad3 in elaboration of ECM by mesenchymal cells. (Verrecchia F and Mauviel A. 2002 J Invest Dermatol 118:211-215)

FIG. 10. Smad3-null mice maintain the renal architecture and reverse epithelial-mesenchymal transition. (a) Obstructed kidneys from wild-type (WT) and Smad3-null (KO) mice at day 14 after unilateral ureteral obstruction (UUO). (b and c) Haematoxylin-eosin staining of the obstructed kidneys at day 14 after UUO in WT (b) and KO (c) mice. Scale bar, 20 μm. (d-g) Dual immunofluorescence of E-cadherin (green) and α-smooth muscle actin (red) in obstructed kidneys of WT (d and e) and KO (f and g) mice at day 7 (d and f) and 14 (e and g) after UUO. DAPI (blue) was used for nuclear staining. Scale bar, 20 μm. (h) Immunoblot of E-cadherin (E-cad) and α-smooth muscle actin (α-SMA) with extracted proteins from kidneys of WT and KO mice with UUO and sham-operated WT (Sham). (i) Northern blot of Snail mRNA in kidneys of WT and KO mice with UUO and sham-operated (Sham) counterparts.

FIG. 11. In situ hybridization of α-smooth muscle actin, Snail and TGF-β1. (a-d) De novo expression of Snail (a) and α-smooth muscle actin (α-SMA) (c) mRNAs in the renal tubular epithelial cells of wild-type (WT) mice at day 7 after UUO. No positive signals for Snail (b) or α-SMA (d) mRNA in Smad3-null (KO) counterparts. (e and f) Signals for TGF-β1 mRNA in WT (e) and KO mice (f) at day 14 after UUO (e). Insets, negative controls reacted with sense probe. Counterstained in nuclear fast red solution. Scale bar, 20 μm. Similar results were obtained from three additional experiments.

FIG. 12. Lack of Smad3 prevents renal fibrosis, monocyte influx and TGF-β1 upregulation. (a and b) Immunofluorescence of type I collagen in obstructed kidneys of wild-type (WT) (a) and Smad3-null (KO) (b) mice at day 14 after UUO. (c) Hydroxyproline content in obstructed kidneys from WT and KO mice and sham-operated (Sham) WT. (d and e) Immunofluorescence of F4/80 antigen, a mouse monocyte marker, in obstructed kidneys from WT (d) and KO (e) mice at day 14 after UUO. DAPI (blue) was used for nuclear staining. Scale bar, 20 μm. (f) Number of monocytes per unit area in obstructed kidneys from WT and KO mice with UUO and sham-operated (Sham) WT. (g) Northern blot of TGF-β1 mRNA in kidneys from WT and KO mice with UUO and sham-operated (Sham) counterparts. (h) Active and total TGF-β1 concentrations as determined by immunoassay in kidneys of WT and KO mice and sham-operated (Sham) WT. Results are means±standard deviation of 4 to 5 samples. *P<0.01 compared with Sham or KO.

FIG. 13. Smad3-mediated epithelial-mesenchymal transition in cultured renal tubular epithelial cells. (a-d) Phase-contrast microscopy of the epithelial cells from wild-type (WT) (a and b) and Smad3-null (KO) (c and d) mice in the absence (a and c) or presence (b and d) of TGF-β1 (10 ng/ml) for 24 h. Scale bar, 100 μm. (e-h) Dual immunofluorescence of E-cadherin (green) and α-smooth muscle cell actin (red) in the epithelial cells from WT (e and f) and KO (g and h) mice in the absence (e and g) or presence (f and h) of TGF-β1 (10 ng/ml) for 24 h. Scale bar, 20 μm. (i) Immunoblot of E-cadherin (E-cad) and α-smooth muscle actin (α-SMA) with extracted protein from epithelial cells of WT and KO mice in the absence (−) or presence (+) of TGF-β1 (10 ng/ml) for 24 h. (j) Northern blot of Snail mRNA in the epithelial cells from WT and KO mice in the absence (−) or presence (+) of TGF-β1 (10 ng/ml) for 8 h. Similar results were obtained from three additional experiments.

FIG. 14. Smad3-mediated autoinduction of TGF-β1 in cultured renal tubular epithelial cells. (a) Concentration of total TGF-β1 in culture medium of renal tubular epithelial cells from wild-type (WT) and Smad3-null (KO) mice. Results are means±standard deviation of 4 to 5 samples. *P<0.05 as compared with KO. (b) Northern blot of TGF-β1 mRNA in epithelial cells from WT and KO mice in the absence (−) or presence (+) of TGF-β1 (10 ng/ml) for 24 h. Cells without TGF-β1 were further treated with a neutralizing antibody against TGF-β₁ (20 μg/ml) to exclude any effects of endogenous TGF-β1. The same amount of normal IgG was added to the medium of TGF-β1-treated cells. Results are means±standard deviation of 4 samples. *P<0.01 as compared with WT (−), KO (−) or KO (+).

FIG. 15. Epithelial-mesenchymal transition and TGF-β1 upregulation under a mechanical environment. (a-d) Dual immunofluorescence of E-cadherin (green) and α-smooth muscle actin (red) in renal tubular epithelial cells derived from wild-type (WT) (a and b) and Smad3-null (KO) mice (c and d) stretched for 24 h in the absence (a and c) or presence (b and d) of a neutralizing anti-TGF-β1 antibody (20 μg/ml). Scale bar, 20 μm. (e) Northern blot of Snail mRNA in the epithelial cells either stretched for 24 h or non-stretched in the absence or presence of a neutralizing anti-TGF-β1 antibody. Similar results were obtained from additional two experiments. (f) Northern blot of TGF-β1 mRNA in primary culture of the epithelial cells either stretched for 24 h or non-stretched in the absence or presence of a neutralizing anti-TGF-β1 antibody. Results are means±standard deviation of 5 samples. *P<0.01 as compared with other experimental groups. (g) Total TGF-β1 concentration in culture medium of the epithelial cells either stretched or non-stretched. Results are means±standard deviation of 5 samples. *P<0.05 as compared with non-stretched counterparts.

FIG. 16. Role of exogenous monocytes in epithelial-mesenchymal transition of renal tubular epithelial cells. (a-d) Dual immunofluorescence of E-cadherin (green) and α-smooth muscle actin (α-SMA) (red) in co-culture of renal tubular epithelial cells and bone-marrow monocytes for 48 h. (a) Co-culture of wild-type (WT) epithelial cells and WT monocytes. (b) WT epithelial cells and Smad3-null (KO) monocytes. (c) KO epithelial cells and WT monocytes. (d) KO epithelial cells and KO monocytes. (e-l) Transplantation of monocytes into the subcapsular space of the kidney immediately before UUO for 3 days. Dotted lines indicate the border between the subcapsular space (left) and the renal cortex (right). (e-h) Immunofluorescence of F4/80 antigen (green). (i-l) Dual immunofluorescence of E-cadherin (green) and α-SMA (red). (e and i) Transplantation of WT monocytes to WT kidneys. (f and j) KO monocytes to WT kidneys. (g and k) WT monocytes to KO kidneys. (h and l) KO monocytes to KO kidneys. DAPI (blue) was used for nuclear staining. Scale bar, 20 μm. Similar results were obtained from four additional experiments.

FIG. 17. Smad3 is required for transition of retinal pigment epithelial (RPE) cells to a fibroblastic-like morphology following retinal detachment. Hematoxylin and eosin-stained paraffin sections of eyes at Week 1 (a-d), 2 (e, f) and 8 (g, h) post-retinal detachment. Panels c-f show high power magnification of the posterior part (boxed areas) of the eye. Panels in the left column (a, c, e, g) or those in the right column (b, d, f, h) show histology of Smad3^{+/+} (WT) or Smad3^{ex8/ex8} (KO) mouse eyes, respectively. At Weeks 1, 2 and 8, RPE cells in the posterior pole region of WT eyes formed a focal multilayered structure (c, e, g), whereas RPE cells retained their monolayer pattern in KO retinas (d, f, h). Frames c and d are high magnification pictures of the boxed area in frames a and b, respectively. Fibroblast-like RPE cells appeared to be less pigmented at Week 8 as compared with those at Weeks 1 and 2 in WT mice (c, e, g). Bar, 150 μm (a, b), 20 μm (c-h).

FIG. 18. Snail is an early Smad3-dependent marker of EMT in WT retinal pigment epithelial (RPE) cells following retinal detachment. Expression of snail mRNA in RPE cells at day 2 (a, b), Week 1 (c, d), and Week 8 (e, f) post-retinal detachment in either Smad3^{+/+} (WT) (a, c, e) or Smad3^{ex8/ex8} (KO) (b, d, f) eyes. Expression of snail mRNA was not detectable 2 days (a, b), but could be seen in the multilayered plaque formed under the detached retina by WT RPE cells at week 1 (c) or week 8 (e) post-retinal detachment. KO RPE cells never expressed snail mRNA throughout the intervals examined up to Week 8 (d, f). No signal was seen with the sense riboprobe (Insert in e). Arrows indicate cell nuclei positive (c, e) or negative (d, f) for snail mRNA, respectively. In situ hybridization with a digoxigenin-alkaline phosphatase reaction. Bar, 20 μm.

FIG. 19. Smad3 is required for expression of αSMA protein in retinal pigment epithelial (RPE) cells following retinal detachment. Left or right columns represent WT or KO eyes, respectively. Uninjured RPE cells (a, b) were negative for αSMA protein in both Smad3^{+/+} (WT) and Smad3^{ex8/ex8} (KO) mice. Two weeks post-retinal detachment, elongated, multilayered, mesenchymal-like pigmented cells were labeled with anti-αSMA antibody (c) in WT eyes, whereas monolayer RPE cells of KO eyes (d) or WT eyes, were not labeled. At week 8 (e, f), prominent focal fibrous tissue including pigmented cells of a fibroblastic appearance in WT eyes were markedly positive for αSMA (e), whereas RPE cells in KO mice neither form cell multilayers in the posterior region nor express αSMA in pigment epithelial layer (f). Immunofluorescence staining with DAPI nuclear staining, bar, 50 μm.

FIG. 20. Smad 3 is required for expression of extracellular matrix components laminin, lumican and collagen type VI in subretinal fibrotic tissue formed following retinal detachment. Immunofluorescence staining for laminin (LN, a, d, g, j), lumican (Lum, b, e, h, k) and collagen type VI (Col VI, c, f, i, l) in RPE cells in the posterior region following retinal detachment. Panels a-c and j-l represent Smad3^{ex8/ex8} (KO) eyes and those of d-i Smad3^{+/+} (WT) mice. Laminin, collagen VI and lumican were not detected in RPE cells of an uninjured eye of WT or KO mouse (a-c), although weak staining for laminin was detected in Bruch's membrane and choroidal vessels (a), and lumican (b), and collagen VI (c), were observed in scleral matrix. At Week 1 post-retinal detachment, lumican and collagen VI were expressed in αSMA-positive multilayered fibroblast-like RPE cells in WT eyes (d-f), but not in KO RPE cells. Laminin immunolocalization is restricted to Bruch's membrane in a WT eye (d). At Week 8, laminin, lumican and collagen VI each stained positively in the fibrous tissue formed under the detached retina in WT eyes (g-i), whereas they were not expressed in RPE cells in KO mice at these same timepoints (j-l). Immunofluorescence staining with DAPI nuclear staining, bar, 100 μm.

FIG. 21. Epithelial-mesenchymal transition and Smad signaling of cultured retinal pigment epithelial cells (RPE cells). Primary porcine RPE cells cultured on fibronectin do not express αSMA (a) but, undergo EMT, as reveled by αSMA expression, following exposure to TGF-β2 for 48 hr (b). ARPE-19 cells express αSMA in response to TGF-β addition at 72 hr (c). In this cell type, Smads2/3 are phosphorylated within 30 min after TGF-β2 addition (d) and nuclear translocation of Smad3 is also observed within 0.5 hr with maximal levels 1 hr after TGF-β2 addition (e). Indirect immunostaining by diaminobenzidine color reaction methylgreen counterstaining (a, b) and immunofluorescence staining with DAPI nuclear staining (c, e), bar, 100 μm (a, b), 50 μm (c, e).

FIG. 22. Cell migration is associated with Smad3 activation and exogenous TGF-β2 accelerates migration of ARPE-19 cells. a. Following wounding of a monolayer of ARPE-19 cells, nuclear translocation of Smad3 was observed in cells near the wounded edge beginning 1 hr after injury and increasing to maximal level 3 to 7 hrs post-wounding. Arrowheads or arrows indicate weak or obvious staining for nuclear Smad3 in ARPE-19 cells, respectively. No nuclear Smad3 is detected at 24 hr post-wounding. The results indicate that ARPE-19 cells are activated by endogenous TGF-β post-wounding via autocrine or paracrine fashion. b. Migration of ARPE-19 cells is accelerated by adding TGF-β2 (1.0 ng/ml) to the culture medium. The cleared defect in wounded ARPE-19 monolayers is filled within 12 hr in cultures treated with TGF-β2, compared to 24 hr in untreated control cultures. Immunofluorescence staining with DAPI nuclear staining (a) and hematoxylin and eosin staining (b), bar, 50 μm (a), 200 μm (b).

FIG. 23. Induction of αSMA by TGF-β2 in organ-cultured RPE cells post-injury requires Smad3. Following 48 hrs in culture with TGF-β2, injured RPE cells on the Bruch's membrane (dotted line) of a Smad3^{+/+} (WT), but not in Smad3^{ex8/ex8} (KO) posterior eye segments stain positively for αSMA (arrows). Immunofluorescence staining with DAPI nuclear staining, bar, 10 μm.

FIG. 24. Increment of cell proliferation in retinal pigment epithelial (RPE) cells and PDGF-BB expression in Smad3^{+/+} (WT), mice, but not seen in Smad3^{ex8/ex8} (KO) mice, post-retinal detachment. PCNA-positive RPEs were observed in cell multilayers formed in WT mice at Week 1 (aA) and 2, but not at week 4 and 8. No PCNA-positive cells were detected in RPE cells immediately after retinal detachment induction in a WT mouse or in RPE cells of KO mouse at any timepoint (aB, at Week 1). Frame b shows the number of PCNA-positive RPE cells in posterior part of the eye at Week 1 and 2 following retinal detachment. More PCNA-labeled cells are detected in WT eyes as compared with KO eyes. Newly formed PVR tissue in WT mice containing fibroblast-like RPE cells were labeled with anti-PDGF-BB antibody at all times examined after Week 1 post-retinal detachment (cA), while RPE cells in KO mice neither formed a cell multilayer nor expressed PDGF-BB (cB). White dotted lines, Bruch's membrane. Immunofluorescence staining with DAPI nuclear staining (a, c), bar, 10 μm.

FIG. 25. TGF-β2 induces expression of PDGF in ARPE-19 cells that modulates its effects on cell proliferation. a. Western blot of PDGF-B in ARPE-19 cells treated with 1.0 ng/ml of TGF-β2 for 0-96 hrs. PDGF-B chain is detected at 24 hr culture increases up to 96 hrs after addition of TGF-β2. b. Total amount of PDGF-BB and PDGF-AB in culture medium detected by using an enzyme-immunosorbent assay. TGF-β2 stimulates production of PDGF-BB and -AB by the cells. c. We then examined effects of TGF-β2, PDGF-BB and TGF-β2 plus anti-PDGF-B antibody on cell proliferation of ARPE-19 cells. PDGF-BB (5 ng/ml) enhanced and TGF-β2 (1 ng/ml) inhibited the growth of the cells. Addition of a PDGF-B neutralizing antibody (20 μg/ml) to TGF-β2 culture resulted in further suppression of cell proliferation at later timepoints of 120 hr culture, indicating that the accumulation of endogenous PDGF-BB counteracts the growth inhibitory effects of exogenous TGF-β2.

FIG. 26. TGF-β2 enhances expression of TGF-β1 and type I collagen in ARPE-19 cells. a. An enzyme-immunoassay shows an increment of amount of TGF-β1 in medium of ARPE-19 cells treated with exogenous TGF-β2. b. Immunofluorescent staining shows increased cytoplasmic fluorescence and pericellular deposition of type I collagen in TGF-β2-treated cultures as compared with the control. c. Both TGF-β1 and, to a somewhat lesser extent, TGF-β2 increase the amount of type I collagen in both culture medium and cell lysate as determined by using an enzyme-linked immunosorbent assay. Immunofluorescence staining with DAPI nuclear staining (b), bar, 20 μm.

FIG. 27. Model of development of proliferative vitreoretinopathy following retinal detachment in the mouse eye. Following retinal detachment in a Smad3^{+/+} (WT) eye, retinal pigment epithelial (RPE) cells undergo epithelial-mesenchymal transition (EMT) and form multilayers of αSMA-positive mesenchymal-like cells which express extracellular matrix under the detached retina. Similar changes are not seen in RPE cells in Smad3^{ex8/ex8} (KO) eyes, demonstrating a dependence of these processes on the Smad3 pathway. Cell proliferation was seen in peripheral areas of the subretinal space in both WT and KO eyes, but, like cells in the posterior zone, these cells do not express EMT in KO eyes.

### DETAILED DESCRIPTION OF THE PREFERRED EMBODIMENT

Regarding the TGF-/Smad pathway, upon ligand-induced heteromeric complex formation and activation by type II kinase receptors of type I receptor kinases, R-Smads are phosphorylated. Several proteins with anchoring, scaffolding, and/or chaperone activity have been identified. The activated R-Smads form heteromeric complexes with Co-Smads and accumulate in the nucleus. Together with co-activators, co-repressors, and transcription factors, these Smad complexes participate in transcriptional regulation of target genes. Ligands include activins, AMH, BMPs, and TGF-βs. Type II receptors include ActR-II, ActR-IIB, AMHR-II, BMPR-II, and TβR-II. Type I receptors include ALK1˜7. R-(receptor-regulated-) Smads include Smad 1, 2, 3, 5, and 8. I- (inhibitory-) Smads include Smad 6 and 7. Co-Smads include Smad 4α and β. Scaffolding proteins include Axil, Axin, Caveolin-1, Dab-2, Hrs/Hgs, SARA, SNIX, Strap, TLP, and TRAP-1. Cytoskeletal components include filamin-1 and tubulin. Nuclear transporters include CRM1, Importinβ, and Ran GTPase. Transcriptional regulators include AR, ATF-2, BF-1, E1A, ER, Evi-1, FAST/FosH1, c-Fos, Gli3, GR, c-Jun, JunB, JunD, HNF4, LEF/TCF, MEF2, Menin, Milk, Mixer, Miz-1, MyoD, OAZ, p52, PEBP2/CBFA/AML, pX, SNIP1, Sp1, Sp3, Tax1, TFE3, and VDR. Transcriptional co-activators include MSG1, p300/CBP, and P/CAF. Transcriptional repressors include Hoxa-9 and Hoxc-8. Transcriptional co-repressors include HDACs, Ski, SnoN, and TGIF.

List of abbreviations: ActR-II, activin type II receptor; ActR-IIB, activin type IIB receptor; ALK, activin-receptor-like kinase; AMH, anti-Müllerian hormone; AMHR-II, AMH type II receptor; AR, androgen receptor; ATF-2, activating transcription factor-2; BF-1, brain factor-1; BMPs, bone morphogenetic proteins; BMPR-II, BMP type II receptor; CBP, CREB-binding protein; CREB, cAMP-responsive element-binding protein; CRM1, chromosome region maintenance 1; Dab-2, disabled-2; E1A, early region 1A; Evi-1, ectopic viral integration site-1; ER, estrogen receptor; FAST/FoXH1, forkhead activin signal transducer; Gli3, glioblastoma gene product 3; GR, glucocorticoid receptor; HDACs, histone deacetylases; HNF4, hepatocyte nuclear factor 4; Hoxa-9, homeobox gene a-9; Hoxc-8, homeobox gene c-8; Hrs/Hgs, hepatic growth factor-regulated tyrosine kinase substrate; LEF1/TCF, lymphoid enhancer factor/T-cell factor; MEF2, myocyte enhancer-binding factor 2; Menin, multiple endocrine neoplasia-type 1 tumor suppressor protein; Miz-1, Myc-interacting zinc-finger protein 1; MSG1, melanocyte-specific gene 1; OAZ, Olf-1/EBF associated zinc-finger; PEBP2/CBFA/AML, polyomavirus-enhancer-binding protein/core-binding factor A/acute myeloid leukemia; P/CAF, p300/CBP-associated factor; SARA, Smad anchor for receptor activation; Ski, Sloan-Kettering avian retrovirus; SNIP1, Smad nuclear interacting protein 1; SnoN, ski-related novel gene; SNX, sorting nexin; Sp1, specificity protein 1; Sp3, specificity protein 3; STRAP, serine-threonine kinase receptor-associated protein; TR-II, TGF-type II receptor; TFE3, transcription factor mu E3; TGF-s, transforming growth factor-s; TGIF, 5TG3-interacting factor; TLP, TRAP-1-like protein; TRAP-1, TGF-receptor-associated protein-1; VDR, vitamin D receptor.

Definitions

The term “isolated” requires that a material be removed from its original environment (e.g., the natural environment if it is naturally occurring). For example, a naturally occurring polynucleotide or polypeptide present in a living cell is not isolated, but the same polynucleotide or polypeptide, separated from some or all of the coexisting materials in the natural system, is isolated.

The term “purified” does not require absolute purity; rather it is intended as a relative definition, with reference to the purity of the material in its natural state. Purification of natural material to at least one order of magnitude, preferably two or three magnitudes, and more preferably four or five orders of magnitude is expressly contemplated.

The term “enriched” means that the concentration of the material is at least about 2, 5, 10, 100, or 1000 times its natural concentration (for example), advantageously 0.01% by weight. Enriched preparations of about 0.5%, 1%, 5%, 10%, and 20% by weight are also contemplated.

The Smad3 Gene

To date, nine vertebrate Smads have been identified, and these have been divided into subgroups based on their functional role in various pathways. Smad1, 5, and Smad8, all mediate signal transduction from BMPs, while Smad2 and Smad3 mediate signal transduction from TGF-βs and activins. Collectively, these Smads are known as the pathway-restricted Smads and can form homo or heterodimers. Smad4 has been shown to be a shared hetero-oligomerization partner to the pathway-restricted Smads and is known as the common mediator. The last two members of the family, Smad6 and 7, act to inhibit the Smad signaling cascades often by forming unproductive dimers with other Smads and are therefore classified as antagonistic Smads (Heldin et al., Nature, 1997, 390, 465-471; Kretzschmar and Massague, Curr. Opin. Genet. Dev., 1998, 8, 103-111).

The published amino acid sequence of human Smad3 is provided as GenBank accession number NP_{—}005893. The published cDNA sequence of human Smad3 is available as GenBank accession number U68019. The genomic sequence is also known.

The Smad3 nucleotide sequences of the invention include: (a) the cDNA sequence given in GenBank accession number U68019; (b) the nucleotide sequence that encodes the amino acid sequence given in GenBank accession number NP_{—}005893; (c) any nucleotide sequence that hybridizes to the complement of the cDNA sequence given in GenBank accession number U68019 under highly stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (e.g., see Ausubel F. M. et al., eds., 1989, Current Protocols in Molecular Biology, Vol. 1, Green Publishing Associates, Inc., and John Wiley & sons, Inc., New York, at p. 2.10.3) and encodes a functionally equivalent gene product; and (d) any nucleotide sequence that hybridizes to the complement of the cDNA sequence given in GenBank accession number U68019 under less stringent conditions, such as moderately stringent conditions, e.g., washing in 0.2×SSC/0.1% SDS at 42° C. (Ausubel et al., 1989, supra), yet which still encodes a functionally equivalent gene product. Functional equivalents of Smad3 include naturally occurring Smad3 present in other species, and mutant Smad3s whether naturally occurring or engineered. Aspects of the invention also include degenerate variants of sequences (a) through (d).

Embodiments of the invention also include nucleic acid molecules, preferably DNA molecules, that hybridize to, and are therefore the complements of, the nucleotide sequences (a) through (d), in the preceding paragraph. Such hybridization conditions may be highly stringent or less highly stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60 C. (for 23-base oligos). These nucleic acid molecules may encode or act as Smad3 antisense molecules, useful, for example, in Smad3 gene regulation (for and/or as antisense primers in amplification reactions of Smad3 gene nucleic acid sequences). Further, such sequences can be used as part of ribozyme and/or interfering RNA sequences, also useful for Smad3 gene regulation.

In addition to the Smad3 nucleotide sequences described above, full length Smad3 cDNA or gene sequences present in the same species and/or homologs of the Smad3 gene present in other species can be identified and readily isolated, without undue experimentation, by molecular biological techniques well known in the art. The identification of homologs of Smad3 in related species can be useful for developing animal model systems more closely related to humans for purposes of drug discovery. For example, expression libraries of cDNAs synthesized from mRNA derived from the organism of interest can be screened using labeled TGF-β or activin receptors (or Smads involved in forming dimers with Smad3) derived from that species. Alternatively, such cDNA libraries, or genomic DNA libraries derived from the organism of interest can be screened by hybridization using the nucleotides described herein as hybridization or amplification probes. Furthermore, genes at other genetic loci within the genome that encode proteins, which have extensive homology to one or more domains of the Smad3 gene product, can also be identified via similar techniques. In the case of cDNA libraries, such screening techniques can identify clones derived from alternatively spliced transcripts in the same or different species.

Screening can be by filter hybridization, using duplicate filters. The labeled probe can contain at least 15-30 base pairs of the Smad3 cDNA sequence. The hybridization washing conditions used should be of a lower stringency when the cDNA library is derived from an organism different from the type of organism from which the labeled sequence was derived. With respect to the cloning of a human Smad3 homolog, using murine Smad3 probes, for example, hybridization can, for example, be performed at 65° C. overnight in Church's buffer (7% SDS, 250 mM NaHPO₄, 2 μM EDTA, 1% BSA). Washes can be done with 2×SSC, 0.1% SDS at 65° C. and then at 0.1×SSC, 0.1% SDS at 65° C.

Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

Alternatively, the labeled Smad3 nucleotide probe can be used to screen a genomic library derived from the organism of interest, again, using appropriately stringent conditions. The identification and characterization of human genomic clones is helpful for designing clinical protocols in human patients. For example, sequences derived from regions adjacent to the intron/exon boundaries of the human gene can be used to design primers for use in amplification assays to detect mutations within the exons, introns, splice sites (e.g. splice acceptor and/or donor sites), etc.

Further, a Smad3 gene homolog may be isolated from nucleic acid of the organism of interest by performing PCR using two degenerate oligonucleotide primer pools designed on the basis of amino acid sequences within the Smad3 gene product disclosed herein. The template for the reaction may be cDNA obtained by reverse transcription of mRNA prepared from, for example, human or non-human cell lines or tissue known or suspected to express a Smad3 gene allele.

The PCR product may be subcloned and sequenced to ensure that the amplified sequences represent the sequences of a Smad3 gene. The PCR fragment may then be used to isolate a full length cDNA clone by a variety of methods. For example, the amplified fragment may be labeled and used to screen a cDNA library, such as a bacteriophage cDNA library. Alternatively, the labeled fragment can be used to isolate genomic clones via the screening of a genomic library.

PCR technology may also be utilized to isolate full length cDNA sequences. For example, RNA can be isolated, following standard procedures, from an appropriate cellular or tissue source (i.e., one known, or suspected, to express the Smad3 gene). A reverse transcription reaction may be performed on the RNA using an oligonucleotide primer specific for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Accordingly, cDNA sequences upstream of the amplified fragment can be isolated. For a review of cloning strategies that may be used, see e.g., Sambrook et al., 1989, supra.

The Smad3 gene sequences can additionally be used to isolate mutant Smad3 gene alleles. Such mutant alleles can be isolated from individuals either known or proposed to have a genotype that contributes to Smad3 mediated disorders. Mutant alleles and mutant allele products can then be utilized in the therapeutic systems described below. Additionally, such Smad3 gene sequences can be used to detect Smad3 gene regulatory (e.g., promoter or promotor/enhancer) defects.

A cDNA of a mutant Smad3 gene can be isolated, for example, by using PCR. In this case, the first cDNA strand can be synthesized by hybridizing an oligo-dT oligonucleotide to mRNA isolated from tissue known or suspected to be expressed in an individual putatively carrying the mutant Smad3 allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant Smad3 allele to that of the normal Smad3 allele, the mutation(s) responsible for the loss or alteration of function of the mutant Smad3 gene product can be ascertained.

Alternatively, a genomic library can be constructed using DNA obtained from an individual suspected of or known to carry the mutant Smad3 allele, or a cDNA library can be constructed using RNA from a tissue known, or suspected, to express the mutant Smad3 allele. The normal Smad3 gene or any suitable fragment thereof may then be labeled and used as a probe to identify the corresponding mutant Smad3 allele in such libraries. Clones containing the mutant Smad3 gene sequences can then be purified and subjected to sequence analysis according to methods well known to those of skill in the art.

Additionally, an expression library can be constructed utilizing cDNA synthesized from, for example, RNA isolated from a tissue known, or suspected, to express a mutant Smad3 allele in an individual suspected of or known to carry such a mutant allele. In this manner, gene products made by the putatively mutant tissue can be expressed and screened using standard antibody screening techniques in conjunction with antibodies raised against the normal Smad3 gene product, as described, below, in the appropriate sections. (For screening techniques, see, for example, Harlow, E. and Lane, eds., 1988, “Antibodies: A Laboratory Manual”, Cold Spring Harbor Press, Cold Spring Harbor.) Additionally, screening can be accomplished by screening with labeled Smad3 fusion proteins. In cases where a Smad3 mutation results in an expressed gene product with altered function (e.g., as a result of a missense or a frameshift mutation), a polyclonal set of antibodies to Smad3 are likely to cross-react with the mutant Smad3 gene product. Library clones detected via their reaction with such labeled antibodies can be purified and subjected to sequence analysis according to methods well known to those of skill in the art.

Aspects of the invention also concern nucleotide sequences that encode mutant Smad3s, peptide fragments of Smad3, truncated Smad3s, and Smad3 fusion proteins. These include, but are not limited to, nucleotide sequences encoding mutant Smad3s described in subsequent sections or peptides corresponding to a domain of Smad3 or portions of these domains; truncated Smad3s in which one or two of the domains is deleted, or a truncated, nonfunctional Smad3 lacking all or a portion of a domain. Nucleotides encoding fusion proteins may include, but are not limited to, full length Smad3, truncated Smad3 or peptide fragments of Smad3 fused to an unrelated protein or peptide, such as for example, a transmembrane sequence, which anchors the Smad3 to the cell membrane; an Ig Fc domain, which increases the stability and half life of the resulting fusion protein in the bloodstream; or an enzyme, fluorescent protein, luminescent protein, which can be used as a marker.

Embodiments of the invention also concern (a) DNA vectors that contain any of the foregoing Smad3 coding sequences and/or their complements (i.e., antisense); (b) DNA expression vectors that contain any of the foregoing Smad3 coding sequences operatively associated with a regulatory element that directs the expression of the coding sequences; and (c) genetically engineered host cells that contain any of the foregoing Smad3 coding sequences operatively associated with a regulatory element that directs the expression of the coding sequences in the host cell. As used herein, regulatory elements include, but are not limited to, inducible and non-inducible promoters, enhancers, operators and other elements known to those skilled in the art that drive and regulate expression. Such regulatory elements include, but are not limited to, the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeastα-mating factors.

Particular polynucleotides are DNA sequences having three sequential nucleotides, four sequential nucleotides, five sequential nucleotides, six sequential nucleotides, seven sequential nucleotides, eight sequential nucleotides, nine sequential nucleotides, ten sequential nucleotides, eleven sequential nucleotides, twelve sequential nucleotides, thirteen sequential nucleotides, fourteen sequential nucleotides, fifteen sequential nucleotides, sixteen sequential nucleotides, seventeen sequential nucleotides, eighteen sequential nucleotides, nineteen sequential nucleotides, twenty sequential nucleotides, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty,-six, forty-seven, forty-eight, forty-nine, fifty, fifty-one, fifty-two, fifty-three, fifty-four, fifty-five, fifty-six, fifty-seven, fifty-eight, fifty-nine, sixty, sixty-one, sixty-two, sixty-three, sixty-four, sixty-five, sixty-six, sixty-seven, sixty-eight, sixty-nine, seventy, seventy-one, seventy-two, seventy-three, seventy-four, seventy-five, seventy-six, seventy-seven, seventy-eight, seventy-nine, eighty, ninety, one-hundred, two-hundred, or three-hundred or more sequential nucleotides.

Smad3 Proteins and Polypeptides

Smad3 protein, polypeptides and peptide fragments, mutated, truncated or deleted forms of Smad3 and/or Smad3 fusion proteins can be prepared for a variety of uses, including but not limited to, the generation of antibodies, as reagents for research purposes, or the identification of other cellular gene products involved in the regulation of Smad3 mediated processes, as reagents in assays for screening for compounds that can be used in the treatment of Smad3 mediated disorders, and as pharmaceutical reagents useful in the treatment of disorders mediated by Smad3.

The Smad3 amino acid sequences of the invention include the amino acid sequence given in GenBank accession number NP_{—}005893, or the amino acid sequence encoded by the cDNA or encoded by the gene. Further, Smad3 of other species are encompassed by the invention. In fact, any Smad3 encoded by the Smad3 nucleotide sequences described in the sections above are within the scope of the invention.

Aspects of the invention also encompass proteins that are functionally equivalent to Smad3 encoded by the nucleotide sequences described in the above sections, as judged by any of a number of criteria, including but not limited to, the ability to bind TGF-β or activin receptors or Smads involved in forming dimers with Smad3, the binding affinity for these ligands, the resulting biological effect of Smad3 binding, e.g., signal transduction, a change in cellular metabolism or change in phenotype when the Smad3 equivalent is present in an appropriate cell type, or the regulation of Smad3 mediated processes. Such functionally equivalent Smad3 proteins include, but are not limited to, additions or substitutions of amino acid residues within the amino acid sequence encoded by the Smad3 nucleotide sequences described in the sections above, but which result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. While random mutations can be made to Smad3 DNA (using random mutagenesis techniques well known to those skilled in the art) and the resulting mutant Smad3s tested for activity, site-directed mutations of the Smad3 coding sequence can be engineered (using site-directed mutagenesis techniques well known to those skilled in the art) to generate mutant Smad3s with altered function, e.g., different binding affinity for TGF-β or activin receptors or Smads involved in forming dimers with Smad3, and/or different signalling capacity.

For example, identical amino acid residues of a mouse form of Smad3 and the human Smad3 homolog can be aligned so that regions of identity are maintained, whereas the variable residues are altered, e.g., by deletion or insertion of an amino acid residue(s) or by substitution of one or more different amino acid residues. Conservative alterations at the variable positions can be engineered in order to produce a mutant Smad3 that retains function; e.g., ligand binding affinity or signal transduction capability or both. Non-conservative changes can be engineered at these variable positions to alter function, e.g., ligand binding affinity or signal transduction capability, or both. Alternatively, where alteration of function is desired, deletion or non-conservative alterations of the conserved regions (i.e., identical amino acids) can be engineered. For example, deletion or non-conservative alterations (substitutions or insertions) of a domain can be engineered to produce a mutant Smad3 that binds a ligand but is signalling-incompetent. Non-conservative alterations to residues of identical amino acids can be engineered to produce mutant Smad3s with altered binding affinity for ligands. The same mutation strategy can also be used to design mutant Smad3s based on the alignment of other non-human Smad3s and the human Smad3 homolog by aligning identical amino acid residues.

Other mutations to the Smad3 coding sequence can be made to generate Smad3s that are better suited for expression, scale up, etc. in the host cells chosen. For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts, which are known to hyperglycosylate N-linked sites.

Peptides corresponding to one or more domains of Smad3, as well as fusion proteins in which the full length Smad3, a Smad3 peptide or truncated Smad3 is fused to an unrelated protein, are also within the scope of the invention and can be designed on the basis of the Smad3 amino acid sequences given in GenBank accession number NP_{—}005893. Such fusion proteins include but are not limited to IgFc fusions, which stabilize the Smad3 protein or peptide and prolong half-life in vivo; or fusions to any amino acid sequence that allows the fusion protein to be anchored to the cell membrane; or fusions to an enzyme, fluorescent protein, or luminescent protein, which provide a marker function.

While the Smad3 polypeptides and peptides can be chemically synthesized (e.g., see Creighton, 1983, Proteins: Structures and Molecular Principles, W. H. Freeman & Co., N.Y.), large polypeptides derived from Smad3 and the full length Smad3 itself may advantageously be produced by recombinant DNA technology using techniques well known in the art for expressing nucleic acid containing Smad3 gene sequences and/or coding sequences. Such methods can be used to construct expression vectors containing the Smad3 nucleotide sequences and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding Smad3 nucleotide sequences may be chemically synthesized using, for example, synthesizers. See, for example, the techniques described in “Oligonucleotide Synthesis”, 1984, Gait, M. J. ed., IRL Press, Oxford.

A variety of host-expression vector systems can be utilized to express the Smad3 nucleotide sequences described herein. Where the Smad3 peptide or polypeptide is soluble, the peptide or polypeptide can be recovered from the culture, e.g., from the host cell in cases where the Smad3 peptide or polypeptide is not secreted, and from the culture media in cases where the Smad3 peptide or polypeptide is secreted by the cells. However, the expression systems also encompass engineered host cells that express the Smad3 or functional equivalents in situ, e.g., anchored in the cell membrane. Purification or enrichment of the Smad3 from such expression systems can be accomplished using appropriate detergents and lipid micelles and methods well known to those skilled in the art. However, such engineered host cells themselves may be used in appropriate situations.

The expression systems that may be used with some embodiments include, but are not limited to, microorganisms such as bacteria (e.g., E. coli, B. subtilis) transformed with recombinant bacteriophage DNA, plasmid DNA or cosmid DNA expression vectors containing Smad3 nucleotide sequences; yeast (e.g., Saccharomyces, Pichia) transformed with recombinant yeast expression vectors containing the Smad3 nucleotide sequences; insect cell systems infected with recombinant virus expression vectors (e.g., baculovirus) containing the Smad3 sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing Smad3 nucleotide sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the Smad3 gene product being expressed. For example, when a large quantity of such a protein is to be produced, for the generation of pharmaceutical compositions of Smad3 protein or for raising antibodies to the Smad3 protein, for example, vectors, which direct the expression of high levels of fusion protein products that are readily purified may be desirable. Such vectors include, but are not limited, to the E. coli expression vector pUR278 (Ruther et al., 1983, EMBO J. 2:1791), in which the Smad3 coding sequence may be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign genes. The virus grows in Spodoptera frugiperda cells. The Smad3 gene coding sequence may be cloned individually into non-essential regions (for example the polyhedrin gene) of the virus and placed under control of an AcNPV promoter (for example the polyhedrin promoter). Successful insertion of Smad3 gene coding sequence will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted gene is expressed. (E.g., see Smith et al. 1983 J Virol 46:584; Smith, U.S. Pat. No. 4,215,051.)

In mammalian host cells, a number of viral-based expression systems may be utilized. In cases where an adenovirus is used as an expression vector, the Smad3 nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the Smad3 gene product in infected hosts. (E.g., See Logan & Shenk 1984 PNAS USA 81:3655-3659). Specific initiation signals may also be required for efficient translation of inserted Smad3 nucleotide sequences. These signals include the ATG initiation codon and adjacent sequences. In cases where an entire Smad3 gene or cDNA, including its own initiation codon and adjacent sequences, is inserted into the appropriate expression vector, no additional translational control signals may be needed. However, in cases where only a portion of the Smad3 coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al. 1987 Methods in Enzymol 153:516-544).

In addition, a host cell strain that modulates the expression of the inserted sequences, or modifies and processes the gene product in the specific fashion desired may be chosen. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells, which possess the cellular machinery for proper processing of the primary transcript, glycosylation, and phosphorylation of the gene product, may be used. Such mammalian host cells include but are not limited to CHO, VERO, BHK, HeLa, COS, MDCK, 293, 3T3, and WI38.

For long-term, high-yield production of recombinant proteins, stable expression is preferred. For example, cell lines, which stably express the Smad3 sequences described above, may be engineered. Rather than using expression vectors, which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines, which express the Smad3 gene product. Such engineered cell lines may be particularly useful in screening and evaluation of compounds that affect the endogenous activity of the Smad3 gene product.

A number of selection systems may be used, including but not limited to the herpes simplex virus thymidine kinase (Wigler, et al. 1977 Cell 11:223), hypoxanthine-guanine phosphoribosyltransferase (Szybalska & Szybalski 1962 PNAS USA 48:2026), and adenine phosphoribosyltransferase (Lowy, et al. 1980 Cell 22:817) genes can be employed in tk-, hgprt- or aprt-cells, respectively. Also, antimetabolite resistance can be used as the basis of selection for the following genes: dhfr, which confers resistance to methotrexate (Wigler, et al. 1980 PNAS USA 77:3567; O'Hare, et al. 1981 PNAS USA 78:1527); gpt, which confers resistance to mycophenolic acid (Mulligan & Berg 1981 PNAS USA 78:2072); neo, which confers resistance to the aminoglycoside G-418 (Colberre-Garapin, et al. 1981 J Mol Biol 150:1); and hygro, which confers resistance to hygromycin (Santerre, et al. 1984 Gene 30:147).

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al. 1991 PNAS USA 88:8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

The Smad3 gene products can also be expressed in transgenic animals. Animals of any species, including, but not limited to, mice, rats, rabbits, guinea pigs, pigs, micro-pigs, goats, and non-human primates, e.g., baboons, monkeys, and chimpanzees may be used to generate Smad3 transgenic animals.

Particular polypeptides are amino acid sequences having three sequential residues, four sequential residues, five sequential residues, six sequential residues, seven sequential residues, eight sequential residues, nine sequential residues, ten sequential residues, eleven sequential residues, twelve sequential residues, thirteen sequential residues, fourteen sequential residues, fifteen sequential residues, sixteen sequential residues, seventeen sequential residues, eighteen sequential residues, nineteen sequential residues, twenty sequential residues, twenty-one, twenty-two, twenty-three, twenty-four, twenty-five, twenty-six, twenty-seven, thirty, forty, fifty, sixty, seveny, eighty, ninety, or more sequential residues.

Screening Assays for Compounds that Inhibit Smad3 Expression or Activity

The following assays are designed to identify compounds that inhibit Smad3, compounds that interfere with the interaction of Smad3 with intracellular proteins, and compounds that interfere with the interaction of Smad3 with transmembrane proteins, e.g., TGF-β and activin receptors, and compounds that inhibit the activity of the Smad3 gene or modulate the level of Smad3. Assays may additionally be utilized that identify compounds that bind to Smad3 gene regulatory sequences (e.g., promoter sequences) and that may inhibit Smad3 gene expression. Assays may additionally be utilized to identify compounds that interfere with the interaction of Smad3 with promoters of target genes.

The compounds that may be screened in accordance with these embodiments include, but are not limited to: peptides and analogues thereof, carbohydrates, lipids, nucleic acid sequences such as aptamers, antibodies and fragments thereof, and small organic compounds (e.g., peptidomimetics) and inorganic compounds that bind to Smad3, or to intracellular proteins that interact with Smad 3, or to transmembrane proteins that interact with Smad3 and inhibit the activity triggered by Smad3 or mimic the inhibitors of Smad3; as well as peptides or analogues thereof, antibodies or fragments thereof, and other organic compounds that mimic the ligands of Smad3 (or a portion thereof) and bind to and “neutralize” Smad3.

Such compounds may include, but are not limited to, peptides such as, for example, soluble peptides, including but not limited to members of random peptide libraries; (see, e.g., Lam, K. S. et al. 1991 Nature 354:82-84; Houghten, R. et al. 1991 Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang, Z. et al. 1993 Cell 72:767-778), antibodies (including, but not limited to, polyclonal, monoclonal, humanized, anti-idiotypic, chimeric or single chain antibodies, and FAb, F(ab′)2 and FAb expression library fragments, and epitope-binding fragments thereof), and small organic or inorganic molecules.

Analogues of peptides contemplated herein include, but are not limited to, modifications to side chains, incorporation of unnatural amino acids and/or their derivatives during peptide synthesis and the use of crosslinkers, and other methods which impose conformational constraints on the peptides or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6, trinitrobenzene sulphonic acid (TNBS); acylation of amino groups with succinic anhydride and tetrahydrophthalic anhydride; and pyridoxylation of lysine with pyridoxal-5′-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, maybe altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers can be used, for example, to stabilize 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides could be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

Other compounds that can be screened in accordance with these embodiments include but are not limited to small organic molecules that affect the expression of the Smad3 gene or some other gene balancing the interaction of intracellular proteins with Smad3 or the interaction of transmembrane proteins with Smad3 (e.g., by interacting with the regulatory region or transcription factors involved in gene expression); or such compounds that affect the activity of Smad3 or the activity of some other intracellular protein that interacts with Smad3 or of some other transmembrane protein that interacts with Smad3 or of promoters of target genes regulated by Smad3.

Computer modelling and searching technologies permit identification of compounds, or the improvement of already identified compounds, that can inhibit Smad3 expression or activity. Having identified such a compound or composition, the active sites or regions are identified. Such active sites might typically be ligand binding sites, such as the interaction domains of the ligand with Smad3 itself. The active site can be identified using methods known in the art including, for example, from the amino acid sequences of peptides, from the nucleotide sequences of nucleic acids, or from study of complexes of the relevant compound or composition with its ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined. Indeed, the Smad interaction domains have been determined for known inhibitors of Smad3, including the transcriptional repressors TGIF and SIP1, the adenoviral oncoprotein E1A, and the human oncogenes Ski, SnoN, and Evi-1 and may serve as the basis for rational drug design.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate inhibiting compounds can be identified by searching databases containing compounds along with information on their molecular structure. Such a search seeks compounds having structures that match the determined active site structure and that interact with the groups defining the active site. Such a search can be manual, but is preferably computer assisted. The compounds found from this search are potential Smad3 inhibiting compounds.

Alternatively, these methods can be used to identify improved inhibiting compounds from an already known inhibiting compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified inhibiting compounds or ligands of improved specificity or activity.

Further experimental and computer modeling methods useful to identify inhibiting compounds will be apparent to those of skill in the art based upon identification of the active sites of Smad3, and of intracellular and transmembrane proteins that interact with Smad3, and of related transduction and transcription factors, as well as of promoters of target genes regulated by Smad3.

Examples of molecular modelling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka, 1988 New Scientist 54-57; McKinaly and Rossmann, 1989 Annu Rev Pharmacol Toxiciol 29:111-122; Perry and Davies, OSAR: Quantitative Structure-Activity Relationships in Drug Design pp. 189-193 (Alan R. Liss, Inc. 1989); Lewis and Dean, 1989 Proc R Soc Lond 236:125-140 and 141-162; and, with respect to a model receptor for nucleic acid components, Askew, et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one could also screen libraries of known compounds, including natural products or synthetic chemicals, and biologically active materials, including proteins, for compounds that are inhibitors of Smad3.

Compounds identified via assays such as those described herein may be useful, for example, in elaborating the biological function of the Smad3 gene product, and for ameliorating disorders mediated by Smad3. Assays for testing the effectiveness of compounds identified by appropriate techniques are discussed below in the relevant sections. . . .

In Vitro Screening Assays for Compounds that Bind to Smad3

In vitro systems can be designed to identify compounds capable of interacting with (e.g., binding to) Smad3. Compounds identified are useful, for example, in inhibiting the activity of wild type and/or mutant Smad3 gene products; are useful in elaborating the biological function of Smad3; can be utilized in screens for identifying compounds that disrupt normal Smad3 interactions; or can in themselves disrupt such interactions.

The principle of the assays used to identify compounds that bind to Smad3 involves preparing a reaction mixture of Smad3 and the test compound under conditions and for a time sufficient to allow the two components to interact and bind, thus forming a complex that can be removed and/or detected in the reaction mixture. The Smad3 species used can vary depending upon the goal of the screening assay. For example, where compounds that bind and inhibit or mimic the inhibitors or mimic the ligands of Smad3 and bind to and “neutralize” Smad3 are sought, the full length Smad3 protein, a peptide corresponding to a domain or a fusion protein containing a Smad3 domain fused to a protein or polypeptide that affords advantages in the assay system (e.g., labeling, isolation of the resulting complex, etc.) can be utilized.

The screening assays can be conducted in a variety of ways. For example, one method to conduct such an assay would involve anchoring the Smad3 protein, polypeptide, peptide or fusion protein or the test substance onto a solid phase and detecting Smad3/test compound complexes anchored on the solid phase at the end of the reaction. In one embodiment of such a method, the Smad3 reactant can be anchored onto a solid surface, and the test compound, which is not anchored, can be labeled, either directly or indirectly.

In practice, microtiter plates are conveniently utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for Smad3 protein, polypeptide, peptide or fusion protein or the test compound to anchor any complexes formed in solution, and a labeled antibody specific for the other component of the possible complex to detect anchored complexes.

Alternatively, cell-based assays can be used to identify compounds that interact with Smad3. To this end, cell lines that express Smad3, or cell lines (e.g., COS cells, CHO cells, fibroblasts, etc.) that have been genetically engineered to express Smad3 (e.g., by transfection or transduction of Smad3 DNA) can be used. Interaction of the test compound with, for example, the Smad3 expressed by the host cell can be determined by comparison or competition with native ligand.

Assays for Intracellular or Transmembrane Proteins that Interact with the Smad3

Any method suitable for detecting protein-protein interactions may be employed for identifying transmembrane proteins or intracellular proteins that interact with Smad3. Among the traditional methods that may be employed are co-immunoprecipitation, crosslinking and co-purification through gradients or chromatographic columns of cell lysates or proteins obtained from cell lysates and the Smad3 protein to identify proteins in the lysate that interact with the Smad3 protein. For these assays, the Smad3 component used can be a full length Smad3 protein, a peptide corresponding to a domain of Smad3 or a fusion protein containing a domain of Smad3. Once isolated, such an intracellular or transmembrane protein can be identified and can, in turn, be used, in conjunction with standard techniques, to identify proteins with which it interacts. For example, at least a portion of the amino acid sequence of an intracellular or transmembrane protein that interacts with Smad3 can be ascertained using techniques well known to those of skill in the art, such as via the Edman degradation technique. (See, e.g., Creighton, 1983, “Proteins: Structures and Molecular Principles”, W.H. Freeman & Co., N.Y., pp. 34-49). The amino acid sequence obtained can be used as a guide for the generation of oligonucleotide mixtures that can be used to screen for gene sequences encoding such intracellular and transmembrane proteins. Screening can be accomplished, for example, by standard hybridization or PCR techniques. Techniques for the generation of oligonucleotide mixtures and the screening are well-known. (See, e.g., Ausubel et al., 1989, Current Protocols in Molecular Biology, Green Publishing Associates and Wiley Interscience, N.Y., and PCR Protocols: A Guide to Methods and Applications, 1990, Innis, M. et al., eds. Academic Press, Inc., New York.)

Additionally, methods can be employed that result in the simultaneous identification of genes, which encode the transmembrane or intracellular proteins interacting with Smad3. These methods include, for example, probing expression, libraries, in a manner similar to the well known technique of antibody probing of λgt11 libraries, using labeled Smad3 protein, or a Smad3 polypeptide, peptide or fusion protein, e.g., a Smad3 polypeptide or Smad3 domain fused to a marker (e.g., an enzyme, fluor, luminescent protein, or dye), or an Ig-Fc domain.

One method, which detects protein interactions in vivo, the two-hybrid system, is described in detail for illustration only and not by way of limitation. One version of this system has been described (Chien et al. 1991 PNAS USA 88:9578-9582) and is commercially available from Clontech (Palo Alto, Calif.). The assay identifies proteins that interact with Smad3, whether physiologically or pharmacologically.

Briefly, utilizing such a system, plasmids are constructed that encode two hybrid proteins: one plasmid consists of nucleotides encoding the DNA-binding domain of a transcription activator protein fused to a Smad3 nucleotide sequence encoding Smad3, a Smad3 polypeptide, peptide or fusion protein, and the other plasmid consists of nucleotides encoding the transcription activator protein's activation domain fused to a cDNA encoding an unknown protein, which has been recombined into this plasmid as part of a cDNA library. The DNA-binding domain fusion plasmid and the cDNA library are transformed into a strain of the yeast Saccharomyces cerevisiae that contains a reporter gene whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, Smad3 may be used as the bait gene product. Total genomic or cDNA sequences are fused to the DNA encoding an activation domain. This library and a plasmid encoding a hybrid of a bait Smad3 gene product fused to the DNA-binding domain are co-transformed into a yeast reporter strain, and the resulting transformants are screened for those that express the reporter gene. For example, and not by way of limitation, a bait Smad3 gene sequence, such as the open reading frame of Smad3 (or a domain of Smad3), can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait Smad3 gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait Smad3 gene-GAL4 fusion plasmid into a yeast strain, which contains a lacZ gene driven by a promoter, which contains GAL4 activation sequence. A cDNA encoded protein, fused to GAL4 transcriptional activation domain, that interacts with bait Smad3 gene product will reconstitute an active GAL4 protein and thereby drive expression of the HIS3 gene. Colonies, which express HIS3, can be detected by their growth on petri dishes containing semi-solid agar based media lacking histidine. The cDNA can then be purified from these strains, and used to produce and isolate the bait Smad3 gene-interacting protein using techniques routinely practiced in the art.

Assays for Compounds that Interfere with Smad3/Intracellular or Smad3/Transmembrane Macromolecule Interaction

The macromolecules that interact with Smad3 are referred to, for purposes of this discussion, as “binding partners”. These binding partners are likely to be involved in the Smad3 signal transduction pathway, and therefore, in the role of Smad3 in regulation of cellular processes. Therefore, it is desirable to identify compounds that interfere with or disrupt the interaction of such binding partners with Smad3 that may be useful in regulating the activity of Smad3 and control disorders associated with Smad3 activity.

The basic principle of the assay systems used to identify compounds that interfere with the interaction between Smad3 and its binding partner or partners involves preparing a reaction mixture containing the Smad3 protein, polypeptide, peptide or fusion protein and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the Smad3 moiety and its binding partner. Control reaction mixtures are incubated without the test compound or with a placebo. The formation of any complexes between the Smad3 moiety and the binding partner is then detected. The formation of a complex in the control reaction, but not in the reaction mixture containing the test compound, indicates that the compound interferes with the interaction of Smad3 and the interactive binding partner. Additionally, complex formation within reaction mixtures containing the test compound and normal Smad3 protein can also be compared to complex formation within reaction mixtures containing the test compound and a mutant Smad3. This comparison may be important in those cases wherein it is desirable to identify compounds that disrupt interactions of mutant but not normal Smad3 proteins, for example.

The assay for compounds that interfere with the interaction of Smad3 and binding partners can be conducted in a heterogeneous or homogeneous format. Heterogeneous assays involve anchoring either the Smad3 moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the Smad3 moiety and interactive binding partner. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the Smad3 moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment can be accomplished simply by coating the solid surface with a solution of the Smad3 gene product or binding partner and drying. Alternatively, an immobilized antibody specific for the species to be anchored may be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the Smad3 moiety and the interactive binding partner is prepared in which either the Smad3 or its binding partner is labeled, but the signal generated by the label is quenched due to formation of the complex (see, e.g., U.S. Pat. No. 4,109,496 by Rubenstein, which utilizes this approach for immunoassays). The addition of a test substance that competes with and displaces one of the species from the preformed complex will result in the generation of a signal above background. In this way, test substances that disrupt Smad3/binding partner interaction can be identified.

In a particular embodiment, a Smad3 fusion can be prepared for immobilization. For example, Smad3, or a peptide fragment, e.g., corresponding to a domain, can be fused to a glutathione-5-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-Smad3 fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the Smad3 gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-Smad3 fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the Smad3/binding partner interaction can be detected by adding the labeled antibody and measuring the radioactivity associated with the beads.

In another embodiment of the invention, these same techniques can be employed using peptide fragments that correspond to the binding domains of Smad3 and/or the interactive binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the interactive binding partner is obtained, short gene segments can be engineered to express peptide fragments of the protein, which can then be tested for binding activity and purified or synthesized.

For example, and not by way of limitation, a Smad3 gene product can be anchored to a solid material as described above, by making a GST-Smad3 fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-Smad3 fusion protein and allowed to bind. After washing away unbound peptides, labeled bound material, representing the interactive binding partner binding domain, can be eluted, purified, and analyzed for amino acid sequence by well-known methods. Peptides so identified can be produced synthetically or fused to appropriate facilitative proteins using recombinant DNA technology.

In one embodiment, the “binding partner” is Smad4, with which Smad3 heteroligomerizes upon receptor activation. In another embodiment, the “binding partner” is SARA (Smad anchor for receptor activation), which recruits the cytoplasmic signal transducer Smad3. In a further embodiment, the “binding partner” is the cognate DNA binding site for Smad3. Smad MH2 domains are the locus of Smad-dependent transcriptional activation activity, and are the site of protein-protein interactions responsible for oligomerization of Smad proteins as well as heteromerization with other transcription factors. Thus, in some embodiments, the MH2 domain of Smad3 is substituted for Smad3 itself in the assays described herein.

Assays for Identification of Compounds that Ameliorate Disorders Mediated by Smad3

Compounds including, but not limited to, binding compounds identified via assay techniques such as those described in the preceding sections, can be tested for the ability to ameliorate disorders mediated by Smad3. The assays described above can identify compounds that affect Smad3 activity (e.g., compounds that bind to Smad3, inhibit binding of a natural ligand, and either block activation (antagonists) or mimic inhibitors of activation (agonists), and compounds that bind to a natural ligand of Smad3 and neutralize ligand activity); or compounds that affect Smad3 gene activity (by affecting Smad3 gene expression, including molecules, e.g., proteins or small organic molecules, that affect or interfere with splicing events so that expression of the full length or a truncated form of Smad3 can be modulated). However, it should be noted that the assays described can also identify compounds that inhibit Smad3 signal transduction (e.g., compounds that affect upstream or downstream signalling events). The identification and use of such compounds that affect another step in the Smad3 signal transduction pathway in which the Smad3 gene and/or Smad3 gene product is involved and, by affecting this same pathway may modulate the effect of Smad3 on cellular processes are within the scope of the invention. Such compounds can be used as part of a method for the treatment of disorders mediated by Smad3.

Aspects of the invention also encompass cell-based and animal model-based assays for the identification of compounds exhibiting such an ability to ameliorate disorders mediated by Smad3.

Cell-based systems can be used to identify compounds that act to ameliorate disorders mediated by Smad3. Such cell systems can include, for example, recombinant or non-recombinant cells, such as cell lines, which express the Smad3 gene. For example monocyte cells, keratinocyte cells, or cell lines derived from monocytes or keratinocytes can be used. In addition, expression of host cells (e.g., COS cells, CHO cells, fibroblasts) genetically engineered to express a functional Smad3 and to respond to activation by the natural Smad3 ligand, e.g., as measured by a chemical or phenotypic change, induction of another host cell gene, amino acid phosphorlyation of host cell proteins, etc., can be used as an end point in the assay.

In utilizing such cell systems, cells are exposed to a compound suspected of exhibiting an ability to ameliorate a disorder mediated by Smad3, at a sufficient concentration and for a time sufficient to elicit a cellular phenotype associated with such an amelioration of a disorder mediated by Smad3 in the exposed cells. After exposure, the cells can be assayed to measure alterations in the expression of the Smad3 gene, e.g., by assaying cell lysates for Smad3 mRNA transcripts (e.g., by Northern analysis) or for Smad3 protein expressed in the cell; compounds that inhibit expression of the Smad3 gene are good candidates as therapeutics. Alternatively, the cells are examined to determine whether one or more cellular phenotype associated with a presentation of a disorder mediated by Smad3 has been altered to resemble a more normal or more wild type cellular phenotype associated with an amelioration of a disorder mediated by Smad3. Still further, the expression and/or activity of components of the signal transduction pathway of which Smad3 is a part, or the activity of Smad3 signal transduction pathway itself can be assayed.

For example, after exposure, the cell lysates can be assayed for the presence of host cell proteins, as compared to lysates derived from unexposed control cells. The ability of a test compound to inhibit expression of specific Smad3 target genes in these assay systems indicates that the test compound inhibits signal transduction initiated by Smad3 activation. The cell lysates can be readily assayed using a Western blot format; i.e., the host cell proteins are resolved by gel electrophoresis, transferred and probed using a anti-host cell protein detection antibody (e.g., an anti-host cell protein detection antibody labeled with a signal generating compound, such as radiolabel, fluor, enzyme, etc.). Alternatively, an ELISA format could be used in which a particular host cell protein is immobilized using an antibody specific for the target host cell protein, and the presence or absence of the immobilized host cell protein is detected using a labeled second antibody. In still another approach, amino acid phosphorylation of host cell proteins can be measured as an end point for Smad3 regulated signal transduction. In yet another approach, ion flux, such as calcium ion flux, can be measured as an end point for Smad3 stimulated signal transduction. In yet a further approach, assays for compounds that interfere with Smad3 binding to its cognate DNA binding site utilize specific reporter constructs, such as (SBE)4-luciferase reporter, driven by four repeats of the sequence identified as a Smad binding element in the JunB promoter.

In addition, animal-based systems may be used to identify compounds capable of ameliorating disorders mediated by Smad3. Such animal models may be used as test substrates for the identification of drugs, pharmaceuticals, therapies and interventions that may be effective in treating such disorders. For example, animal models can be exposed to a compound, suspected of exhibiting an ability to ameliorate a disorder mediated by Smad3, at a sufficient concentration and for a time sufficient to elicit such an amelioration of a disorder mediated by Smad3 in the exposed animals. The response of animals to the exposure can be monitored by assessing the reversal of disorders mediated by Smad3. With regard to intervention, any treatments that reverse any aspect of symptoms characteristic of disorders mediated by Smad3 should be considered as candidates for human therapeutic intervention in ameliorating disorders mediated by Smad3. Dosages of test agents may be determined by deriving dose-response curves, as discussed in the sections below.

Inhibition of Smad3 Expression or Smad3 Activity to Ameliorate Smad3 Mediated Disorders

The invention encompasses methods and compositions for modifying Smad3 regulated processes and treating Smad3 mediated disorders. Because a loss of normal Smad3 gene product results in the development of a desireable phenotype, a decrease in Smad3 gene product expression or activity, or deactivation of the Smad3 pathway, would facilitate progress towards a desireable state in individuals exhibiting a need for amelioration of Smad3 mediated disorders. Any approach that neutralizes Smad3 or inhibits expression of Smad3 (either transcription or translation) can be used to effectuate amelioration of disorders mediated by Smad3.

For example, the administration of peptides and analogues thereof, proteins, fusion proteins, carbohydrates, lipids, nucleic acid sequences such as aptamers, antibodies (including anti-idiotypic antibodies) and fragments thereof, and small organic compounds (e.g., peptidomimetics) and inorganic compounds that bind to Smad3, or to intracellular proteins that interact with Smad 3, or to transmembrane proteins that interact with Smad3 and inhibit the activity triggered by Smad3 or mimic the inhibitors of Smad3 can be used to ameliorate disorders mediated by Smad3. To this end, peptides corresponding to the cytoplasmic domain of the TGF-β or activin receptor (or a domain of a Smad involved in forming dimers with Smad3) can be utilized. Alternatively, anti-idiotypic antibodies or Fab fragments of antiidiotypic antibodies that mimic the cytoplasmic domain of the TGF-β or activin receptor (or the domain of a Smad involved in forming dimers with Smad3) and that neutralize Smad3 can be used. Such Smad3 peptides, proteins, fusions proteins, antibodies, anti-idiotypic antibodies or Fabs are administered to a subject in amounts sufficient to neutralize Smad3 and effectuate amelioration of disorders mediated by Smad3.

In some embodiments, the peptides, proteins, fusions proteins, antibodies, anti-idiotypic antibodies or Fabs are cell-permeable compounds. In other embodiments, cells are genetically engineered using recombinant DNA techniques to introduce the coding sequence for the peptide, protein, fusion protein, antibody, anti-idiotypic antibody or Fab into the cell, e.g., by transduction (using viral vectors, such as retroviruses, adenoviruses, and adeno-associated viruses) or transfection procedures, including but not limited to, the use of naked DNA or RNA, plasmids, cosmids, YACs, electroporation, liposomes, etc. The coding sequence can be placed under the control of a strong constitutive or inducible promoter, or a tissue-specific promoter, to achieve expression of the gene product. The engineered cells that express the gene product can be produced in vitro and introduced into the patient, e.g., systemically, intraperitoneally, at the site in the body, or the cells can be incorporated into a matrix and implanted in the body, e.g., genetically engineered cells can be implanted as part of a tissue or organ graft. Alternatively, the engineered cells that express the gene product can be produced following in vivo gene therapy approaches.

In other embodiments, monoclonal antibodies are produced in one of three different ways. They can be generated as mouse antibodies that are subsequently “humanized” by recombination with human antibody genes (Kohler and Milstein 1975 Nature 256:495; Winter and Harris 1993 Trends Pharmacol Sci 14:139; and Queen et al. 1989 PNAS USA 86:10029). Alternatively, human antibodies are raised in nude mice grafted with human immune cells (Bruggemann and Neuberger 1996 Immunol Today 8:391). Finally antibodies can also be made by phage display techniques (Huse et al. 1989 Science 246:1275; Hoogenboom et al. 1998 Immunotechnology 4:1; and Rodi and Makowski 1999 Curr Opin Biotechnol 10:87).

For the production of antibodies, various host animals may be immunized by injection with Smad3, a Smad3 peptide, functional equivalents or mutants of Smad3. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique of Kohler and Milstein (1975 Nature 256:495-497; and U.S. Pat. No. 4,376,110), the human B-cell hybridoma technique (Kosbor et al. 1983 Immunology Today 4:72; Cole et al. 1983 PNAS USA 80:2026-2030), and the EBV-hybridoma technique (Cole et al. 1985 Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb can be cultivated in vitro or in vivo. Production of high titers of mAbs in vivo is preferred.

In addition, techniques developed for the production of “chimeric antibodies” (Morrison et al. 1984 PNAS USA 81:6851-6855; Neuberger et al 1984 Nature 312:604-608; Takeda et al. 1985 Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird 1988 Science 242:423-426; Huston et al. 1988 PNAS USA 85:5879-5883; and Ward et al. 1989 Nature 334:544-546) can be adapted to produce single chain antibodies against Smad3 gene products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)2 fragments, which can be produced by pepsin digestion of the antibody molecule, and the Fab fragments, which can be generated by reducing the disulfide bridges of the F(ab′)2 fragments. Alternatively, Fab expression libraries may be constructed (Huse et al. 1989 Science 246:1275-1281) to allow rapid and easy identification of monoclonal Fab fragments with the desired specificity.

Antibodies to ligands of Smad3 can, in turn, be utilized to generate anti-idiotype antibodies that “mimic” these ligands, using techniques well known to those skilled in the art. (See, e.g., Greenspan & Bona 1993 FASEB J 7(5):437-444; and Nissinoff 1991 J Immunol 147(8):2429-2438). For example antibodies that bind to the cytoplasmic domain of the TGF-β or activin receptor (or the domain of a Smad involved in forming dimers with Smad3) and competitively inhibit the binding of Smad3 to the TGF-β or activin receptor (or a Smad involved in forming dimers with Smad3) can be used to generate anti-idiotypes that “mimic” these ligands and, therefore, bind and neutralize Smad3. Such neutralizing anti-idiotypes or Fab fragments of such anti-idiotypes can be used in therapeutic regimens to neutralize Smad3 and ameliorate disorders mediated by Smad3.

In alternate embodiments, interventions to ameliorate disorders mediated by Smad3 can be designed by reducing the level of endogenous Smad3 gene expression, e.g., using antisense, ribozyme, or interfering RNA approaches to inhibit or prevent translation of Smad3 mRNA transcripts; triple helix approaches to inhibit transcription of the Smad3 gene; or targeted homologous recombination to inactivate or “knock out” the Smad3 gene or its endogenous promoter. Delivery techniques are preferably designed for a systemic approach. Alternatively, the antisense, ribozyme or interfering RNA constructs described herein can be administered directly to the site containing the target cells.

Antisense approaches involve the design of oligonucleotides (either DNA or RNA) that are complementary to Smad3 mRNA. The antisense oligonucleotides will bind to the complementary Smad3 mRNA transcripts and ameliorate translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently shown to be effective at inhibiting translation of mRNAs as well. See generally, Wagner, R. 1994 Nature 372:333-335. Thus, oligonucleotides complementary to either the 5′- or 3′-non-translated, non-coding regions of Smad3 could be used in an antisense approach to inhibit translation of endogenous Smad3 mRNA. Oligonucleotides complementary to the 5′ untranslated region of the mRNA should include the complement of the AUG start codon. Antisense oligonucleotides complementary to mRNA coding regions can also be used in accordance with the invention. Whether designed to hybridize to the 5′-, 3′- or coding region of Smad3 mRNA, antisense nucleic acids should be at least six nucleotides in length, and are preferably oligonucleotides ranging from 6 to about 50 nucleotides in length. In specific aspects the oligonucleotide is at least 6 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to ameliorate specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), agents facilitating transport across the cell membrane (see, e.g., Letsinger et al. 1989 PNAS USA 86:6553-6556; Lemaitre et al. 1987 PNAS USA 84:648-652; PCT International Publication WO88/09810, published 1988) or other barriers, hybridization-triggered cleavage agents (See, e.g., Krol et al. 1988 BioTechniques 6:958-976) or intercalating agents (see, e.g., Zon 1988 Pharm Res 5:539-549). To this end, the oligonucleotide can be conjugated to another molecule, e.g., a peptide, hybridization triggered cross-linking agent, transport agent, hybridization-triggered cleavage agent, etc.

The antisense oligonucleotide can comprise at least one modified base moiety, which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The oligonucleotides described herein can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988 Nucl Acids Res 16:3209), methylphosphonate oligonucleotides can be prepared by use of controlled pore glass polymer supports (Sarin et al. 1988 PNAS USA 85:7448-7451), etc.

The antisense molecules can be delivered to cells that express the Smad3 protein in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous Smad3 transcripts and thereby prevent translation of the Smad3 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon 1981 Nature 290:304-310), the promoter contained in the 3′ long terminal repeat of Rous sarcoma virus (Yamamoto et al. 1980 Cell 22:787-797), the herpes thymidine kinase promoter (Wagner et al. 1981 PNAS USA 78:1441-1445), the regulatory sequences of the metallothionein gene (Brinster et al. 1982 Nature 296:39-42), etc. Any type of plasmid, cosmid, YAC or viral vector can be used to prepare the recombinant DNA construct, which can be introduced directly into the tissue site. Alternatively, viral vectors can be used that selectively infect the desired tissue, in which case administration may be accomplished by another route (e.g., systemically).

Ribozyme molecules-designed to catalytically cleave Smad3 mRNA transcripts can also be used to ameliorate translation of Smad3 mRNA and expression of Smad3. (See, e.g., PCT International Publication WO90/11364, published 1990; Sarver et al. 1990 Science 247:1222-1225). While ribozymes that cleave mRNA at site specific recognition sequences can be used to destroy Smad3 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach 1988 Nature 334:585-591. There are a plurality of potential hammerhead ribozyme cleavage sites within the nucleotide sequence of human Smad3 cDNA. Preferably the ribozyme is engineered so that the cleavage recognition site is located near the 5′ end of the Smad3 mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al. 1984 Science 224:574-578; Zaug and Cech 1986 Science 231:470-475; Zaug, et al. 1986 Nature 324:429-433; PCT International Publication No. WO 88/04300 published 1988; Been and Cech 1986 Cell 47:207-216). The Cech-type ribozymes have an eight base pair active site, which hybridizes to a target RNA sequence whereafter cleavage of the target RNA takes place. Aspects of the invention encompass those Cech-type ribozymes that target eight base-pair active site sequences that are present in Smad3.

As in the antisense approach, the ribozymes can be composed of modified oligonucleotides (e.g. for improved stability, targeting, etc.) and should be delivered to cells that express Smad3 in vivo. A preferred method of delivery involves using a DNA construct “encoding” the ribozyme under the control of a strong constitutive pol III or pol II promoter, so that transfected cells will produce sufficient quantities of the ribozyme to destroy endogenous Smad3 messages and inhibit translation. Because ribozymes unlike antisense molecules, are catalytic, a lower intracellular concentration is required for efficiency.

RNA interference refers to the process of sequence-specific post-transcriptional gene silencing in animals mediated by short interfering RNAs (siRNAs) (Fire et al. 1998 Nature 391:806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes which is commonly shared by diverse flora and phyla (Fire et al. 1999 Trends Genet 15:358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al. 2001 Nature 409:363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNAs) from precursor RNA of conserved structure that are implicated in translational control (Hutvagner et al. 2001 Science 293:834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al. 2001 Genes Dev 15:188).

Short interfering RNA mediated RNAi has been studied in a variety of systems. Fire et al. 1998 Nature 391:806, were the first to observe RNAi in C. elegans. Wianny and Goetz 1999 Nature Cell Biol 2:70, describe RNAi mediated by dsRNA in mouse embryos. Hammond et al. 2000 Nature 404:293, describe RNAi in Drosophila cells transfected with dsRNA. Elbashir et al. 2001 Nature 411:494, describe RNAi induced by introduction of duplexes of synthetic 21-nucleotide RNAs in cultured mammalian cells including human embryonic kidney and HeLa cells. Recent work in Drosophila embryonic lysates (Elbashir et al. 2001 EMBO J. 20:6877) has revealed certain requirements for siRNA length, structure, chemical composition, and sequence that are essential to mediate efficient RNAi activity. These studies have shown that 21-nucleotide siRNA duplexes are most active when containing 3′-terminal di-nucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end o of the siRNA guide sequence (Elbashir et al. 2001 EMBO J. 20:6877). Other studies have indicated that a 5′-phosphate on the target-complementary strand of a siRNA duplex is required for siRNA activity and that ATP is utilized to maintain the 5′-phosphate moiety on the siRNA (Nykanen et al. 2001 Cell 107:309).

Endogenous Smad3 gene expression can also be reduced by inactivating or “knocking out” the Smad3 gene or its promoter using targeted homologous recombination. (E.g., see Smithies et al. 1985 Nature 317:230-234; Thomas & Capecchi 1987 Cell 51:503-512; Thompson et al. 1989 Cell 5:313-321). For example, a mutant, non-functional Smad3 protein (or a completely unrelated DNA sequence) flanked by DNA homologous to the endogenous Smad3 gene (either the coding regions or regulatory regions of the Smad3 gene) can be used, with or without a selectable marker and/or a negative selectable marker, to transfect cells that express Smad3 in vivo. Insertion of the DNA construct, via targeted homologous recombination, results in inactivation of the Smad3 gene. This approach is acceptable for use in humans provided the recombinant DNA constructs are directly administered or targeted to the required site using appropriate viral vectors.

Alternatively, endogenous Smad3 gene expression can be reduced by targeting deoxyribonucleotide sequences complementary to the regulatory region of the Smad3 gene (i.e., the Smad3 promoter and/or enhancers) to form triple helical structures that prevent transcription of the Smad3 gene in target cells in the body. (See generally, Helene, C. 1991 Anticancer Drug Des 6(6):569-84; Helene, C. et al. 1992 Ann NY Acad Sci 660:27-36; and Maher, L. J. 1992 Bioassays 14(12):807-15).

In yet another embodiment, the activity of Smad3 can be reduced using a “dominant negative” approach. To this end, constructs that encode defective Smad3 proteins, can be used in gene therapy approaches to diminish the activity of Smad3 in appropriate target cells. For example, nucleotide sequences that direct host cell expression of Smad3 in which a domain or portion of a domain is deleted or mutated can be introduced into cells at appropriate target sites (by gene therapy methods described above). Alternatively, targeted homologous recombination can be utilized to introduce such deletions or mutations into the subject's endogenous Smad3 gene at appropriate target sites. The engineered cells will express non-functional Smad3 (i.e., a Smad 3 that is capable of binding its natural ligand, but incapable of signal transduction). Such engineered cells at appropriate target sites should demonstrate a diminished activation of downstream events and a heightened response to TGF-βs and possibly activins.

Pharmaceutical Preparations and Methods of Administration

The compounds that are determined to affect Smad3 gene expression or Smad3 activity can be administered to a patient at therapeutically effective doses. A therapeutically effective dose refers to that amount of the compound sufficient to result in amelioration of symptoms of Smad3 mediated disorders. The compounds of the invention are generally administered to animals, including humans.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It will be appreciated that the actual preferred amounts of active compound in a specific case will vary according to the specific compound being utilized, the particular compositions formulated, the mode of application, and the particular situs and organism being treated. Dosages for a give host can be determined using conventional considerations, e.g., by customary comparison of the differential activities of the subject compounds and of a known agent, e.g., by means of an appropriate, conventional pharmacological protocol.

Formulation and Use

The pharmacologically active compounds of this invention can be processed in accordance with conventional methods of galenic pharmacy to produce medicinal agents for administration to patients, e.g., mammals including humans.

The compounds of this invention can be employed in admixture with conventional excipients, i.e., pharmaceutically acceptable organic or inorganic carrier substances suitable for parenteral, enteral (e.g., oral) or topical application, which do not deleteriously react with the active compounds. Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules. A syrup, elixir, or the like can be used wherein a sweetened vehicle is employed.

Sustained or directed release compositions can be formulated, e.g., by inclusion in liposomes or incorporation into an epidermal patch with a suitable carrier, for example DMSO. It is also possible to freeze-dry these compounds and use the lyophilizates obtained, for example, for the preparation of products for injection.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLE 1

### Smad3 Signaling is Required for Epithelial-Mesenchymal Transition of Lens Epithelium Post-Injury

Lens epithelial cells undergo epithelial-mesenchymal transition (EMT) following injury as in cataract extraction, leading to fibrosis of the lens capsule. Fibrosis of the anterior capsule can be modeled in the mouse by capsular injury in the lens which results in EMT of the lens epithelium and subsequent deposition of extracellular matrix without contamination of other cell types from outside the lens. We have previously shown that signaling via Smad3, a key signal transducing element downstream of TGF-β and activin receptors, is activated in lens epithelial cells by 12 hr post-injury and that this Smad3 activation is blocked by administration of a TGF-β2-neutralizing antibody in mice. We now show that EMT of primary lens epithelial cells in vitro depends on TGF-β expression and that injury-induced EMT in vivo depends, more specifically, on signaling via Smad3. Loss of Smad3 in mice blocks both morphological changes of lens epithelium to a mesenchymal phenotype and expression of the EMT markers snail, α-smooth muscle actin, lumican and type I collagen in response to injury in vivo or to exposure to exogenous TGF-β in organ-culture. The results indicate that blocking the Smad3 pathway would be beneficial in inhibiting post-injury/-surgery capsular fibrosis.

Introduction

Certain cells have an inherent plasticity such that their morphology and phenotype can be modulated by various growth factors and extracellular stimuli. As an example, the ability of an epithelial cell to change its morphology and its transcriptional program to that characteristic of a mesenchymal cell, or so-called epithelial-mesenchymal transition (EMT), is important not only in development, but also in wound healing, fibrosis, and invasion and metastasis of tumor cells (Hay, E. D. and Zuk, A. 1995 Am J Kidney Dis 26: 678-690; Hay, E. D. 1995 Acta Anat (Basel) 154: 8-20; Savagner, P. 2001 Bioessays 23: 912-923).

Although lens epithelial cells are derived from surface ectoderm, they express vimentin (Sax et al. 1990 Dev Biol 139:56-64) as well as the epithelial surface marker, N-cadherin (Xu, L. et al. 2002 Exp Eye Res 74: 753-760). Transdifferentiation of these cells into elongated mesenchymal-like cells involves transcriptional reprogramming as evidenced by expression of type I collagen and α-smooth muscle actin (αSMA) (Zuk, A. and Hay, E. D. 1994 Dev Dyn 201: 378-393; Marcantonio, J. M. and Vrensen, G. F. 1999 Eye 13: 484-488; Hales, A. M. et al. 1994 Curr Eye Res 13: 885-890; Saika, S. et al. 1998 Exp Eye Res 66: 283-294). This well-established EMT is readily observed post-injury in vivo or in cell culture. EMT in these cells in vivo results in fibrosis and/or contraction of the capsular tissue (Zuk, A. and Hay, E. D. 1994 Dev Dyn 201: 378-393; Saika, S. et al. 1998 Exp Eye Res 66: 283-294). Similar injury-induced EMT is observed following cataract surgery, although in this operation the entire lens content is removed and the cells migrate to the posterior capsular surface resulting in fibrosis of the posterior capsule as well as the residual anterior capsule (Saika, S. et al. 1998 Exp Eye Res 66: 283-294; Saika, S. et al. 2001 Exp Eye Res 72: 679-686; Saika, S. et al. 2002 Br J Ophthalmol 86: 1428-1433; Saika, S. et al. 2003 Invest Ophthalmol Vis Sci 44: 2094-2102). The resultant fibrosis, referred to as post-operative capsular opacification, can impair patients' vision. Animal lenses are exceptionally suitable for detailed analysis of EMT in vivo, because the lens contains only one epithelial cell type and there is little chance of contamination with other cells post-injury. A puncture wound in the anterior capsule of a mouse lens is sealed by fibrotic tissue, containing αSMA-positive fibroblastic-like lens cells (Saika, S. et al. 2001 Exp Eye Res 72: 679-686; Saika, S. et al. 2002 Br J Ophthalmol 86: 1428-1433; Saika, S. et al. 2003 Invest Ophthalmol Vis Sci 44: 2094-2102).

Growth factors, including especially transforming growth factor-β (TGF-β), orchestrate the EMT of various epithelial tissues in response to injury (Hay, E. D. and Zuk, A. 1995 Am J Kidney Dis 26: 678-690; Hay, E. D. 1995 Acta Anat (Basel) 154: 8-20; Savagner, P. 2001 Bioessays 23: 912-923; Moustakas, A. et al. 2002 Immunol Lett 82: 85-91; ten Dijke, P. et al. 2002 J Cell Physiol. 191: 1-16). TGF-β2 is a likely mediator of EMT in lens epithelial cells in vivo, because it is expressed at much higher levels than the other TGF-β isoforms in the aqueous humor which bathes the lens tissue (Jampel, H. D. et al. 1990 Curr Eye Res 9: 963-969), as well as in the vitreous (Connor, T. B. Jr. et al. 1989 J Clin Invest 83: 1661-1666). TGF-β2 also up-regulates αSMA in lens epithelial cells in vitro and in organ-culture (Kurosaka, D. et al. 1995 Invest Ophthalmol Vis Sci 1995, 36: 1701-1708). TGF-β signals through a pair of transmembrane receptor serine-threonine kinases and downstream mediators called Smad proteins. Receptor-activated Smad proteins, Smad2 and Smad3, are phosphorylated directly by the TβRI receptor kinase, partner with the common mediator, Smad4, and translocate to the nucleus where they play a prominent role in activation of TGF-β-dependent gene targets (ten Dijke, P. et al. 2002 J Cell Physiol. 191:1-16; Massague, J and Wotton, D. 2000 EMBO J. 19: 1745-1754). Despite the importance of this pathway in mediating transcriptional effects of TGF-β on cells (Piek, E. et al. 2001 J Biol Chem 276: 19945-19953; Verrecchia, F and Mauviel, A. 2002 J Invest Dermatol 118: 211-215), its role in mediating EMT is controversial (Piek, E. et al. 1999 J Cell Sci 112: 4557-4568; Yu, L. et al. 2002 EMBO J 21: 3749-3759; Oft, M. et al. 2002 Nat Cell Biol 4: 487-494; Bakin, A. V. et al. 2000 J Biol Chem 275: 36803-36810; Bhowmick, N. A. et al. 2001 Mol Biol Cell 12: 27-36; Janda, E. et al. 2002 J Cell Biol 156: 299-314; Oft, M. et al. 1996 Genes Dev 10: 2462-2477; Bhowmick, N. A. et al. 2001 J Biol Chem 276: 46707-46713; Itoh, S. et al. 2003 J Biol Chem 278: 3751-3761). Such studies are based on use of a relatively limited number of cell lines in vitro, and none have addressed the role of Smad signaling in EMT in vivo, in processes such as response to injury. We have previously reported that activation of Smad3/4 signaling in lens epithelial cells post-capsular injury was blocked by an injection of neutralizing antibody to TGF-β2 in mice (Saika, S. et al. 2001 Exp Eye Res 72: 679-686), indicating that injury-induced Smad3/4 signaling is likely to be mediated by TGF-β2. A similar nuclear translocation of Smad3/4 is observed post-cataract surgery in human lens epithelial cells (Saika, S. et al. 2002 Br J Ophthalmol 86: 1428-1433). These findings led us to hypothesize that injury-induced EMT of lens epithelium is initiated by activation of TGF-β/Smad3 signaling.

In the present study, we have directly addressed the role of TGF-β/Smad3 signaling in EMT of lens epithelial cells both in vitro and in vivo. We use a TGF-β neutralizing antibody, to show that endogenous TGF-β is involved in the initiation of EMT in primary porcine lens epithelial cells in vitro. Most importantly, we have utilized Smad3^{ex8/ex8} (KO) mice (Yang, X. et al. 1999 EMBO J. 18: 1280-1291) to show that EMT of lens epithelium post-injury in vivo is completely blocked in the absence of Smad3, consistent with the absence of expression of EMT markers including, snail (Carver, E. A. et al. 2001 Mol Cell Biol 21: 8184-8188), lumican, αSMA, and collagen seen in eyes of Smad3^{+/+} (WT) littermates. Together these results indicate that Smad3 is required for injury-induced EMT in lens epithelium and that inhibition of this pathway would be desirable clinically to prevent capsular opacification, which can be a complication of cataract surgery (Marcantonio, J. M. and Vrensen, G. F. 1999 Eye 13: 484-488).

Materials and Methods

All the experimental procedures were approved by Animal Care and Use Committee of the National Cancer Institute, National Institutes of Health, Bethesda, Md., and that of Wakayama Medical University, Wakayama, Japan, and conducted in accordance with the ARVO Statement for the Use of Animals in Ophthalmic and Vision Research.

EMT of Primary Culture of Porcine Lens Epithelial Cells.

Anterior lens capsules with an epithelial layer, obtained from a pig eye, were put in a 30-mm collagen-coated plastic culture dish to allow the epithelial cells to outgrow. After reaching confluence, the cells were trypsinized, suspended in Dulbecco's modified Eagle's medium (DMEM) supplemented with 10% fetal calf serum, and seeded on fibronectin-coated chamber slides (Falcon, Becton Dickinson, Lincoln Park, N.J.). Twenty four hrs later fresh culture medium supplemented with either 20 μg/ml of monoclonal pan-specific TGF-β-neutralizing antibody (R & D Systems, Minneapolis, Minn.) or bovine serum albumin was added and the cells were incubated for an additional 36 hrs. Cells were then fixed with 4% paraformaldehyde in 0.1 M phosphate buffer, processed for immunocytochemistry for αSMA as described below, and mounted in balsam. The percentage of αSMA-positive cells was determined by scoring expression in 100 cells taken from 3 independent areas.

For Western blotting for αSMA, passage 2 primary lens epithelial cells were grown until subconfluent in two 25 cm² fibronectin-coated culture bottles (Iwaki Glass, Tokyo, Japan) in culture medium supplemented with 10% fetal calf serum. They were then further incubated in serum-free DMEM with either 20 μg/ml of monoclonal pan-specific TGF-β-neutralizing antibody (R & D Systems) or non-immune IgG at the same concentration for an additional 72 hrs. The cells were scraped, collected and immediately mixed with 2× sample buffer. Proteins were separated by SDS-PAGE, transferred to PVDF membranes (Immobilon-P, Millipore, Bedford, Mass.), and blocked in 5% skim milk in phosphate-buffered saline (PBS). After incubation with primary antibodies against αSMA (1:500 dilution in PBS, Neomarker, UK, clone: 1A4) and actin (1:500 dilution in PBS, Santa Cruz Biochemicals, Santa Cruz, Calif.) at 4° C. overnight, blots were reacted with peroxidase-conjugated secondary antibodies and developed with ECL (Amersham Biosciences, Buckingshire, UK).

Subcapsular Injury in Mouse Eyes.

Adult Smad3-KO and WT mice (4-6 week-old, 72 KO and 72 WT mice) were anesthetized with an intraperitoneal injection of pentobarbital sodium (70 mg/Kg). (Yang, X. et al. 1999 EMBO J. 18: 1280-1291). A small incision was made in the central anterior capsule with the blade part of a 26 G hypodermic needle through a corneal incision in right eye after topical application of mydriatics and oxybuprocaine eyedrop as anesthetic. The left eye served as uninjured control. The depth of injury was approximately 300 μm or about one-fourth of the length of the blade part of the needle which we have reported previously leads to the formation of fibrotic tissue around the capsular break. (Saika, S. et al. 2003 Invest Ophthalmol Vis Sci 44: 2094-2102). After instillation of ofloxacin ointment, the animals were allowed to heal for 6 hrs to 8 weeks. Proliferating cells were labeled by an intraperitoneal injection of bromo-deoxyuridine (BrdU); mice were killed 2 hours later by CO₂ asphyxia and cervical dislocation and each eye was enucleated. Each timepoint is represented by 6 mice of each genotype; eyes of each genotype (both injured and uninjured controls) were fixed and embedded in paraffin.

Lens Capsular Explant Culture.

Ten-day old WT (n=7) and KO (n=5) mice from two litters were killed as described above and both lenses were enucleated. The lens capsule was carefully dissected and placed on fibronectin-coated chamber slides (Falcon, Becton Dickinson). The explants were incubated in DMEM-10% fetal calf serum for 12 days to allow the lens epithelial cells to grow out from the explanted lens. The maximum distance of outgrowth of the epithelial cell sheet from capsular specimen was measured and compared between WT and KO specimens to evaluate cell migratory activity. After fixation in 4% paraformaldehyde in 0.1 M phosphate buffer for 24 hrs, the capsule was removed from the chamber and processed for immunofluorescence staining for αSMA. For western blotting of explanted specimens for αSMA, lens capsules obtained from 10-day old mice were incubated as above for either for 6 (4 WT and 3 KO specimens) or 12 (4 WT and 4 KO specimens) days in a 12-well culture plate (Corning/Iwaki Glass, Corning, N.Y.). The cells and explanted capsular specimens were mixed in 2× sample buffer and processed for western blotting for αSMA as described above.

Organ-Culture of Lenses.

The crystalline lens was carefully removed from enucleated eyes of adult Smad3-KO or WT mice and processed for organ-culture as previously described (Saika, S. et al. 2003 Invest Ophthalmol Vis Sci 44: 2094-2102). Three lenses were used in each culture condition. The lens was incubated in DMEM supplemented with antibiotics in the presence and absence of porcine TGF-β2 (10 ng/ml) with a medium change every 2 days. After 5 or 10 days of culture, the tissue was fixed in 2.0% paraformaldehyde as described above. Paraffin sections were processed for histology and immunohistochemistry.

Histology and Immunohistochemistry.

Sections (5 μm) were deparaffinized and stained with hematoxylin and eosin (HE) alone or with polyclonal antibodies against collagen types I and V (both 1:100 dilution in PBS, Southern Biotechnology, Birmingham, Ala.), rabbit polyclonal anti-lumican antibody (10 μg/ml)(Saika, S. et al. 2000 J Biol Chem 275: 2607-2612), or with a mouse monoclonal anti-αSMA antibody (1:100 dilution in PBS, NeoMarker, Fremont, Calif., USA), rabbit polyclonal antibodies against the TGF-β isoforms as previously reported (Flanders, K. C. et al. 1991 Development 113: 183-191), or with non-immune IgG (control). After binding of labeled secondary antibody and the color reaction with 3,3′-diaminobenzidine, sections were counterstained with methyl green and mounted in balsam. For the explant experiments, cells were processed for immunofluorescence staining for αSMA and mounted in VectaShield with DAPI nuclear staining (Vector Laboratories, Burlingame, Calif.).

In Situ Hybridization for Snail and αSma mRNAs.

Digoxigenin-labeled riboprobes for mouse snail and αSma were prepared as previously reported using a digoxigenin labeling kit (Roche Diagnostics Corp-Boehringer Mannheim, Indianapolis) (Saika, S. et al. 2000 J Biol Chem 275: 2607-2612). In brief, digoxigenin-11-UTP-labeled single strand sense and antisense riboprobes were prepared from PCR products obtained from plasmids containing cDNA inserts for complete mouse snail (Gong, Y. et al. 2001 Cell 107: 513-523) or αSma mRNAs. PCR primers were as follows; 5′-CTGCTCTGCCTCTAGCACAC-3′ (SEQ ID NO: 1) and 5′-TTAAGGGTAGCACATGTCTG-3′ (SEQ ID NO: 2) for αSma and 5′-ACACTGGTGAGAAGCCATTC-3′ (SEQ ID NO: 3) and 5′-AGTTCTATGGCTCGAAGCAG-3′ (SEQ ID NO: 4) for snail. Paraffin sections 5 μm thick were subjected to the Ventana HX system of in situ hybridization (Ventana Medical Systems, Inc., Tucson) according to the manufacturer's protocol. In brief, paraffin sections were deparaffinized and digested with proteinase K (Ventana) at 37° C. for 2 min. After hybridization, sections were washed 3 times in 0.1×SSC high stringency solution (Ventana) at 65° C. for 3 times. Probes were detected with alkaline phosphatase-conjugated anti-digoxigenin-antibody of Fab fragments (Roche) at 37° C. for 30 min. Sections were removed from the system and color developed in freshly prepared substrate solution NBT-BCIP (DIG nucleic acid detection kit). Slides were counterstained with nuclear-red.

Results

EMT of Lens Epithelial Cells In Vitro Depends on TGF-β.

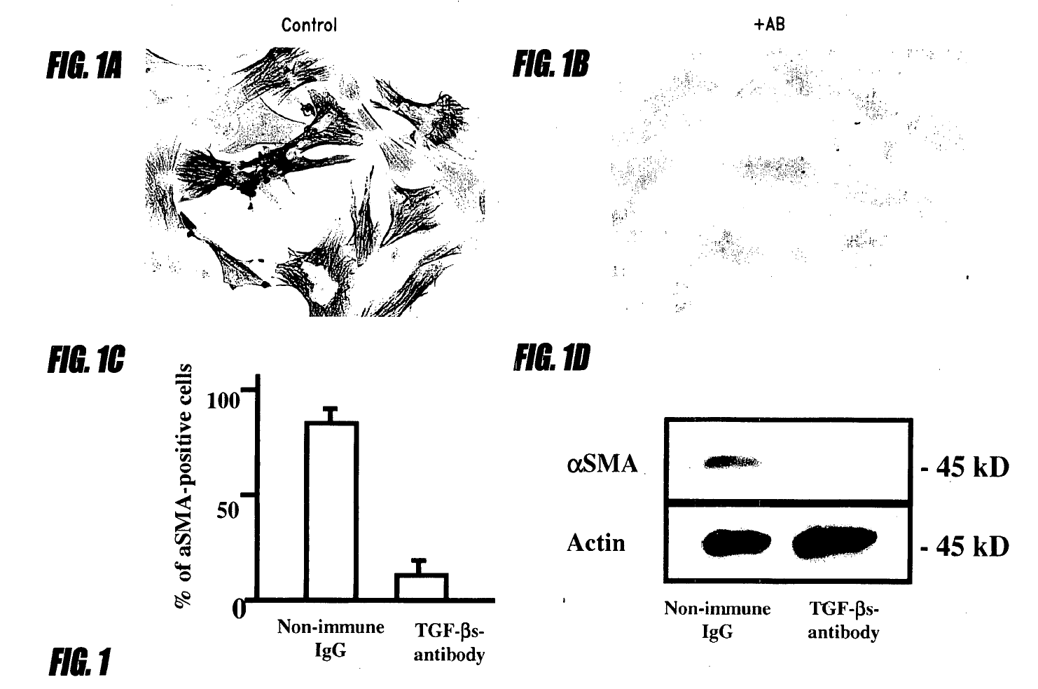

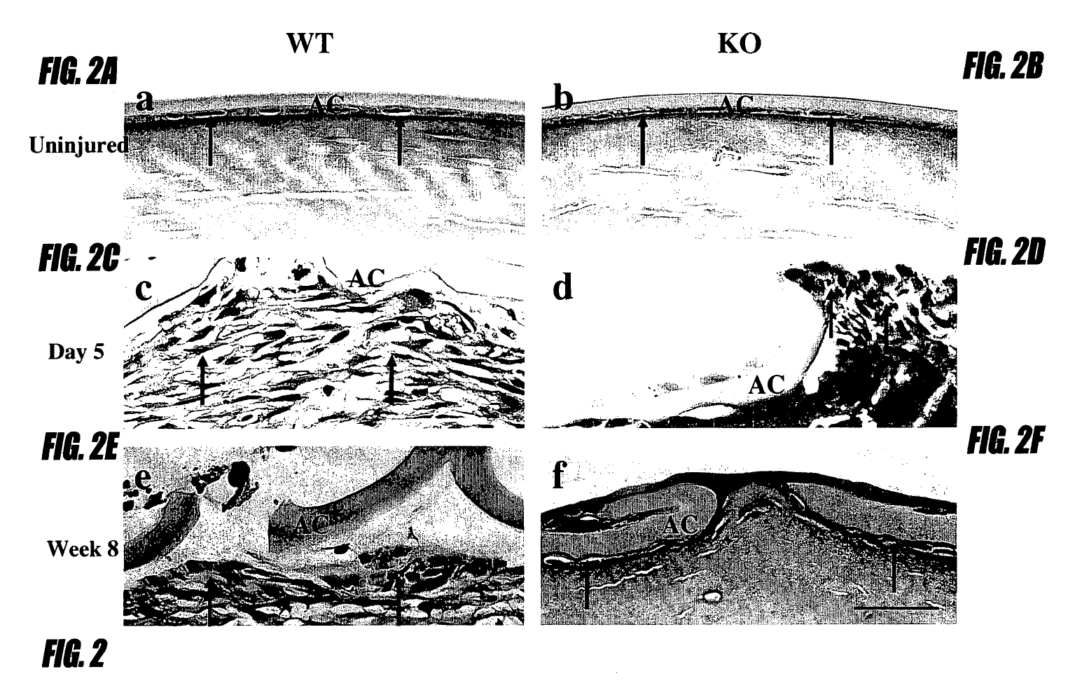

Primary porcine lens epithelial cells exhibited a fibroblast-like morphology and expressed αSMA, an established marker for EMT in lens epithelial cells (Marcantonio, J. M. and Vrensen, G. F. 1999 Eye 13: 484-488; Hales, A. M. et al. 1994 Curr Eye Res 13: 885-890; Saika, S. et al. 1998 Exp Eye Res 66: 283-294), 48 hr after culturing on fibronectin-coated chamber slides (FIG. 1a). Addition of 20 μg/ml of pan-specific neutralizing antibodies to TGF-β suppressed the up-regulation of αSMA as revealed by immunocytochemistry (FIG. 1b). Whereas 88+/−6.1% of the cells expressed αSMA in the absence of antibody, only 11+/−6.6% of the cells showed detectable staining following incubation with anti-TGF-β for 48 hrs (FIG. 1c). This was further confirmed by Western blotting of lysates of cells grown in serum-free medium for αSMA (FIG. 1d). Together these data indicate that TGF-β expressed by the lens epithelial cells stimulates cells to undergo EMT, as indicated by expression of the marker αSMA.

Histology of Injured Lenses of Smad3-Knockout Mice.

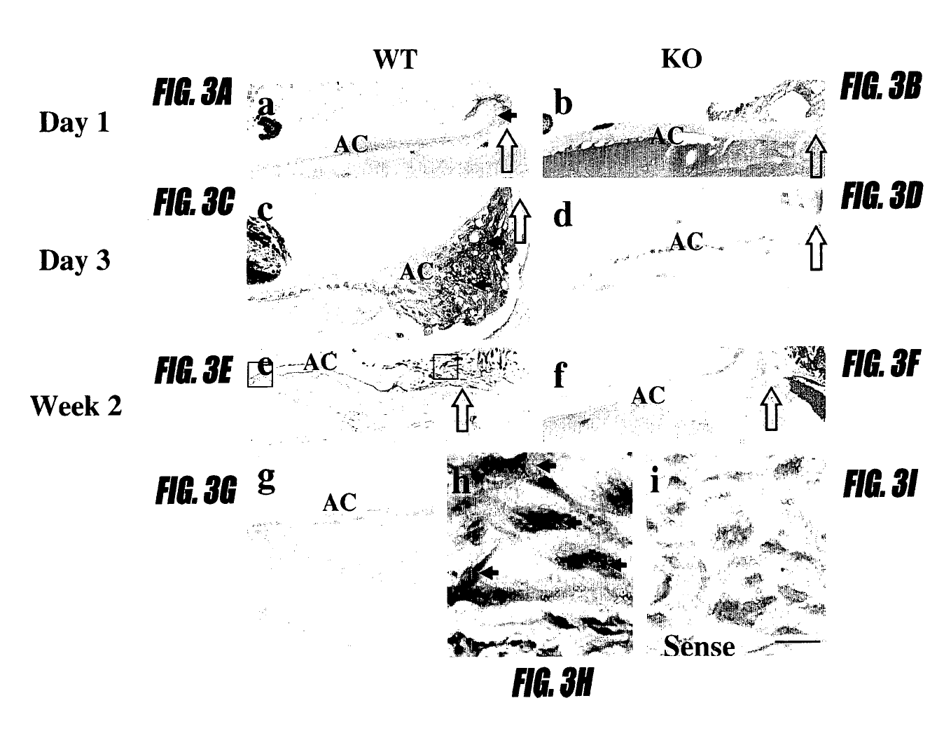

We have previously shown that lens epithelial cells in vivo undergo EMT by demonstrating acquisition of a fibroblastic morphology and expression of αSMA, an established marker for EMT in this cell type, following nuclear translocation of Smads3/4 in response to capsular injury in mice (Saika, S. et al. 2001 Exp Eye Res 72: 679-686; Saika, S. et al. 2003 Invest Ophthalmol Vis Sci 44: 2094-2102). In this study, nuclear translocation of Smad3/4 was detected within 12 hr post-injury, whereas expression of αSMA protein was not detected until 5 days (Saika, S. et al. 2001 Exp Eye Res 72: 679-686). To determine whether Smad3 might actually be required for this injury-induced EMT of lens epithelium, we injured the lenses of Smad3-null mice (KO) and littermate wildtype (WT) mice and examined the response at different times post-injury ranging from 1 day to 8 weeks. Lens epithelium of uninjured eyes (FIG. 2a, b) and injured eyes of KO and WT littermate controls exhibited a similar histology for the first 3 days post-injury, but striking differences were observed at later times (FIG. 2c-f). The break in the anterior capsule of WT eyes was sealed by an accumulation of multilayered lens-derived cells with a fibroblast-like morphology at 5 days post-injury (FIG. 2c). A similar accumulation of cells was not observed in KO eyes (FIG. 2d). Instead, the cells populating the wound area retained more of an epithelial-like morphology (FIG. 2d, f) compared to the elongated, fibroblast-like cells seen in WT specimens through week 8 (FIG. 2c, e). Notably, the morphology of KO cells at week 8 was similar to the cells of a normal, uninjured, lens (Arrows, FIG. 2f). In contrast, lens epithelial cells of Smad3 heterozygous mice displayed a morphology similar to that of WT mice at Day 5 post-injury. These histological findings indicated that loss of Smad3 blocks injury-induced EMT in lens epithelial cells in mice.

Expression of Snail in Injured Lens is Dependent on Smad3.

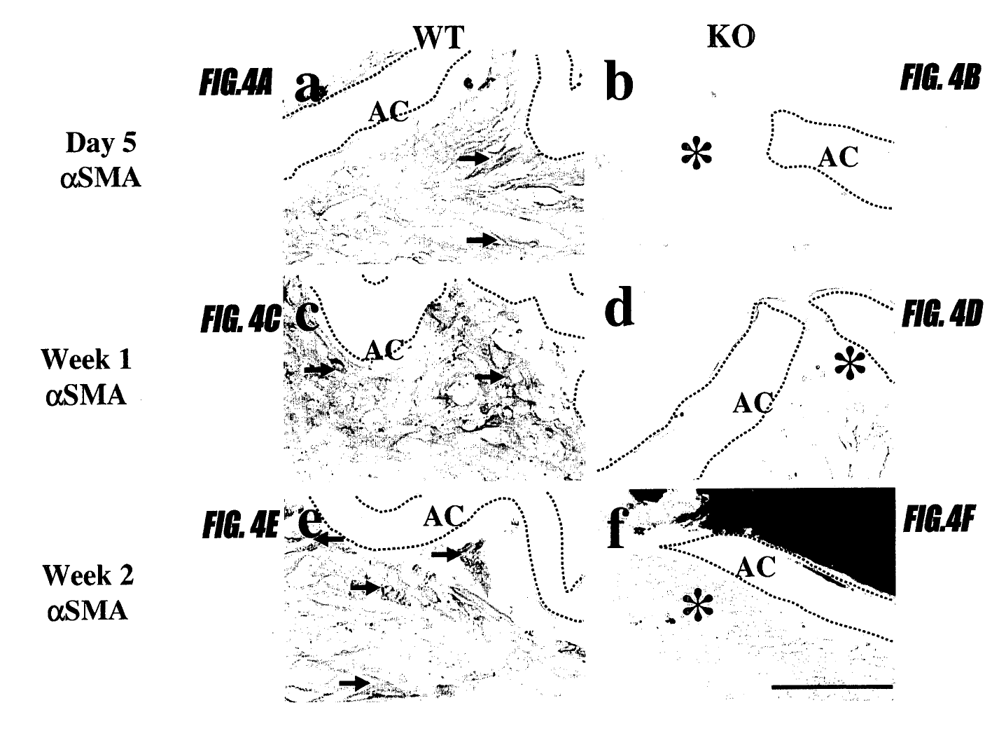

To further document the apparent block in EMT of lens epithelium seen in KO mice post-injury, we examined the expression of both early and later markers of EMT. Snail is a member of a family of zinc finger-containing transcriptional repressors increasingly associated with suppression of the epithelial phenotype associated with EMT (Savagner, P. 2001 Bioessays 23: 912-923) and shown to be an immediate-early Smad3-dependent gene target of TGF-β in fibroblasts (Yang, Y. C. et al. 2003 PNAS USA 100: 10269-10274). We therefore examined whether snail might also be an early marker of Smad3-dependent EMT in vivo. Whereas in situ hybridization for snail mRNA showed undetectable expression in uninjured WT or KO lenses, a signal could be seen in epithelial cells around the capsular break in WT lenses at 1 day post-injury (FIG. 3a, arrow) and at day 3 in the multilayer fibroblast-like cells (FIG. 3c, arrows). While fibroblast-like cells around the capsular break continued to express snail at later times post-injury (FIG. 3e, h, arrows), epithelial cells distal to the injury site were not labeled at this (FIG. 3e, g) or at earlier times post-injury (FIG. 3a, c). Lens epithelial cells in injured KO eyes never expressed snail mRNA (FIG. 3b, d, f), further supporting the notion that Smad3 signaling is required for EMT of lens epithelial cells. No signal was seen with the sense riboprobe (FIG. 3i).

Expression of Markers of the Later Stages of EMT in the Injured Lens is also Dependent on Smad3.

Since the data from histological analysis and in situ hybridization for snail were indicative of perturbed EMT in KO lens epithelium in response to injury, we examined if loss of Smad3 would also block expression of other markers characteristic of later stages of lens epithelial cell EMT such as lumican, αSMA, and collagen type I.

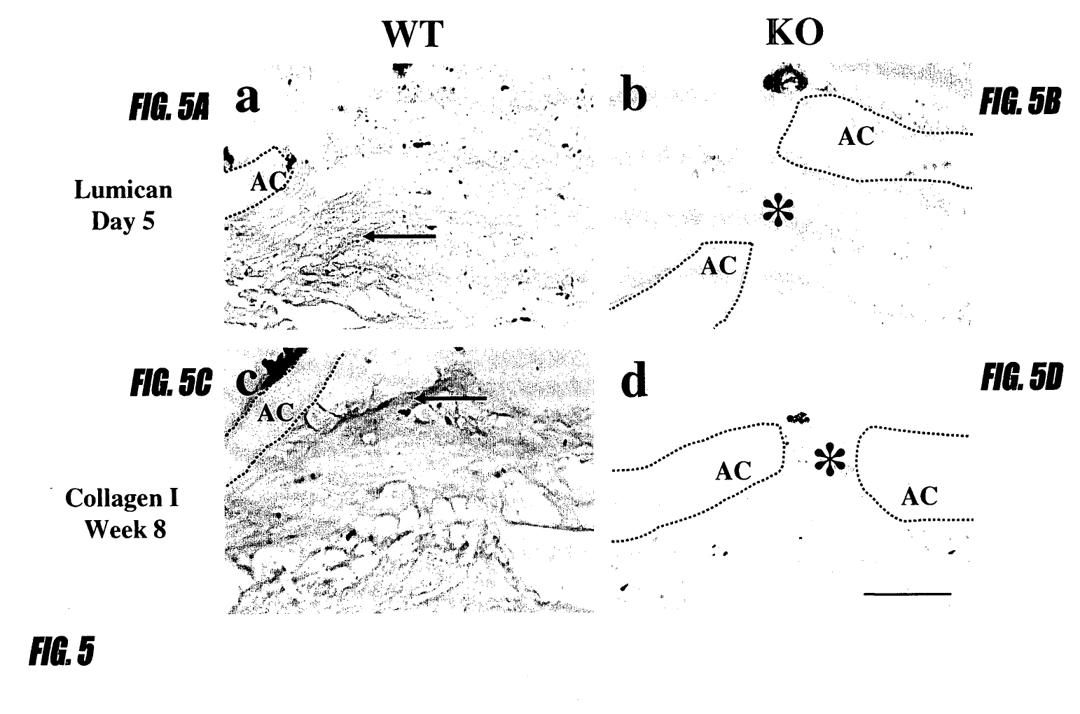

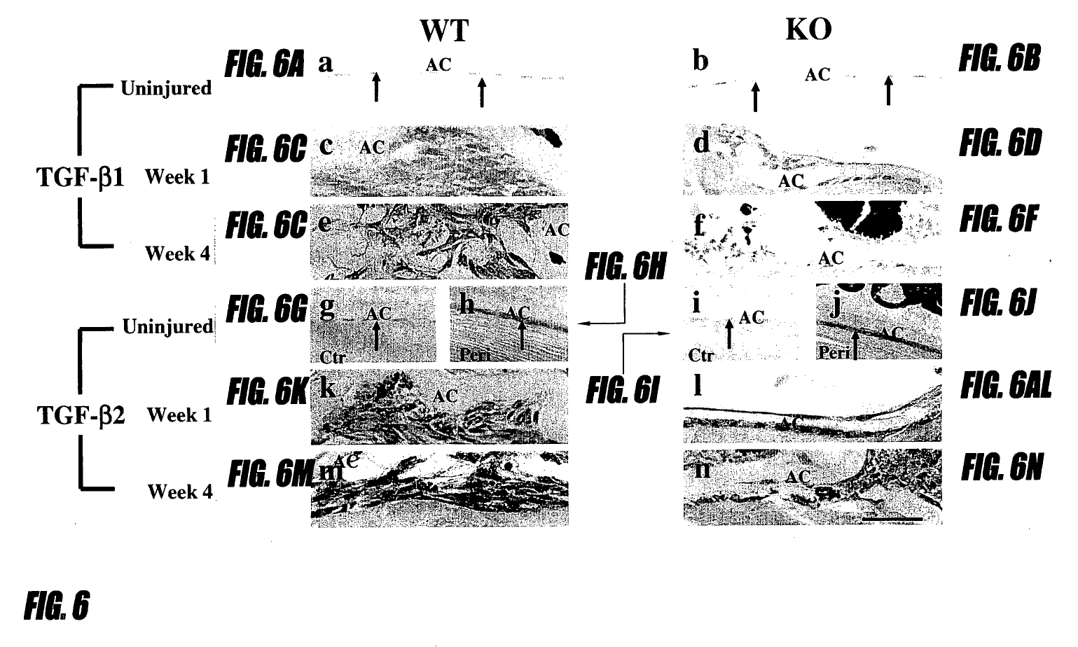

Uninjured lens epithelium was negative for αSMA protein and mRNA. WT lens epithelial cells were negative for αSma mRNA at day 1 post-injury, but first expressed it at day 3, whereas KO epithelial cells never expressed it throughout the interval up to week 8. Consistent with our previous observations that lens epithelial cells undergoing EMT in vivo start to express αSMA protein between days 3 and 5 after a lens capsular injury (Marcantonio, J. M. and Vrensen, G. F. 1999 Eye 13: 484-488; Hales, A. M. et al. 1994 Curr Eye Res 13: 885-890; Saika, S. et al. 1998 Exp Eye Res 66: 283-294; Saika, S. et al. 2003. Invest Ophthalmol Vis Sci 44: 2094-2102), immunohistochemical staining with anti-αSMA antibody showed that fibroblastic-like lens epithelial cells that populated the injury site in WT eyes were strongly labeled by 5 days post-injury (FIG. 4a) and continued to express αSMA at 1 and 2 weeks post-injury (FIG. 4c, e), returning to baseline at 8 weeks. Immunoreactivity for αSMA was strongest at 1 week post-injury. Injured eyes of Smad3 heterozygous mice showed a morphological EMT and expression of αSMA similar to that of WT mice, indicating that a single allele of Smad3 was sufficient to support EMT. In contrast, the more epithelial-like cells found in wounds in KO eyes were completely negative for αSMA and remained so even up to 8 weeks post-injury (FIG. 4b, d, f).

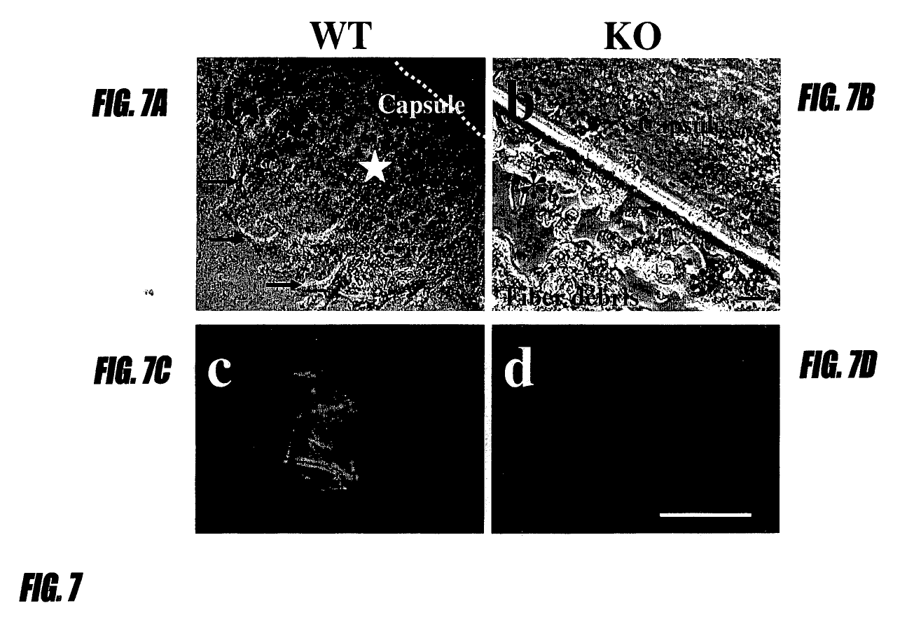

Expression of lumican, a small, leucine-rich keratan sulfate proteoglycan, precedes up-regulation of αSMA in lens cells during wound healing, since loss of lumican delayed the expression of αSMA in such cells post-injury (Saika, S. et al. 2003 Invest Ophthalmol Vis Sci 44: 2094-2102). To gain additional insight into the role of lumican in EMT of lens epithelium and to further identify the steps dependent on Smad3, we examined the pattern of lumican expression in KO lenses post-injury. Uninjured lens epithelial cells did not express lumican in either WT or KO mice. However, whereas WT lens epithelial cells at the capsular break began to express lumican protein as early as day 1 and cells expressing a fibroblastic morphology at 5 days post-injury were positive (FIG. 5a) and remained positive until 4 weeks post-injury, lens epithelial cells of KO mice were negative for lumican expression at all times examined (FIG. 5b).

As evidence that this block in EMT in lenses of KO eyes post-injury also prevented subsequent fibrosis, no staining for collagen types I (FIG. 5d), or V was evident in these eyes. In contrast, cells around the capsular break in WT eyes became weakly reactive to anti-collagen I antibody between days 2 and 3 post-injury and remained strongly reactive up to week 8 post injury (FIG. 5c). Together, these data indicate that Smad3 signaling is essential to injury-induced EMT of lens epithelial cells in vivo.

Late Induction of Expression of TGF-β1 Post-Injury Indicates a Role in Fibrosis but not EMT.

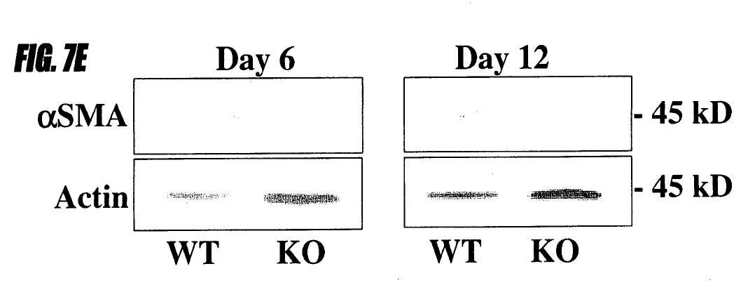

Although TGF-β2 predominates in the eye, (Jampel, H. D. et al. 1990 Curr Eye Res 9: 963-969; Connor, T. B. Jr. et al. 1989 J Clin Invest 83: 1661-1666) over-expression of TGF-β1 driven by the α-lens crystallin promoter results in EMT of the lens epithelium and formation of cataracts (Srinivasan, Y. et al. 1998 J Clin Invest 101: 625-634) and each of the three isoforms of TGF-β has been shown to be capable of inducing cataracterous changes in rat lenses in organ culture, albeit with different potencies (Gordon-Thomson C. et al. 1998 Invest Ophthalmol Vis Sci 39: 1399-1409). To address whether TGF-β1, rather than TGF-β2, might mediate EMT of lens epithelium post-injury in vivo, we used isoform-specific antibodies to assess their expression (Flanders, K. C. et al. 1991 Development 113: 183-191). Uninjured lens epithelial cells in WT and KO mice did not express detectable amounts of TGF-β1 (FIG. 6a, b). In WT mice, TGF-β1 was up-regulated in lens epithelial cells exhibiting a fibroblastic morphology at week 1 post-injury (FIG. 6c), increased in intensity until week 4 (FIG. 6e), and then returned to basal levels at week 8. Throughout the healing intervals examined, no up-regulation of TGF-β1 was observed in KO mice (FIG. 6d, f). These data are consistent with the observed lack of autoinduction of TGF-β1 in Smad3-null cells (Piek, E. et al. 2001 J Biol Chem 276: 19945-19953; Ashcroft, G. S. et al. 1999 Nat Cell Biol 1: 260-266), and with reduced levels of expression of TGF-β1 in skin of KO mice post-irradiation (Flanders, K. C. et al. 2002 Am J Pathol 160: 1057-1068). The late onset of TGF-β1 expression post-capsular injury and our observation that expression of αSMA protein was unperturbed at day 5 post capsular-injury in 2 week-old Tgf-β1-null mice indicate that TGF-β1 does not play a direct role in EMT, but that it might contribute to elaboration of ECM at later times post-injury. In contrast, there was no obvious difference in expression of TGF-β2 in eyes of WT and KO mice (FIG. 6g-n). TGF-β2 was expressed in peripheral lens epithelial cells in the proliferative zone, but not in central epithelia of uninjured lenses in both WT and KO mice (FIG. 6g-j). However, at week 1 post-injury, central epithelia around the capsular break became positive for expression of TGF-β2 (FIG. 6k, l), increasing by week 4 post-injury (FIG. 6m, n), and this diminished by week 8 post-injury in both WT and KO mice.

Outgrowth of a Migrating Epithelial Sheet and its Expression of αSMA is Perturbed by the Loss of Smad3.

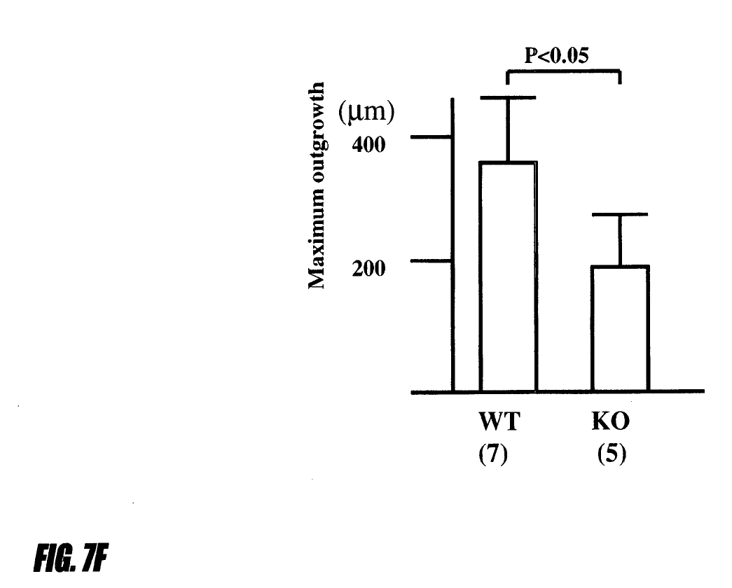

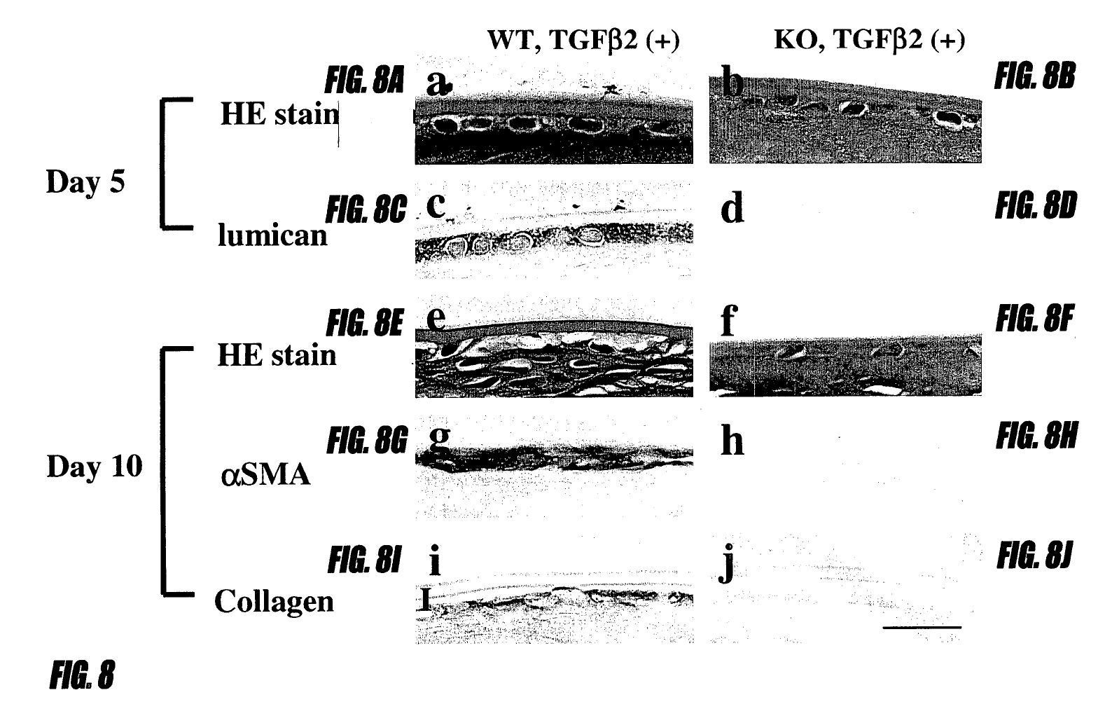

To further confirm that EMT of mouse lens epithelium requires Smad3 signaling, we examined EMT in explant cultures of lenses of WT and KO mice. WT lens epithelial cells exhibit a more robust outgrowth from the capsular specimens than do KO cells (FIG. 7a, b, f). Notably, WT cells located at the edge of the migrating epithelial sheet exhibited a fibroblast-like morphology and expressed αSMA (FIG. 7c), while no labeled cells were seen in cultures of KO specimens (FIG. 7d). This selective expression of αSMA by the WT lens epithelium was confirmed by western blotting at Day 6 and 12 of culture (FIG. 7e). Similar levels of αSMA protein were expressed by WT explant cultures at both timepoints examined, while the protein was undetectable at both timepoints in lysates of KO explant cultures.

TGF-β2-Mediated EMT in Lens Organ-Culture is Dependent on Smad3.

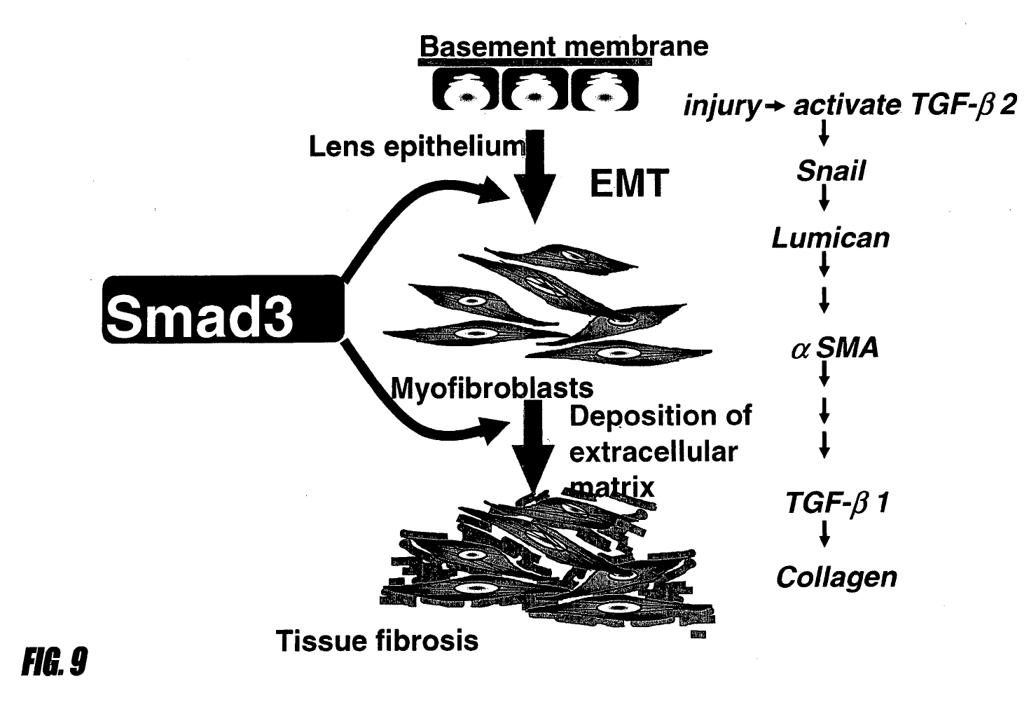

We have previously reported that organ-culture of mouse lens in the presence of 10 ng/ml of TGF-β2 for 10 days results in EMT and expression of αSMA by epithelial cells beneath the intact capsule (Saika, S. et al. 2003 Invest Ophthalmol Vis Sci 44:2094-2102). To confirm our in vivo results indicating that this process is dependent on Smad3, we cultured lenses from WT and KO eyes (FIG. 8) in the presence or absence of TGF-β2 (10 ng/ml) for periods up to 10 days. TGF-β2 up-regulated expression of lumican at day 5 in cultured lenses of WT but not KO (FIG. 8c, d). At this timepoint, there was still no evidence for morphological EMT or αSMA expression in cultures of either genotype (FIG. 8a, b), consistent with our previous finding that lumican expression precedes morphological evidence of EMT in lens epithelium (Saika, S. et al. 2003 Invest Ophthalmol Vis Sci 44:2094-2102). After 10 days culture in the presence of TGF-β2, lens epithelium of WT mice consisted of a multilayer of cells of a fibroblastic morphology (FIG. 8e) and expressed lumican, αSMA (FIG. 8g), and collagen type I (FIG. 8i), whereas the subcapsular cells of either KO lenses cultured in the presence of TGF-β2 (FIG. 8f, h, j) or WT lenses cultured in the absence of TGF-β2 retained an epithelial shape and failed to expressed markers of EMT.

Discussion

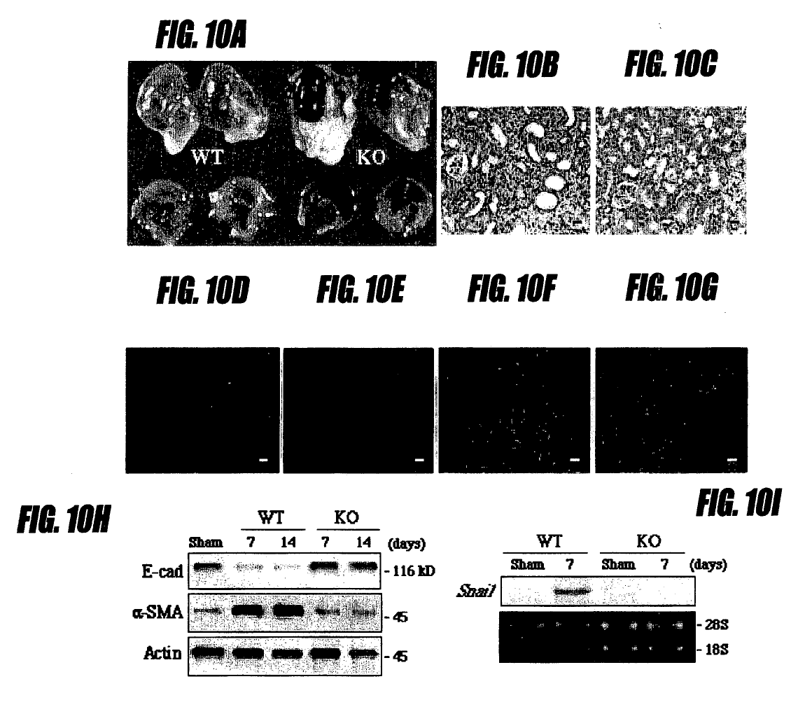

Our data demonstrate, for the first time, that EMT of lens epithelial cells post-anterior capsular injury in vivo is dependent on signaling through Smad3, a key signaling intermediate downstream of TGF-β and activin receptors. In the absence of Smad3, neither the earliest marker of EMT, snail, nor any of the other markers for later stages of EMT are expressed, including the proteoglycan lumican, αSMA, the hallmark of myofibroblasts, or collagen type I, a major component of the pathologic ECM (FIG. 9). While these data are consistent with previous data implicating TGF-β in EMT of lens epithelium (Hales, A. M. et al. 1994 Curr Eye Res 13: 885-890; Saika, S. et al. 2001 Exp Eye Res 72: 679-686; Saika, S. et al. 2002 Br J Ophthalmol 86: 1428-1433; Wormstone, I. M. et al. 2002 Invest Ophthalmol Vis Sci 43: 2301-2308), lens epithelial cells also express activin receptors (Obata, H. et al. 1999 Acta Ophthalmol Scand 77: 151-156), which could activate Smad3 signaling. However, based on 1) the finding that spontaneous EMT of primary porcine lens epithelial cells is also blocked by an anti-TGF-β antibody (FIG. 1), and 2) the ability of TGF-β2 to induce EMT in lens organ culture (FIG. 8), we propose that Smad3-dependent injury-induced EMT of lens epithelium is initiated by activation of TGF-β2 rather than by activin. Induction of TGF-β1 in mesenchymal-like cells at later times post-injury (FIG. 6) is consistent with a role of this isoform in elaboration of ECM, but not in induction of EMT of lens epithelium.

Others have shown that aqueous and vitreous contain inhibitors of TGF-β, such as α2-macroglobulin, which may afford protection from unwanted EMT and fibrogenesis, both basally and following injury (Schulz, M. W. et al. 1996 Invest Ophthalmol Vis Sci 37: 1509-1519). Additionally, it has been indicated that antibody therapies directed against TGF-β, such as CAT-152, may be able to prevent capsular opacification following cataract surgery (Wormstone, I. M. et al. 2002 Invest Ophthalmol Vis Sci 43: 2301-2308). The present studies demonstrating the central role of Smad3 in both EMT of lens epithelium and, by inference from previous studies, in the elaboration of collagens and other extracellular matrix proteins by cells expressing a mesenchymal phenotype (Verrecchia, F and Mauviel, A. 2002 J Invest Dermatol 118: 211-215; Ashcroft, G. S. et al. 1999 Nat Cell Biol 1: 260-266) (FIG. 9), now indicate that Smad3 should be another target for design of novel therapeutics.

EMT of cardiac endothelial cells is required for formation of the endocardial cushions in the atrioventricular canal of the developing heart (Romano, L. A. and Runyan, R. B. 2000 Dev Biol 223: 91-102; Camenisch, T. D. et al. 2002 Dev Biol 248: 170-181). Unlike the EMT of lens epithelium described here, this TGF-β2-dependent EMT is independent of Smad3, since heart development is normal in Smad3 null mice (Yang, X. et al. 1999 EMBO J. 18: 1280-1291). Rather EMT of cardiac endothelial cells is dependent on expression of the type III receptor (TβRIII) and expression of slug, which, like snail, represses expression of E-cadherin (Romano, L. A. and Runyan, R. B. 2000 Dev Biol 223: 91-102; Brown, C. B. et al. 1999 Science 283: 2080-2082). Similarly, TGF-β3-dependent EMT of medial edge epithelial cells, critical in fusion of the palatal shelves later in development, also occurs independently of Smad3 and correlates with expression of TβRIII and phosphorylation of Smad2 (Cui, X. M. and Schuler, C. F. 2000 Int J Dev Biol 44: 397-402; Cui, X. M. et. al. 2003 Dev Dyn 227:387-394).

Several studies of EMT of epithelial cell lines in culture have also indicated that the process is independent of Smad3 and that other pathways including phosphatidylinositol 3-kinase, RhoA, and MAPK pathways are involved (Bakin, A. V. et al. 2000 J Biol Chem 275:36803-36810; Bhowmick, N. A. et al. 2001 Mol Biol Cell 12:27-36; Janda, E. et al. 2002 J Cell Biol 156:299-314; Oft, M. et al. 1996 Genes Dev 10:2462-2477; Bhowmick, N. A. et al. 2001 J Biol Chem 276:46707-46713). However, recent studies utilizing a mutant TβRI unable to bind or activate Smad2/3 but still competent to signal through MAPK pathways, clearly show that Smad activation is also required in certain cells, indicating that the Smad pathway is necessary but possibly not sufficient to effect EMT of these cell lines driven by TGF-β in vitro (Itoh, S. et al. 2003 J Biol Chem 278:3751-3761; Yu, L. et al. 2002 EMBO J. 21:3749-3759). Based on our demonstration that EMT of lens epithelium in vivo is blocked in the absence of Smad3, we hypothesize that signaling through this pathway is required for the early stages of the injury-dependent multi-stage transition of a lens epithelial cell to a mesenchymal phenotype, but possibly no longer necessary in a subset of established cell lines which may already have transited initial Smad3-dependent steps required for induction of EMT (Bakin, A. V. et al. 2000 J Biol Chem 275:36803-36810; Bhowmick, N. A. et al. 2001 Mol Biol Cell 12:27-36; Bhowmick, N. A. et al. 2001 J Biol Chem 276:46707-46713). Supporting this argument, outgrowths of lens epithelial cells from KO mice do not express αSMA, a marker of EMT, even though it has been shown that Smad3 is not essential for induction of αSMA by TGF-β in cultured dermal fibroblasts. Similarly, in cultured hepatic stellate cells, an inhibitor of the Smad pathway, Smad7, blocks the formation of cytoskeletal fibers immunoreactive for αSMA, without changing the level of αSMA protein, indicating that TGF-β/Smad signaling is required for the assembly of αSMA in the cytoskeleton, but not for its synthesis (Dooley, S. et al. 2003 Gastroenterology 125:178-191). Taken together, these data support our arguments that TGF-β/Smad3 signaling is required at a very early point in the process of EMT, before the step in which αSMA is induced.

Although the entire sequence of molecular events involved in Smad3-dependent EMT of lens epithelial cells is still not known, we have been able to characterize some of the early steps in the process. Our data show that snail, a zinc finger transcription factor that has been strongly linked to EMT (Carver, E. A. et al. 2001 Mol Cell Biol 21:8184-8188; Nieto, M. A. et al. 2002 Nat Rev Mol Cell Biol 3:155-166), is up-regulated in α-TN4, an SV40-transformed mouse lens epithelial cell line, as early as 30 min after TGF-β addition, and prior to up-regulation of αSMA. While our present data cannot address a putative requirement for snail for EMT, we do show that it is expressed in lens epithelial cells at the edge of the capsular break 1 day after injury, prior to the expression of any other markers of EMT. KO lens epithelia neither underwent EMT nor expressed snail, consistent with the finding that snail is a Smad3-dependent immediate-early gene target of TGF-β (Yang, Y. C. et al. 2003 PNAS USA 100:10269-10274). Ectopic expression of snail in epithelial cell lines is sufficient to induce EMT and expression of mesenchymal markers (Cano, A. et al. 2000 Nat Cell Biol 2:76-83), indicating a model in which it acts as a master switch controlling the subsequent transcriptional changes. While the snail homologue, slug, was basally expressed in α-TN4 cells, whether it, or SIP1, another TGF-β-inducible, Smad-interacting zinc-finger protein involved in transcriptional suppression of E-cadherin expression, might also be involved in EMT of lens epithelium in vivo is not known at the present time (Nieto, M. A. et al. 2002 Nat Rev Mol Cell Biol 3:155-166; Comijn, J. et al. 2001 Mol Cell 7:1267-1278) but is now envisioned.

Previous studies have shown that many ECM molecules, in addition to performing structural roles, can also facilitate the conversion of cells to αSMA-positive myofibroblasts under pathological conditions (Boukamp, P. and Fusenig, N. E. 1993 J Cell Biol 120:981-993; Serini, G. and Gabbiani, G. 1999 Exp Cell Res 250:273-283; Zeisberg, M. et al. 2001 Am J Pathol 159:1313-1321). For example, in cultured fibroblasts, fibronectin EIIIA enhances and vitronectin suppresses αSMA expression (Scaffidi, A. K. et al. 2001 J Cell Sci 114:3507-3516; Serini, G. et al. 1998 J Cell Biol 142:873-881). The requirement of β1 integrin expression for EMT in NMuMg cells (Bhowmick, N. A. et al. 2001 J Biol Chem 276:46707-46713) and lens epithelial cells (Zuk, A. and Hay, E. D. 1994 Dev Dyn 201:378-393) again underscores the importance of ECM signaling in EMT. We have recently reported that lumican, a core protein of keratan sulfate proteoglycan (Saika, S. et al. 2000 J Biol Chem 275:2607-2612), is transiently expressed in healing lens epithelial cells following a puncture injury and that loss of lumican by gene targeting results in a significant delay in EMT of lens epithelial cells (Saika, S. et al. 2003 Invest Ophthalmol Vis Sci 44:2094-2102). In the present study, we show that loss of Smad3 blocks the injury-related induction of lumican expression in lens epithelial cells. While this loss of lumican expression may contribute secondarily to the block of EMT in the Smad3-null lens epithelium, it is unlikely to be a primary target since its loss results in a delay, but not a block of EMT (Saika, S. et al. 2003 Invest Ophthalmol Vis Sci 44:2094-2102).

We have previously proposed that an actual inhibitor of the Smad3 pathway would promote more rapid closure of cutaneous wounds (Ashcroft, G. S. et al. 1999 Nat Cell Biol 1:260-266). However, the wounding response in skin involves many different cell types including keratinocytes, fibroblasts, and inflammatory cells, each of which is affected in cell-specific ways by modulation of this signaling pathway. Thus the decreased expression of collagen and certain other matrix proteins in Smad3 null wounds may be not be desirable in certain situations where wound strength might be critical. However, in the crystalline lens, the lens epithelial cell is the predominant cell lineage. Since lens epithelium is known to undergo pathologic EMT following traumatic injury, as in cataract surgery and implantation of an artificial lens (Hales, A. M. et al. 1994 Curr Eye Res 13:885-890; Saika, S. et al. 2001 Exp Eye Res 72:679-686; Saika, S. et al. 2002 Br J Ophthalmol 86:1428-1433; Wormstone, I. M. et al. 2002 Invest Ophthalmol Vis Sci 43:2301-2308), and since this EMT can lead to production of ECM and to opacification and contraction of the capsule containing the artificial lens (Marcantonio, J. M. & Vrensen, G. F. 1999 Eye 13:484-488), there is envisioned to be therapeutic benefit to inhibition of EMT, as proposed for antibodies to TGF-β2 (Wormstone, I. M. et al. 2002 Invest Ophthalmol Vis Sci 43:2301-2308).

Our findings implicating Smad3 signaling in EMT of lens epithelium may have broader significance based on our preliminary results using a model of retinal detachment, which show that Smad3 is also required for EMT of retinal pigment epithelium in mice. In proliferative vitreoretinopathy, which is the most common cause of failure in retinal reattachment surgery, EMT of retinal pigment epithelial cells can lead to fibrosis and to traction detachment of the retina (Connor, T. B. Jr. et al. 1989 J Clin Invest 83:1661-1666). Together, these results indicate that suppression of EMT in ocular cells by interfering with Smad3 signaling should have clinical application in treatment of these and other eye disorders.

### EXAMPLE 2

### Targeted Distribution of TGF-β1/Smad3 Signaling Protects Against Renal Tubulointerstitial Fibrosis Induced by Unilateral Ureteral Obstruction

Tubulointerstitial fibrosis is the final common result of a variety of progressive injuries leading to chronic renal failure. Transforming growth factor-β (TGF-β) is reportedly upregulated in response to injurious stimuli such as unilateral ureteral obstruction (UUO), causing renal fibrosis associated with epithelial-mesenchymal transition (EMT) of the renal tubules and synthesis of extracellular matrix. We now show that mice lacking Smad3 (Smad3^{ex8/ex8}), a key signaling intermediate downstream of the TGF-β receptors, are protected against tubulointerstitital fibrosis following UUO as evidenced by blocking of EMT and abrogation of monocyte influx and collagen accumulation. Culture of primary renal tubular epithelial cells from wild-type or Smad3-null mice confirms that the Smad3 pathway is essential for TGF-β1-induced EMT and autoinduction of TGF-β1. Moreover, mechanical stretch of the cultured epithelial cells mimicking renal tubular distention due to accumulation of urine after UUO in vivo induces EMT following Smad3-mediated upregulation of TGF-β1. Exogenous bone-marrow monocytes accelerate EMT of the cultured epithelial cells and the renal tubules in the obstructed kidney after UUO via Smad3 signaling. Together the data demonstrate that the Smad3 pathway is central to the pathogenesis of interstitial fibrosis and indicate that inhibitors of this pathway should have clinical application in treatment of obstructive nephropathy.

Introduction

Renal interstitial fibrosis is a progressive and potentially lethal disease caused by diverse clinical entities including urinary tract obstruction, chronic inflammation and diabetes (Eddy, A. A. 1996 J Am Soc Nephrol 7:2495-508; Remuzzi, G., and Bertani, T. 1998 N Eng J Med 339:1448-1456; Stahl, P. J., & Felsen, D. 2001 Am J Pathol 159:1187-1192). TGF-β plays a pivotal role in chronic inflammatory changes of the interstitium and accumulation of extracellular matrix during renal fibrogenesis (Blobe, G. C. et al. 2000 N Eng J Med 342:1350-1358; Border, W. A., and Noble, N. A. 1997 Kidney Int 51:1388-1396). Emerging evidence indicates that TGF-β initiates the transition of renal tubular epithelial cells to myofibroblasts, the cellular source for extracellular matrix deposition, leading ultimately to an irreversible renal failure (Yang, J., & Liu, Y. 2001 Am J Pathol 159:1465-1475; Zeisberg, M. et al. 2002 Am J Pathol 160:2001-2008; Iwano, M. et al. 2002 J Clin Invest 100:341-350; Li, J. H. et al. 2002 J Am Soc Nephrol 13:1464-1472). To understand the mechanisms underlying the pathogenesis of renal fibrotic disorders, it is therefore essential to identify the molecular events involved in induction of epithelial-mesenchymal transition in this disease process.

In the past few years, the receptors and signal transduction pathways mediating effects of TGF-β on cells have been identified, now enabling identification of the specific pathways involved in pathogenetic events dependent on this cytokine. TGF-β type I and type II transmembrane receptor serine/threonine kinases transduce downstream signals via novel cytoplasmic latent transcription factors called Smad proteins. Smad2 and Smad3 are phosphorylated directly by the type I receptor kinase after which they partner with Smad4 and translocate to the nucleus where they act as transcriptional regulators of target genes, including those essential for apoptosis, differentiation and growth inhibition (Massagué, J., and Wotton, D. 2000 EMBO J. 19:1745-1754; ten Dijke, P. et al. 2002 J Cell Physiol 191:1-16; Derynck, R. et al. 1998 Cell 95:737-740). Unlike the targeted deletion of Smad2 which results in embryonic lethality, deletion of Smad3 results primarily in impaired mucosal immunity in mice, shortening their life span to 1-6 months (Yang, X. et al. 1999 EMBO J. 18:1280-1291). We have now utilized these mice (Smad3^{ex8/ex8}) and their wild-type littermate controls to study the role of the Smad3 signaling pathway in the pathogenesis of fibrosis induced by unilateral ureteral obstruction (UUO), a model for renal tubulointerstitial fibrosis and obstructive nephropathy (Klahr, S., & Morrissey, J. 2002 Am J Physiol Renal Physiol 283:F861-F875).

Materials and Methods

Unilateral Ureteral Obstruction.

Smad3-null (Smad3^{ex8/ex8}) mice, 6 to 8-week-old, 20 to 30 g were generated as described (Yang, X. et al. 1999 EMBO J. 18:1280-1291). Under general anesthesia, the right proximal ureter was exposed and double-ligated after a right back incision. All the experimental procedures were approved by Animal Care and Use Committee of Wakayama Medical University, Wakayama.

Primary Culture of Renal Tubular Epithelial Cells.

Minced kidneys were washed with 3 changes of cold PBS containing 1 mM EDTA and digested in 0.25% trypsin solution (Gibco BRL, Grand Island, N.Y.) in a shaking incubator at 37° C. for 2 h. Trypsin was neutralized with growth medium (DMEM/10% FBS containing 100 unit/ml penicillin and 0.1 mg/ml streptomycin). The suspension was triturated by pipetting and passed through a 100 μm cell strainer (Becton Dickinson Labware, Franklin Lakes, N.J.). The filtrate consisting mostly of dispersed renal tubules was plated onto culture dishes (Nalge Nunc International, Naperville, Ill.) and 2-well chamber slides (Nunc Lab-Tek II-CC2, Nalge Nunc International), and incubated at 37° C. in a CO₂ incubator with medium changes every 2 days. Experiments were carried out in serum-free DMEM. EMT was induced by an addition of 10 ng/ml TGF-β1 (R&D Systems, Minneapolis, Minn.). Mouse monoclonal anti-TGF-β neutralizing antibody (clone: 1D11, R & D Systems) was used at a concentration of 20 μg/ml with mouse IgG (Sigma, St Louis, Mo.) as a control.

Mechanical Stretching.

Cells grown on culture plates with flexible bottoms coated with type I collagen (BioFlex, Flexcell International Corp., Mckeesport, Pa.) were subjected to a mechanical strain of downward deformation by a computer-controlled vacuum using a Flexercell FX-2000 at an alternate cycles of 5 seconds stretch and 5 seconds relaxation, 15% elongation rate in a CO₂ incubator at 37° C.

Bone-Marrow Monocytes.

Mononuclear cells in the bone marrow were collected from tibias and femurs of 7-week-old mice and cultivated for 7 days in growth medium containing 10 ng/ml of recombinant mouse macrophage-colony stimulating factor (R & D Systems) as described (Feldman, G. M. et al. 1997 Blood. 90:1768-1776). Monocytes (5×10⁴) suspended in 50 μl of DMEM were plated into primary culture of renal tubular epithelial cells in a 2-well chamber slide preconditioned with 1 ml of serum-free DMEM for 24 h. Co-culture was continued for 48. For transplantation of monocytes, the right kidney and proximal ureter were exposed after a right back incision under general anesthesia. Monocytes (2.5×10⁵) suspended in 20 μl of DMEM were injected into the renal subcapsular space using a Hamilton syringe with 26-gauge needle and then the ureter was double-ligated. Mice were sacrificed at day 3 after operation.

Histology and Immunofluorescence.

Histological sections were prepared from tissues fixed in 4% paraformaldehyde in 0.1 M phosphate buffer, pH 7.4, and embedded in paraffin. Cryosections and bottom sheets of BioFlex were fixed in cold acetone and subjected to indirect immunofluorescence with anti-E-cadherin (clone: DECMA-1, Sigma, St Louis, Mo.), anti-α-SMA (clone: 1A4, NeoMarkers, Fremont, Calif.), anti-mouse type I collagen (Southern Biotechnology, Birmingham, Ala.) and anti-mouse F4/80 antibodies (clone: A3-1, BMA, Augst, Switzerland). As second antibodies, FITC-anti-rat IgG (Sigma), TRITC-anti-mouse IgG (Sigma) or Cy3-anti-goat IgG (Sigma) were used.

Immunoblot.

Cells and tissues were lysed in buffer containing 1% Nonidet P-40, 25 mM Tris-HCl, pH 7.5, 150 mM NaCl, 10 mM EDTA and 1:50 dilution of a protease inhibitor cocktail (P-2714, Sigma). Proteins were separated by SDS-PAGE, transferred to nitrocellulose membranes, and blocked in 5% skim milk in PBS. After incubation with primary antibodies against E-cadherin (clone: 36, Transduction Laboratories, Lexington, Ky.), α-SMA (clone: 1A4) and actin antibodies (Sc-1616, Santa Cruz Biochemicals, Santa Cruz, Calif.), blots were reacted with POD-conjugated goat anti-mouse IgG secondary antibody (Sigma) and developed with ECL (Amersham Biosciences, Buckingshire, UK).

In Situ Hybridization.

Digoxigenin-11-UTP-labelled antisense riboprobes were prepared with an RNA-labeling kit (Roche Diagnostics Corp.-Boeringer Mannheim, Indianapolis) for in situ hybridization as described (Gong, Y. et al. 2001 Cell. 107:513-523). The mouse α-SMA, Snail and TGF-β1 RNA probes were transcribed from PCR products using following primers: α-SMA, 5′-CTGCTCTGCCTCTAGCACAC-3′ (SEQ ID NO: 5) and 5′-TTAAGGGTAGCACATGTCTG-3′ (SEQ ID NO: 6); Snail, 5′-ACACTGGTGAGAAGCCATTC-3′ (SEQ ID NO: 7) and 5′-AGTTCTATGGCTCGAAGCAG-3′ (SEQ ID NO: 8); TGF-β1, 5′-CACGTGGAAATCAACGGGAT-3′ (SEQ ID NO: 9) and 5′-GCGCACAATCATGTTGGACA-3′ (SEQ ID NO: 10) from complete mouse mRNA. Sections were subjected to a Ventana HX system (Ventana Medical Systems, Inc., Tucson, Ariz.) according to the manufacturer's instruction. After hybridization, sections were washed 3 times in 0.1% SCC high stringency solution at 65° C. and incubated with alkaline phosphatase-conjugated anti-digoxigenin Fab fragments (Roche). The color was developed in freshly prepared substrate solution NBT-BCIP (Digoxigenin detection kit, Roche).

Northern Blot.

Total RNA was extracted with TRIzol reagent (Invitrogen, Carlsbad, Calif.). RNA (20 μg/lane), separated in 1% agarose-formaldehyde gels and transferred to Hybond-HX nylon membranes (Amersham). Membranes were hybridized with cDNA probes for mouse Snail and TGF-β1 mRNA labeled with [P³²] dCTP by random primed DNA synthesis using the same primers as above (Rediprime II, Amersham Pharmacia Inc., Piscataway, N.J.). Filters were exposed to X-ray film at −80° C. for 2 to 3 d. Band intensities were normalized to those 28S and 18S ribosomal bands of ethidium bromide staining.

Immunoassay of TGF-β1.

Protein extracts from kidneys (Yang, J., and Liu, Y. 2001 Am J Pathol 159:1465-1475) and cell culture medium were used for determination of active TGF-β1 with a Quantikine TGF-β1 assay kit (R & D Systems). Samples were acidified for total TGF-β1 assay. Values were expressed as pg/mg protein for the protein extract or pg/cell number for cell culture medium.

Hydroxyproline Assay.

Tissue samples were hydrolysed in 6 N HCl for 12 h at 110° C. (50 mg/ml). Hydroxyproline content of supernatant solution was assayed by the method as described (Kivirikko, K. I. et al. 1967 Anal Biochem 19:249-255). Values were expressed as μg/mg tissue.

Statistics.

The results were expressed as the mean±standard deviation. Student's unpaired t test and an analysis of multiple variance by Scheffe's method were used for statistical comparison. A P value less than 0.05 was considered to indicate statistical significance.

Results

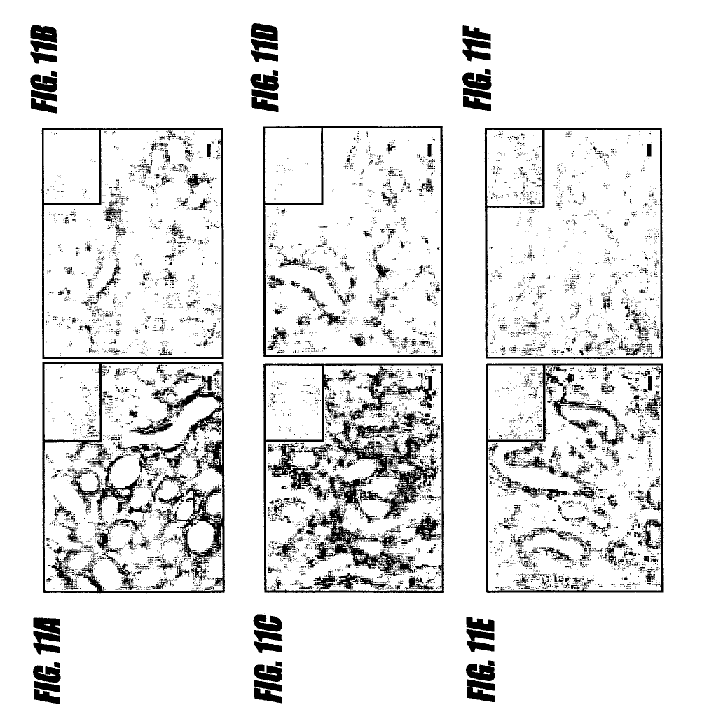

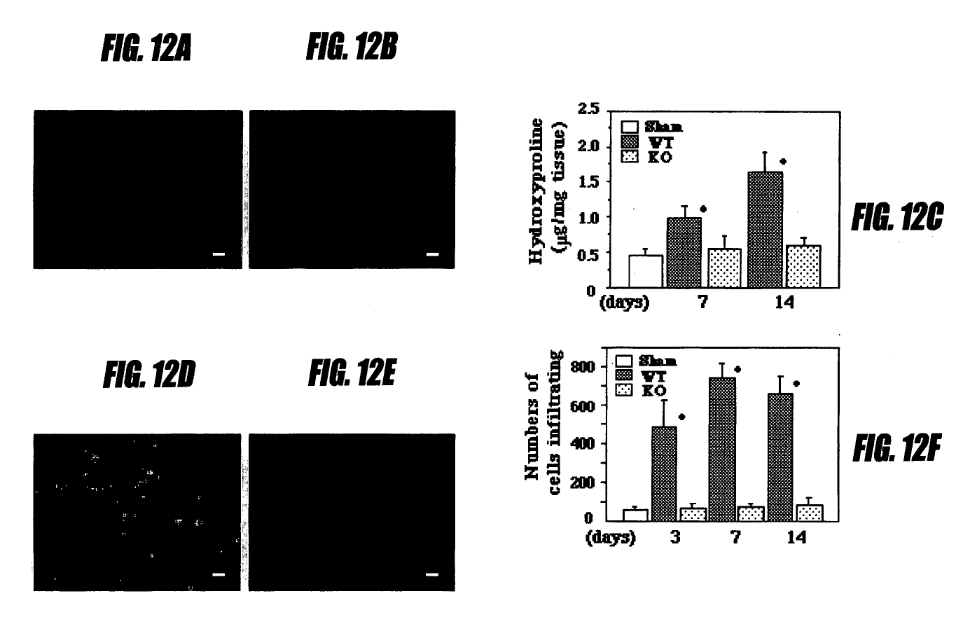

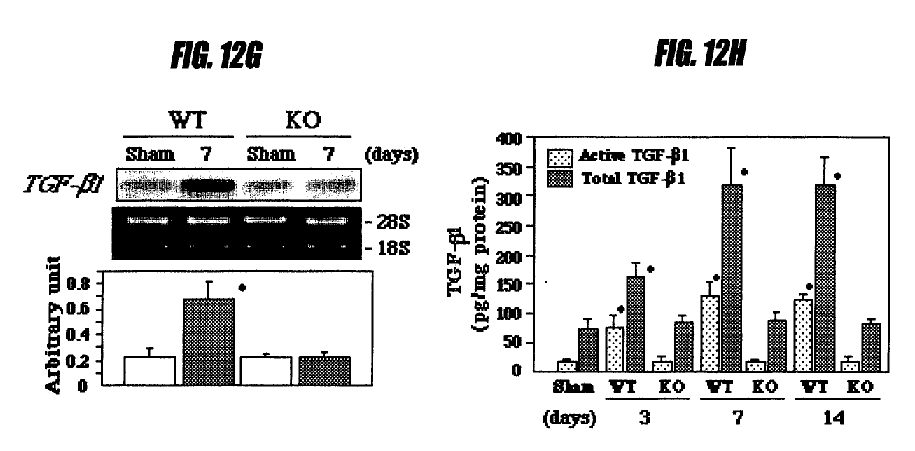

Renal architecture is preserved after unilateral ureteral obstruction in mice lacking Smad3. Two weeks after UUO, obstructed kidneys of wild-type mice were enlarged and exhibited dilated pelves and calyces, and a thin rim of remaining cortex, while an appreciable amount of the renal parenchyma was preserved in kidneys of Smad3-null littermates (FIG. 10a). Obstructed kidneys of wild-type mice showed fibrotic changes with dilated renal tubules accompanied by proliferation of fibroblastic cells and influx of inflammatory mononuclear cells (FIG. 10b), while the normal architecture was preserved in obstructed kidneys of Smad3-null mice (FIG. 10c). Dual immunofluorescence showed a marked reduction of E-cadherin staining with concomitant expression of α-smooth muscle actin (α-SMA) in kidneys of wild-type mice at days 7 and 14 after UUO (FIG. 10d, e). At both of these timepoints, renal tubules of Smad3-null mice remained positive for E-cadherin and α-SMA was restricted to vascular smooth muscle cells (FIG. 10f, g). Immunoblotting also showed a marked reduction of E-cadherin concomitant with an increase in α-SMA in wild-type mice, in the absence of any differences in sham-operated wild-type mice and Smad3-null mutants with or without UUO (FIG. 10h). Snail, a potent repressor of transcription of the E-cadherin gene (Nieto, M. A. 2002 Nat Rev Mol Cell Biol 3:155-166; Cano, A. et al. 2000 Nature Cell Biol 2:76-82), was expressed in kidneys of wild-type mice but not Smad3-null littermates at day 7 after UUO (FIG. 10i). In situ hybridization showed that Snail mRNA was specifically localized to renal tubular epithelial cells of wild-type mice 7 days after UUO (FIG. 11a), whereas α-SMA mRNA was detected in both renal tubular epithelial cells and fibroblastic cells adjacent to the renal tubules (FIG. 11c). No hybridization to either the Snail or α-SMA mRNA probe was detected in obstructed kidneys from Smad3-null mice (FIG. 11b, d). These findings indicate that the transition of renal tubular epithelial cells to myofibroblasts is dependent on a Smad3-specific mechanism.

Renal fibrosis induced by unilateral ureteral obstruction is prevented in Smad3 null mice. Two weeks after UUO, more type I collagen was deposited in obstructed kidneys of wild-type as compared with Smad3-null mice (FIG. 12a, b). Hydroxyproline content, a measure of total collagen, increased 2 to 3.5-fold in the obstructed kidneys of wild-type mice whereas no changes were seen in kidneys of either Smad3-null littermates or sham-operated wild-type mice (FIG. 12c). A greater numbers of monocytes as identified by the immunofluorescence of F4/80 antigen infiltrated into the interstitium of obstructed kidneys in wild-type as compared with Smad3-null mice (FIG. 12d, e). Numbers of monocytes per unit area in obstructed kidneys of wild-type mice increased 6 to 10-fold after UUO for 3, 7 and 14 days while no changes were seen in Smad3-null littermates or sham-operated wild-type mice (FIG. 12f). Northern blot analysis of TGF-β1 mRNA also showed higher expression in obstructed kidneys of wild-type mice than that of sham-operated counterparts, or Smad3-null mice with or without UUO (FIG. 12g). In situ hybridization of TGF-β1 mRNA was enhanced in renal tubules and mononuclear cells, consisting mostly of monocytes, infiltrating the interstitium of the obstructed kidneys in wild-type mice compared to Smad3-null counterparts (FIG. 11e, f). The concentrations of active and total TGF-β1 in extracts of obstructed kidneys of wild-type mice were 3 to 6-fold and 2 to 4-fold higher than those of Smad3-null or sham-operated wild-type mice after UUO for 3, 7 and 14 days, respectively (FIG. 12h). Together, these results show that none of the classical hallmarks of obstructive kidney disease seen in wild-type mice are found in mice lacking Smad3, indicating that this pathway is essential in transducing the effects of the ureter blockage.

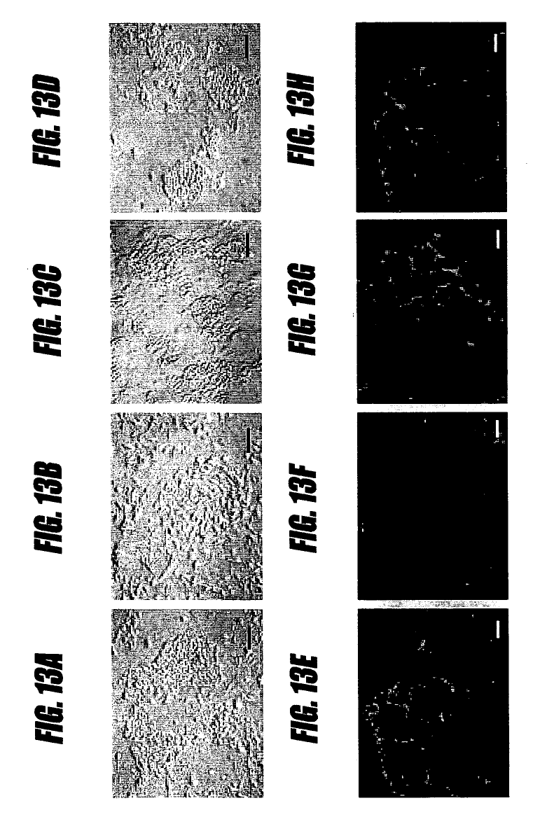

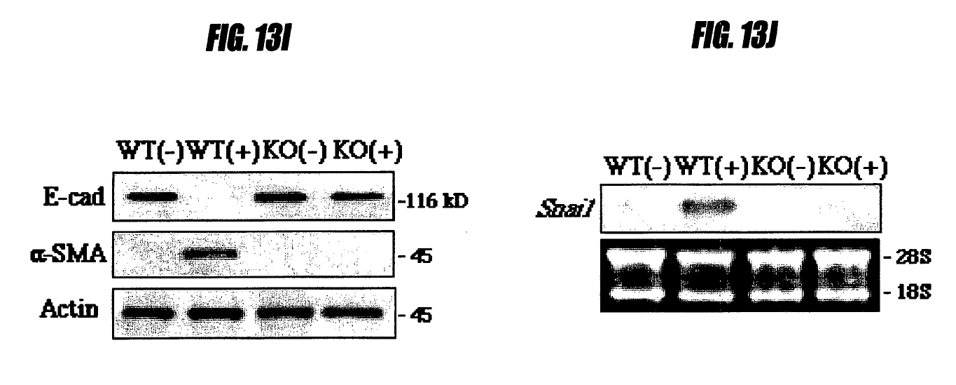

Epithelial-mesenchymal transition requires TGF-β1/Smad3 signaling. To ascertain whether these effects of the Smad3 pathway could be mediated by TGF-β, primary renal tubular epithelial cells were cultured from wild-type and Smad3-null mice. Experiments were conducted 5 to 7 days later when greater than 95% of cells were E-cadherin positive in regions of cell-cell adhesion. Treatment of wild-type epithelial cells with exogenous TGF-β1 resulted in a phenotypic change from cells exhibiting an epithelial-like cobblestone appearance to cells with a spindle-shaped, fibroblastic appearance (FIG. 13a, b), while TGF-β1-treated Smad3-null cells retained features of an epithelial monolayer (FIG. 13c, d). Marked reduction of E-cadherin and de novo expression of α-SMA were demonstrated by dual immunofluorescence in wild-type cells treated with TGF-β1 (FIG. 13f). These changes were not seen in untreated wild-type cells and Smad3-null cells with or without TGF-β1 treatment (FIG. 13e, g, h). Immunoblot analyses of E-cadherin and α-SMA in cell lysates from wild-type and Smad3-null epithelial cells in the absence or presence of TGF-β1 confirmed these findings (FIG. 13i). Treatment of wild-type epithelial cells, but not Smad3-null cells, with TGF-β1 also resulted in de novo expression of Snail mRNA (FIG. 13j), consistent with the data obtained in the in vivo model (FIG. 11a, b). These results indicate that the Smad3 pathway is essential for TGF-β1-induced EMT in the primary culture of renal tubular epithelial cells.

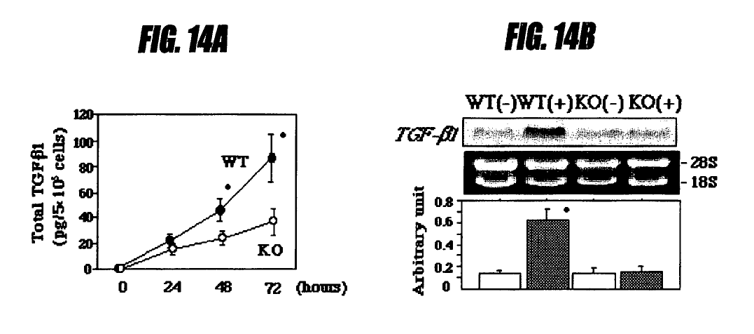

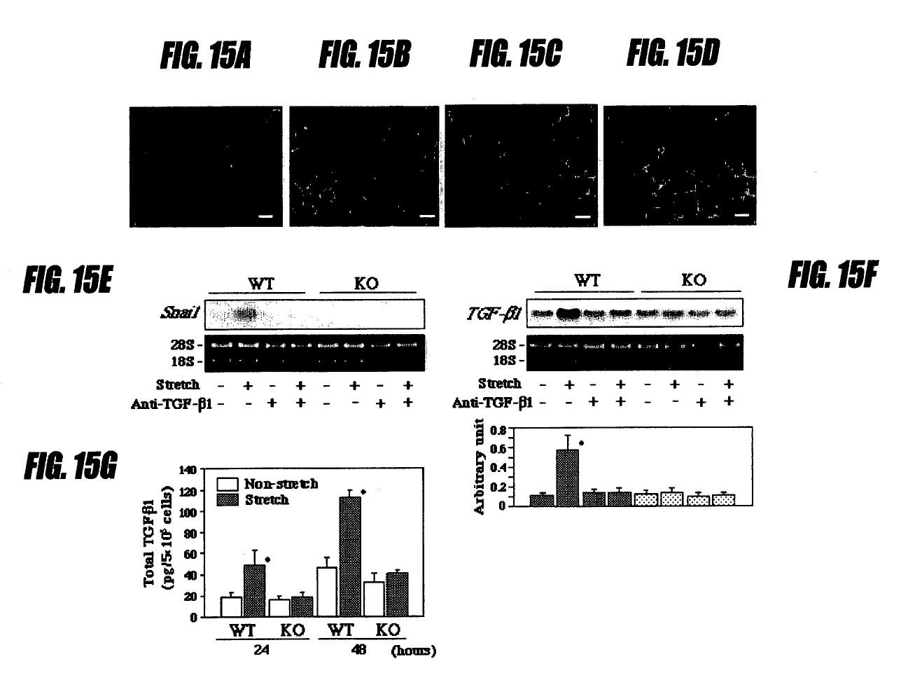

Autoinduction of TGF-β1 in primary culture of renal tubular epithelial cells. The concentration of total TGF-β1 in the culture medium of renal tubular epithelial cells increased time-dependently up to 72 h, with levels being significantly higher in medium of wild-type as compared with Smad3-null cells (FIG. 14a). To investigate whether these elevated levels of TGF-β1 could result from self-amplifying autocrine effects, TGF-β1 mRNA expression was determined in the presence or absence of exogenous TGF-β1. Addition of TGF-β1 to wild-type, but not to Smad3-null, epithelial cells enhanced expression of TGF-β1 mRNA compared with non-treated wild-type control cells (FIG. 14b), indicating that Smad3 signaling was essential to the autoinduction.

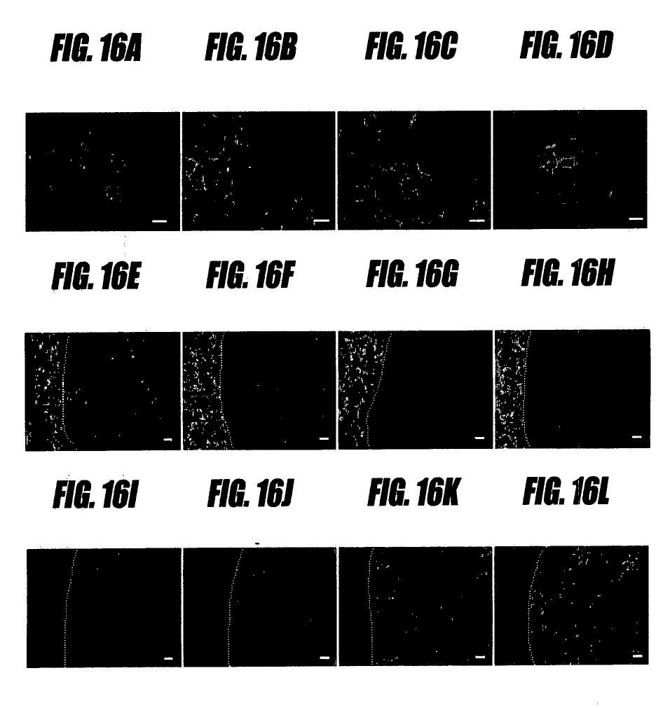

Stretch-induced upregulation of TGF-β1 and epithelial-mesenchymal transition. An in vitro experimental model of mechanically stretched renal tubular epithelial cell culture was used to model the pathogenetic effects of renal tubular distention by urine in UUO (Miyajima, A. et al. 2000 J Urol 164:1729-1734). Cyclic stretching of cultured wild-type cells elicited EMT as characterized by their phenotypic transition to αSMA-expressing myofibroblasts with marked reduction of cell membrane-localized E-cadherin (FIG. 15a). This stretch-induced EMT was abolished by a neutralizing antibody against TGF-β1 (FIG. 15b). No phenotypic conversion was found in Smad3-null cells under any conditions (FIG. 15c, d). Mechanical stretch also induced de novo expression of Snail mRNA only in wild-type cells, and this was blocked by treatment with a neutralizing anti-TGF-β1 antibody, coincident with effects on EMT (FIG. 15e). Increased expression of TGF-β1 mRNA induced by mechanical stretching was also restricted to wild-type cells and this was reversed by the treatment with a neutralizing anti-TGF-β1 antibody (FIG. 15f). Total TGF-β1 concentration in the culture medium was elevated more than 2-fold in stretched wild-type cells compared with non-stretched control cultures after either 24 or 48 h of mechanical stretching. No significant changes were observed in similarly treated cultures of Smad3-null cells (FIG. 15g). These results clearly show that production of TGF-β1 in this model is Smad3 dependent and further, that the TGF-β produced by renal epithelial cells in response to mechanical injury in vitro and, by implication, in response to UUO in vivo, is required for EMT.

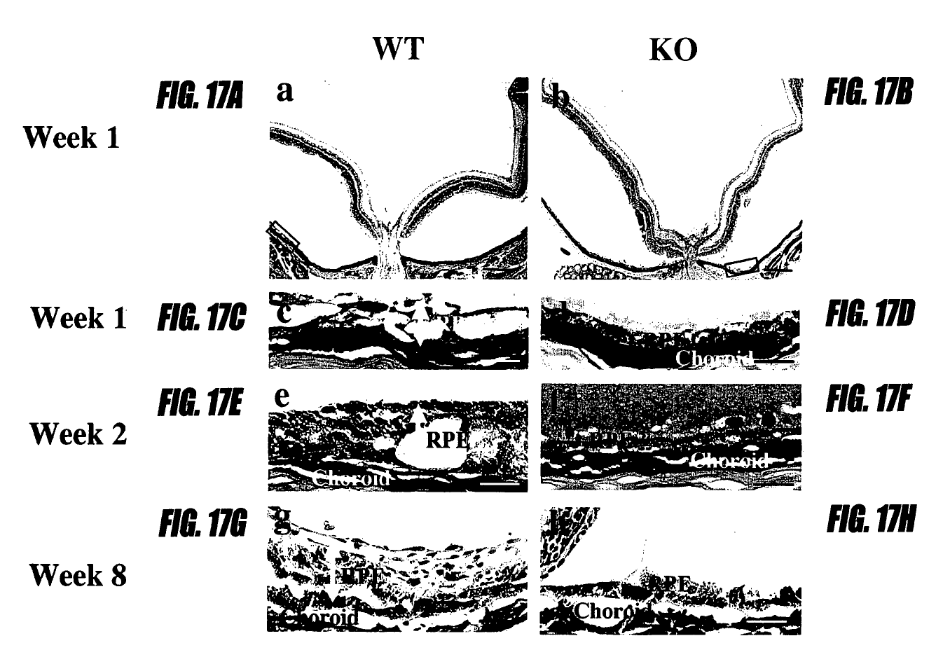

Acceleration of epithelial-mesenchymal transition by exogenous monocytes. Since monocyte influx appeared to play important roles in EMT during UUO, we further investigated a direct interaction of bone-marrow monocytes with renal tubular epithelial cells. Primary culture of wild-type epithelial cells, when co-cultured with wild-type, but not Smad3-null, monocytes for 48 h, expressed a fibroblastic phenotype as characterized by de novo expression of α-SMA with marked reduction of E-cadherin (FIG. 16a, b). Smad3-null epithelial cells showed no phenotypic change in a co-culture with monocytes regardless of their genotypes (FIG. 16c, d).

To examine the effect of the Smad3 genotype of monocytes on the response to UUO in vivo, monocytes were injected into the renal subcapsular space just prior to ligation of the ureter. Wild-type mice transplanted with wild-type monocytes showed a higher number of monocytes infiltrating in the renal cortex (FIG. 16e) as compared with mice transplanted with Smad3-null monocytes (FIG. 16f), indicating that exogenous wild-type, but not Smad3-null, monocytes exhibited increased chemotaxis toward the renal cortex where a level of TGF-β1 is already elevated at day 3 after UUO (FIG. 12h). No influx of transplanted wildtype (FIG. 16g) or Smad3-null monocytes (FIG. 16h) was observed in Smad3-null kidneys, consistent with the lack of elevation of TGF-β in the renal cortex in these kidneys (FIG. 12h). Dual immunofluorescence showed de novo expression of α-SMA with a marked reduction of E-cadherin in wild-type renal cortex transplanted with wild-type monocytes (FIG. 16i). Wild-type mice transplanted with Smad3-null monocytes showed a lesser degree of reduction of E-cadherin expression (FIG. 16j), which was essentially similar to the early phenotypic change seen in renal tubules of wild-type mice at day 3 after UUO; α-SMA was undetectable. Expression of E-cadherin was retained in Smad3 null kidneys transplanted with either wild-type or Smad3-null monocytes (FIG. 16k, l). These findings indicate that exogenous monocytes accelerated the EMT of obstructed kidneys and require Smad3 both for chemotaxis and Smad3-dependent expression of TGF-β1.

Discussion

Epithelial-mesenchymal transition (EMT) of renal tubular epithelial cells has been described in both animal models and TGF-1′-treated cells in culture (Zeisberg, M. et al. 2002 Am J Pathol 160:2001-2008; Iwano, M. et al. 2002 J Clin Invest 100:341-350; Yang, J. et al. 2002 J Am Soc Nephrol 13:2464-2477). Here we demonstrate that Smad3, a signaling intermediate downstream of TGF-β and activin receptors, is essential both for TGF-β 1-induced EMT of cultured renal tubular epithelial cells and for EMT following UUO in vivo. In both of these systems, expression of all markers of EMT is blocked in the absence of Smad3, thus blocking formation of the fibrogenic myofibroblasts from epithelial precursors.

Experiments utilizing mutated forms of the TGF-β type I receptor unable to bind and activate Smad proteins, have clearly shown that the Smad pathway is necessary, but not sufficient for induction of EMT by TGF-β (Itoh, S. et al. 2003 J Biol Chem 278:3751-3761; Yu, L. et al. 2002 EMBO J. 21: 3749-3759), and that other pathways involving phosphatidylinositol 3-kinase, Rho-A and p38MAPK pathways likely are also required (Bakin, A. V. et al. 2000 J Biol Chem 275:36803-36810; Bhowmick, N. A. et al. 2001 Mol Biol Cell 12:27-36; Bhowmick, N. A et al. 2001 J Biol Chem 276:46707-46713). While these experiments do not differentiate between Smad2 and Smad3, we have recently shown that EMT of lens epithelial cells in response to injury in vivo is completely blocked in the absence of Smad3 (EXAMPLE 1). Interesting in this regard, TGF-β-dependent EMT of cardiac endothelial cells required for formation of the endocardial cushions in the atrioventricular canal of the developing heart, is not affected in the Smad3 null mice. This EMT requires expression of the type III TGF-β receptor and may utilize different signaling pathways than those involved in mediating injury-induced EMT (Brown, C. B. et al. 1999 Science 283:2080-2082; Boyer, A. S., & Runyan, R. B. 2001 Dev Dyn 221:454-459).

Although Smad2 and Smad3 are each activated by the TGF-β and activin receptors, they have very different effects on gene transcription (Piek, E. et al. 2001 J Biol Chem 276:19945-19953). Somewhat surprisingly, studies in mouse embryo fibroblasts showed that deletion of Smad3 did not affect endogenous levels of Smad2 or its phosphorylation, and vice versa (Piek, E. et al. 2001 J Biol Chem 276:19945-19953). Thus EMT of renal epithelial cells following UUO is likely independent of Smad2. Smad3 is critical in mediating effects of TGF-β on elaboration of extracellular matrix components including synthesis of collagens by fibroblasts (Verrecchia, F., and Mauviel, A. 2002 J Invest Dermatol 118:211-215) and its loss affords protection from radiation-induced fibrosis (Flanders, K. C. et al. 2002 Am J Pathol 160:1057-1068) and bleomycin-induced pulmonary fibrosis (Zhao, J. et al. 2002 Am J Physiol Lung Cell Mol Physiol 282:L585-L593), presumably by interrupting the pathways necessary for matrix production by fibroblasts. Here we show that Smad3 plays an even more essential role in fibrosis initiated by EMT, since it is also required for generation of the fibrogenic myofibroblasts from epithelial precursors.

The Snail family of zinc-finger transcription factors are strong repressors of transcription of the E-cadherin gene and are implicated in both physiological and pathological EMT (Nieto, M. A. 2002 Nat Rev Mol Cell Biol 3:155-166; Cano, A. et al. 2000 Nature Cell Biol 2:76-82; Hay, E. D. 1995 Acta Anat (Basel) 154:8-20; Carver, E. A. et al. 2001 Mol Cell Biol 21:8184-8188). Recent studies in mouse embryo fibroblasts have identified Snail as an immediate-early gene target of the TGF-β1/Smad3 pathway. Our data that expression of Snail is blocked by neutralizing antibodies to TGF-β in cultured cells in vitro and that EMT is blocked in any condition which interferes with expression of Snail, including loss of Smad3, indicate that it is a critical early response gene in the TGF-β-driven EMT resulting from UUO.

All data obtained following UUO in vivo and from mechanical stress-induced EMT of renal tubular epithelial cells in vitro indicate that EMT is initiated by TGF-β1 produced by the renal tubular cells. Especially convincing are the data showing that a neutralizing antibody to TGF-β1 blocked both elevation of TGF-β1 mRNA and the subsequent EMT of mechanically stressed renal epithelial cells in culture, demonstrating that TGF-β1, and not mechanical force per se, initiates the EMT of the stretched cells. Moreover, the absence of TGF-β1 induction in Smad3-null mice implicates this pathway in injury-induced elaboration of TGF-β1 and amplification through a positive-feedback autoinductive loop, similar to that previously reported in monocytes and fibroblasts (Piek, E. et al. 2001 J Biol Chem 276:19945-19953; Ashcroft, G. S. et al. 1999 Nature Cell Biol 1:260-266). This process may be initiated by activation by mechanical stress of latent forms of TGF-β1 secreted constitutively from renal tubular epithelial cells and sequestered by the matrix. In support of this, endothelial and vascular smooth muscle cells reportedly secrete a higher amount of tissue plasminogen activator (t-PA) in response to shear stress (Diamond, S. L. et al. 1990 J Cell Physiol 143:364-371; Papadaki, M. et al. 1998 Circ Res 16:1027-1034). Thus renal tubular epithelial cells facing high-pressure backflow of urine by UUO may also facilitate activation of latent TGF-β1, through any of many pathways described including proteolytic activation by generation of plasmin from plasminogen by t-PA (Lyons, R. M. et al. 1990 J Cell Biol 110:1361-1367; Sato, Y. et al. 1990 J Cell Biol 111:757-763), or non-proteolytic mechanisms involving thrombospondin-1 (Crawford, S. E. et al. 1998 Cell 26:1159-1170) or αvβ6 integrin (Munger, J. S. et al 1999 Cell 96:319-328; Morris, D. G. et al. 2003 Nature 422:169-173).

TGF-β is one of the most potent cytokines known for chemotaxis of monocytes (Wahl, S. M. et al. 1987 PNAS USA 84:5788-5792; Wiseman, D. M. et al. 1988 Biochem Biophys Res Commun 15:793-800). The significantly reduced levels of monocytes in Smad3-null kidneys following UUO implicates both endogenous TGF-β and the Smad3 pathway in the influx of inflammatory cells in this injury model. Since Smad3-null monocytes also show impaired autoinduction of TGF-β1, the reduced inflammatory influx probably contributes secondarily to the reduced levels of TGF-β1 following UUO. Exogenous monocytes, either co-cultured with renal tubular epithelial cells in vitro or transplanted into the obstructed kidney in the UUO model in vivo, facilitated EMT, providing evidence that the monocyte influx in UUO contributes to the pathogenesis of the developing fibrosis.

In summary, the present results demonstrate that selective ablation of the Smad3 signaling pathway blocks EMT of renal tubular epithelial cells and subsequent pathologic accumulation of matrix proteins while presumably preserving other Smad3-independent TGF-β1 signaling arms. This provides a therapeutic rationale for development of actual inhibitors of Smad3 which should have fewer side effects than either anti-ligand or anti-receptor approaches which block all downstream signaling (Cosgrove, D. et al. 2000 Am J Pathol 157:1649-1659; Peters, H. et al. 1997 Curr Opin Nephrol Hypertens 6:389-393). Our data indicate that selective inhibitors of the Smad3 pathway would prove highly effective in a wide range of fibrotic disorders including not only obstructive nephropathy and chronic interstitial nephritis, but also pulmonary and hepatic fibrosis.

### EXAMPLE 3

### Smad 3 is Required for Subretinal Fibrosis Dependent on Epithelial-Mesenchymal Transition of Retinal Pigment Epithelium Following Retinal Detachment in Mice

Retinal pigment epithelial (RPE) cells undergo epithelial-mesenchymal transition (EMT) following retinal detachment and play a critical role in formation of fibrous tissue on the detached retina and vitreous retraction in a process known as proliferative vitreoretinopathy (PVR). We have developed a mouse model of subretinal fibrosis with implications for PVR in which removal of the crystalline lens and the vitreous and gently touching the retina leads to retinal detachment following which RPE cells undergo EMT and form fibrotic tissue beneath the retina. Transforming growth factory (TGF-β) has long been implicated in EMT of RPEs and the development of PVR. Using mice null for Smad3, a key signaling intermediate downstream of TGF-β and activin receptors, we show that Smad3 is essential for EMT of RPE cells induced by retinal detachment. Specifically, morphological changes of RPE cells to a mesenchymal phenotype characterized by expression of both early and late EMT markers, snail, or α-smooth muscle actin and extracellular matrix components, respectively, were not observed in eyes of Smad3-null mice. We also show that induction of PDGF-BB by Smad3-dependent TGF-β signaling is an important secondary proliferative component of the disease process. The results indicate that blocking the Smad3 pathway would be beneficial in prevention and treatment of PVR.

Introduction

Proliferative vitreoretinopathy (PVR) is one of the major complications of rhegmatogenous retinal detachment surgery and is characterized by the formation of scar-like fibrous tissue containing myofibroblasts derived from transdifferentiated retinal pigment epithelial (RPE) cells, as well as other cell types, such as glial cells, which have entered the vitreous and proliferated on the both anterior and posterior surfaces of the detached retina (Pastor, J. C. et al. 2002 Prog Retin Eye Res 21: 127-144; Casaroli-Marano, R. P. et al. 1999 Invest Ophthalmol Vis Sci 40: 2062-2072). Such fibrocellular tissue may then contract the retina by a cell-mediated process and ultimately reduce the fragility of the detached retina (Sheridan, C. M. et al. 2001 Am J Pathol 159: 1555-1566). PVR can be considered as an excessive wound healing process or a fibrotic response and is the leading cause of failure of retinal detachment surgery with resultant visual loss. RPE cells are activated upon becoming detached from the retina allowing them to disseminate into the subretinal fluid and to enter the vitreous cavity through the retinal tear. In this disease process, RPE cells then transdifferentiate to mesenchymal-like α-smooth muscle actin (αSMA)-positive cells which produce extracellular matrix and contribute to the accumulation of fibrous scar tissue (Casaroli-Marano, R. P. et al. 1999 Invest Ophthalmol Vis Sci 40: 2062-2072; Grisanti, S. and Guidry, C. 1995 Invest Ophthalmol Vis Sci 36: 391-405).

Transdifferentiation of RPE cells to an αSMA-positive phenotype is considered to be an example of EMT, a program of differentiation whereby cells lose their epithelial morphology and expression of epithelial markers such as E-cadherin, and assume a more mesenchymal-like morphology accompanied by expression of markers such as αSMA (Casaroli-Marano, R. P. et al. 1999 Invest Ophthalmol Vis Sci 40:2062-2072; Lee, S. C. et al. 2001 Ophthalmic Res 33:80-86). Although various growth factors, including platelet-derived growth factor (PDGF), hepatocyte growth factor (HGF), connective tissue growth factor (CTGF), transforming growth factor-β (TGF-β), and another member of the TGF-β superfamily, activin, are all reportedly involved in the pathogenesis of PVR (Cassidy, L. et al. 1998 Br J Ophthalmol 82:181-185; Hinton, D. R. et al. 2002 Eye 16:422-428; Kon, C. H. et al. 1999 Invest Ophthalmol Vis Sci 40:705-712; Liang, X. et al. 2000 Chin Med J 113:144-147; Yamamoto, T. et al. 2000 Jpn J Ophthalmol 44:221-226; Bochaton-Piallat, M. L. et al. 2000 Invest Ophthalmol Vis Sci 2000 41:2336-2342), we have focused on TGF-β, because of its correlation with disease severity (Connor, T. B., Jr. et al. 1989 J Clin Invest 83:1661-1666) and its well-described effects on EMT of a variety of epithelial cell types (Bhowmick, N. A. et al. 2001 Mol Biol Cell 12:27-36; Nicolas, F. J. et al. 2003 J Biol Chem 278:3251-3256; Janda, E. et al. 2002 J Cell Biol 156:299-314; Oft, M. et al. 2002 Nat Cell Biol 4:487-494). TGF-β2 is expressed at much higher levels than the other TGF-β isoforms in the vitreous humor (Connor, T. B., Jr. et al. 1989 J Clin Invest 83:1661-1666; Pfeffer, B. A. et al. 1994 Exp Eye Res 59:323-333) and is a likely mediator of EMT in RPE cells in vivo, although the specific signaling pathway involved has not been identified (Lee, S. C. et al. 2001 Ophthalmic Res 33:80-86; Kurosaka, D. et al. 1996 Curr Eye Res 15:1144-1147; Stocks, S. Z. et al. 2001 Clin Experiment Ophthalmol 29:33-37). Moreover, PDGF, TGF-β1 and CTGF are each known to be targets of TGF-β2 signaling (Battegay, E. J. et al. 1990 Cell 63:515-524; Bronzert, D. A. et al. 1990 Mol Endocrinol 4:981-989; Choudhury, P. et al. 1997 Invest Ophthalmol Vis Sci 38:824-833; Leask, A. et al. 2003 J Biol Chem 278:13008-15), indicating that TGF-β2 could orchestrate the secondary effects of these other peptides on EMT and fibrosis in PVR.

Although TGF-β signals are conveyed through multiple common pathways including mitogen-activated protein (MAP) kinases, Smad proteins are unique transducers of signals from the TGF-β family receptor serine-threonine kinases (Piek E. and Roberts A. B. 2001 Adv Cancer Res 83:1-54; Shi Y. & Massague J. 2003 Cell 113:685-700; ten Dijke, P. et al. 2002 J Cell Physiol 191:1-16). Receptor-activated Smad proteins, Smad2 and Smad3, are phosphorylated directly by the TGF-β type I receptor kinase (TβRI), partner with the common mediator, Smad4, and translocate to the nucleus where they play a prominent role in activation of TGF-β-dependent gene targets. While it has been indicated that pathways other than the Smad pathway mediate effects of TGF-β on EMT (Bhowmick, N. A. et al. 2001 Mol Biol Cell 12:27-36; Janda, E. et al. 2002 J Cell Biol 156:299-314; Bakin, A. V. et al. 2000 J Biol Chem 275:36803-36810; Oft, M. et al. 1996 Genes Dev 10:2462-2477), other data indicate that the Smad pathway, together with MAP kinase pathways, is required for EMT of cells by TGF-β (Itoh, S. et al. 2003 J Biol Chem 278:3751-3761; Yu, L. et al. 2002 EMBO J. 21: 3749-3759). Moreover, we have recently used Smad3 null mice (Yang, X. et al. 1999 EMBO J. 18: 1280-1291) to demonstrate that EMT in lens epithelial cells in response to capsular injury and in renal tubular epithelial cells in reponse to injury induced by unilateral ureteral obstruction in vivo is dependent on Smad3 signaling (EXAMPLE 1, EXAMPLE 2). In these studies, we showed that lens epithelial cells and renal tubular epithelial cells of Smad-null mice also lack the ability to undergo EMT in vivo. Based on these findings, we hypothesized that a similar mechanism may be responsible for EMT in RPE cells, and that interference with Smad3 signaling would prevent RPE cells from forming fibrotic tissue characteristic of PVR in a model of retinal detachment.

To test this hypothesis, we have investigated the role of Smad3 signaling in EMT of RPE cells both in vivo and in vitro. We show for the first time that EMT of RPE cells in vivo is completely blocked in Smad3^{ex8/ex8} (KO) mice, as evidenced by the absence of expression of EMT markers including, snail, αSMA, and lumican. Most importantly, the subsequent deposition of collagen and formation of fibrous tissue in the subretinal space seen in eyes of Smad3^{+/+} (WT) littermates post retinal detachment does not occur in the absence of Smad3. To confirm these results and to test whether TGF-β could initiate these same changes in RPE, we show that TGF-β2 can induce EMT in primary porcine RPE cells and a human RPE cell line, ARPE-19 (Dunn, K. C. et al. 1996 Exp Eye Res 62:155-169), in association with Smad phosphorylation in vitro. We show further that TGF-β accelerates migration of ARPE-19 cells in vitro and induces expression of PDGF-BB, which may contribute to the enhanced proliferation of PVR RPE cells and to expression of collagen type I, the major component of ECM of PVR. Taken together, our results indicate that Smad3 signaling is required for EMT in RPE cells following retinal detachment and that inhibition of the Smad3 pathway would be desirable clinically to prevent PVR.

Materials and Methods

All the experimental procedures were approved by Wakayama Medical University, Wakayama, Japan and by the Animal Care and Use Committee of the National Cancer Institute, National Institutes of Health, Bethesda, Md., and conducted in accordance with the ARVO Statement for the Use of Animals in Ophthalmic and Vision Research.

Retinal Detachment Model in Mouse Eyes.

Adult KO and WT mice (4-6 week-old, 37 KO and 43 WT mice) were anesthetized with an intraperitoneal injection of pentobarbital sodium (70 mg/Kg). Procedures to induce retinal detachment in the right eye were performed under a surgical microscope after topical application of mydriatics and oxybuprocaine eyedrops. A linear incision was made in the cornea, keeping the limbus intact. The crystalline lens was carefully removed by using a forceps and the vitreous humor was excised, following which the peripheral retina was carefully broken mechanically with a gentle touch with a silicone rubber needle (Alcon Terry's needle). After adding 1.0% sodium hyaluronate to restore the eye shape, the corneal incision was sutured with 10-0 nylon strings at two sites. Ofloxacine ointment was instilled into the eye and the mice were allowed to heal for specific intervals up to 2 months. Our preliminary experiments showed that the retina becomes detached from the underlying pigment epithelium within one day of the surgical procedure. The left eye served as the uninjured control. Proliferating cells were labeled by an intraperitoneal injection of bromo-deoxyuridine (BrdU) 2 hrs prior to killing the mice by CO₂ asphyxia and cervical dislocation; each eye was enucleated, fixed in 4% paraformaldehyde in 0.1M phosphate buffer and embedded in paraffin. The number of animals examined at each time point was 5/5 (Day 2), 5/5 (Day 5), 7/7 (Week 1), 7/6 (Week 2), 5/4 (Week 3), 5/5 (Week 4) and 9/5 (Week 8) for WT or KO mice, respectively.

Wounding of the RPE Cell Layer in Organ-Cultured Mouse Globes.

Both eyes were enucleated immediately after being sacrificed as described above. Anterior eye segment structures (cornea and lens) and retina were carefully removed from the globe. After the RPE cell layer had been wounded by scraping using a silicone needle, the tissue was cultured in Eagle's medium supplemented with 10% fetal calf serum in the presence or absence of human recombinant TGF-β2 at 10 ng/ml for 48 hr. The tissue was fixed and embedded in paraffin as previously described (Example 1).

Histology and Immunohistochemistry.

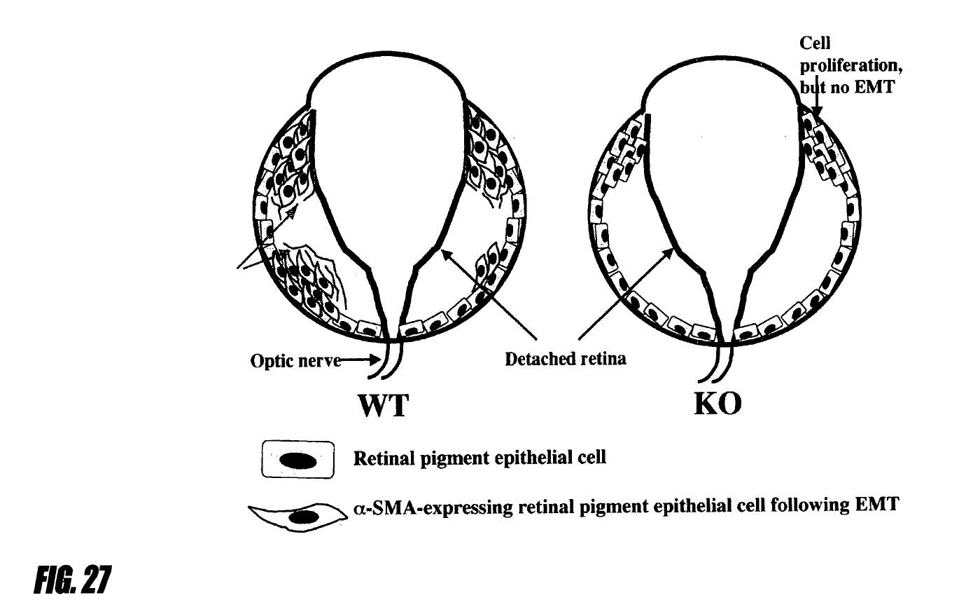

Sections (5 μm) were deparaffinized and stained with hematoxylin and eosin (HE) alone or with polyclonal antibodies against collagen type VI (1:100 dilution in PBS, Southern Biotechnology, Birmingham, Ala.), rabbit polyclonal anti-lumican antibody (10 μg/ml (Saika, S. et al. 2000 J Biol Chem 275:2607-2612)), mouse monoclonal anti-αSMA antibody (1:100 dilution in PBS, NeoMarker, Fremont, Calif., USA), goat polyclonal anti-PDGF-B antibody (1:200 dilution in PBS, Santa Cruz), mouse monoclonal anti-proliferating cell nuclear antigen (PCNA) antibody (1:100 dilution in PBS, Santa Cruz) or with non-immune IgGs (control). After binding of FITC-labeled secondary antibodies (1:100 dilution in PBS, Cappel ICN, Aurora, Ohio, USA), the specimens were observed under fluorescent microscopy followed by mounted with VectaShield with nuclear DAPI staining. For quantitation of PCNA positive cells in vivo, cells counts were restricted to a single section (x40) of the posterior zone of the eye centered around the optic nerve head and representing approximately 90 degrees of the circumference of the eye using computer imaging (see model in FIG. 27). For BrdU immunostaining, monoclonal anti-BrdU antibody (1:10 dilution in PBS; Boehringer Mannheim, Germany) was used. Deparaffinized sections were treated with 2N HCl for 60 min at 37° C. and then processed for indirect immunostaining for BrdU. The antibody complex was visualized with 3,3′-diaminobenzidine as previously reported (Saika, S. et al. 2001 Dev Biol 240:419-432).

In Situ Hybridization for Snail mRNA.

Digoxigenin-labeled riboprobes for mouse snail and were prepared as previously reported using a digoxigenin labeling kit (Roche Diagnostics Corp-Boehringer Mannheim, Indianapolis) (EXAMPLE 1).

EMT and Smad Phosphorylation in Primary Cultures of Porcine RPE Cells or ARPE-19 Cells.

Cultures of primary porcine RPE cells were prepared by aspirating RPE layers from a hemi-sectioned pig eye after removing the retina by using a micropipette, and putting the aspirate in a 30-mm diameter collagen-coated plastic culture dish to allow the epithelial cells to outgrow. After reaching confluence, the cells were trypsinized, suspended in Dulbecco's modified Eagle's essential medium (DMEM) supplemented with 10% fetal calf serum, and seeded on fibronectin-coated chamber slides (Falcon, Becton Dickinson, Lincoln Park, N.J.) in the presence and absence of 10 ng/ml of porcine TGF-β2 (R & D systems Inc., Minneapolis, Minn.). At 24 hr culture intervals the cells were fixed and immunostained for αSMA.

The ARPE-19 human RPE cell line (ATCC # CRL-2302) (Dunn, K. C. et al. 1996 Exp Eye Res 62:155-169) was used to assess effects of endogenous TGF-β2 on expression of αSMA and Smad3 activation as previously described (EXAMPLE 1). Effect of addition of TGF-β2 on ARPE-19 cell migration was examined by using a model of in vitro closure of a monolayer cell sheet as previously reported (Saika, S. et al. 1995 Graefes Arch Clin Exp Ophthalmol 233:347-353).

Production of PDGF TGF-β1 and Collagen Type I in ARPE-19 Cells Treated with TGF-β2.

Expression of PDGF-BB in ARPE-19 cells was assessed by western blot of cell lysates using PDGF-B (H-55, Santa Cruz Biotechnology, Inc.). Enzyme-linked immunosorbent assays (ELISAs) for PDGF-BB, PDGF-AB and TGF-β1 (R & D Systems, Inc., Minneapolis, Minn.) were used to determine the concentration of each peptide in the culture medium. ARPE-19 cells were grown in 6-well plates to near confluence and then cultured in 500 μl of serum-free DMEM/F-12 supplemented with antibiotics in the presence or absence of human recombinant TGF-β2 (1.0 ng/ml R & D Systems, Inc., Minneapolis, Minn.) for 48 or 96 hrs. Nine wells were prepared for each culture condition. Medium was processed for ELISAs for determination of the concentration of PDGF-BB and -AB according to the manufacturers' protocols. Medium (48 hr) was also processed for the determination of TGF-β1 by an ELISA kit (R & D Systems, Minneapolis, Minn.). Type I collagen was quantified in medium and sonicated cell lysates (48 hr) and processed for an ELISA system for type I collagen C-terminal peptide (PIP ELISA kit, Takara, Tokyo, Japan). Confluent cells grown on a chamber slides (Nunc) were immunostained for collagen type I (1:100 dilution in PBS, Southern Biotechnology).

Measurement of Proliferation of ARPE-19 Cells.

Effects of adding TGF-β2, PDGF-BB, and/or anti-PDGF-B-neutralizing antibody on proliferation of ARPE-19 cells were assessed using the MTT assay (TACS MTT Cell Proliferation Assay Kit, Trevigen, Gaithersburg, Md., USA) according to manufacturer's instruction. ARPE 19 cells suspended in DMEM/F-12 supplemented with 15% fetal calf serum (2×10⁴/100 μl/well) were seeded in 96-well culture plates (8 wells for each condition). After 12 hrs cells were changed to 100 μl serum-free culture medium and cultured for an additional 120 hr supplemented with PDGF-BB (5.0 ng/ml) or TGF-β2 (1.0 ng/ml), with either neutralizing anti-PDGF-B antibody (20 μg/ml) or non-immune IgG (20 μg/ml) as control. At the end of each culture interval, MTT reagent (10 μl) was added to each well and the incubation continued for an additional 5 hr at 37° C. Optical density at 570 nm was measured 4 hr after adding the lysis solution contained in the kit.

Results

Smad3 is Required for EMT of RPE Following Retinal Detachment In Vivo.

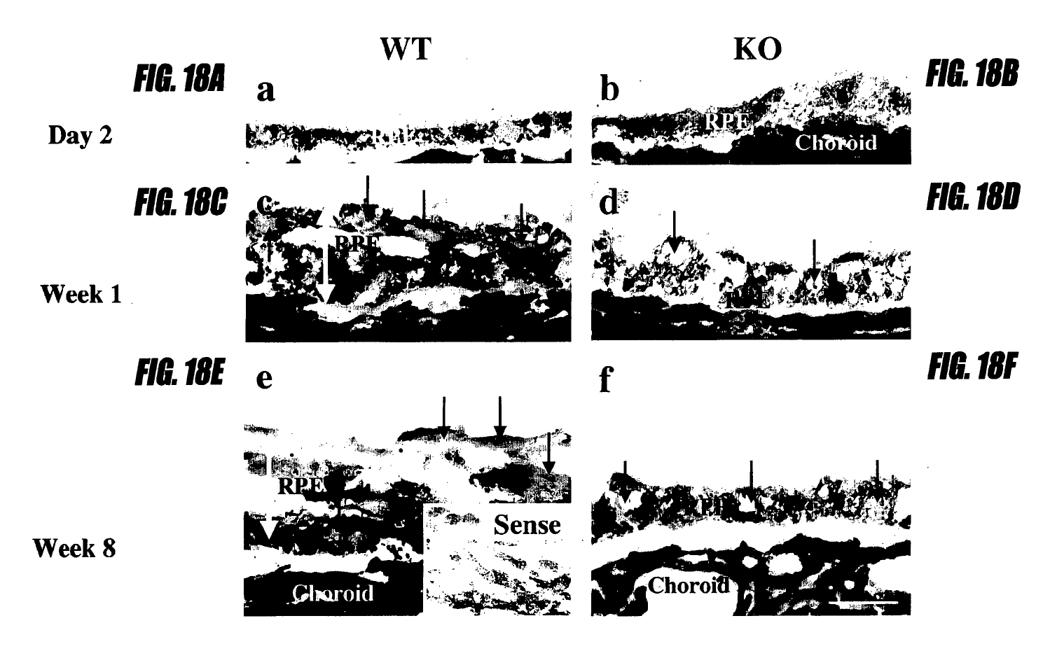

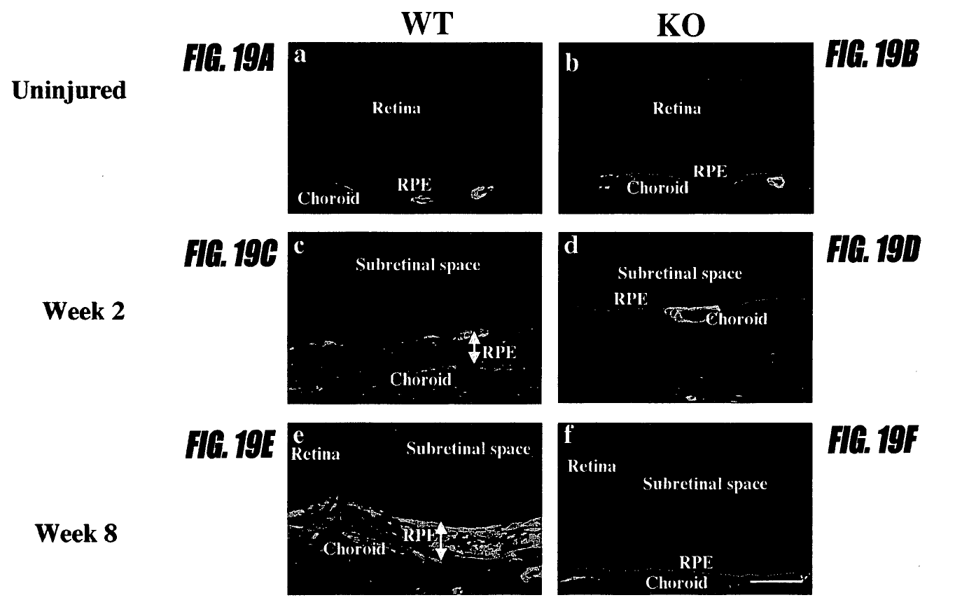

Although several methods to induce retinal detachment in a mouse eye have been described, including transgenic expression of PDGF or VEGF (Ohno-Matsui, K. et al. 2002 Am J Pathol 160:711-719; Seo, M. S. et al. 2000 Am J Pathol 157:995-1005) or injection of substances into the subretinal space (Yeo, J. H. et al. 1986 Arch Ophthalmol 104:417-421; Hisatomi, T. et al. 2001 Am J Pathol 158:1271-1278; Oshima, Y. 2002 Gene Ther 9:1214-1220; Ikuno Y. & Kazlauskas A. 2002 Invest Ophthalmol Vis Sci 43:2406-2411) or into the vitreous (Valeria Canto, S. M. et al. 2002 Exp Eye Res 75:491-504), none of them is appropriate to study EMT of RPE cells, as discussed in detail later. Because of problems with each of these existing models for EMT, we developed a new mouse model of retinal detachment based on a variation of a previously published model (Anderson, D. H. et al. 1986 Invest Ophthalmol Vis Sci 27:168-183). Removal of the crystalline lens and total vitreous humor, followed by a single gentle touch to the peripheral retina by a silicone rubber needle, successfully induced retinal detachment in each mouse without damaging the underlying RPE layers and choroid plexus (FIG. 17a, b).

To address a possible role for Smad3 in the pathogenic response to retinal detachment, we applied this model to both KO mice and WT littermates as controls (Yang, X. et al. 1999 EMBO J. 18:1280-1291). The histology of the uninjured retina and RPE cells in KO and WT eyes was indistinguishable. Injured eyes of KO and WT continued to exhibit a similar histology for the first 2 days post-injury, but differences were clearly apparent at times after Day 5. At Weeks 1 through 8 post-detachment, RPE cells in the posterior pole region became multilayered in WT eyes (FIG. 17a, c, e, g), whereas cells retained their monolayer organization in KO eyes (FIG. 17b, d, f, h). The RPE cells in multilayered regions appeared thinner, and somewhat elongated. While multilayered proliferating cells could be observed in the peripheral area of both WT and KO eyes post-retinal detachment, EMT-like changes were observed only in WT eyes and never in KO eyes throughout the healing period. These histological findings indicated that Smad3 is required for EMT of RPE cells post-retinal detachment. To address this more directly, we analyzed the expression of EMT markers in RPE cells in vivo by immunohistochemistry and in situ hybridization. We focused on the PVR reaction examining the difference of various cellular events, i.e., snail and αSMA expression, accumulation of ECM components and cell proliferation in posterior pole region of the eye.

Expression of Snail in RPE Cells In Vivo is Dependent on Smad3.

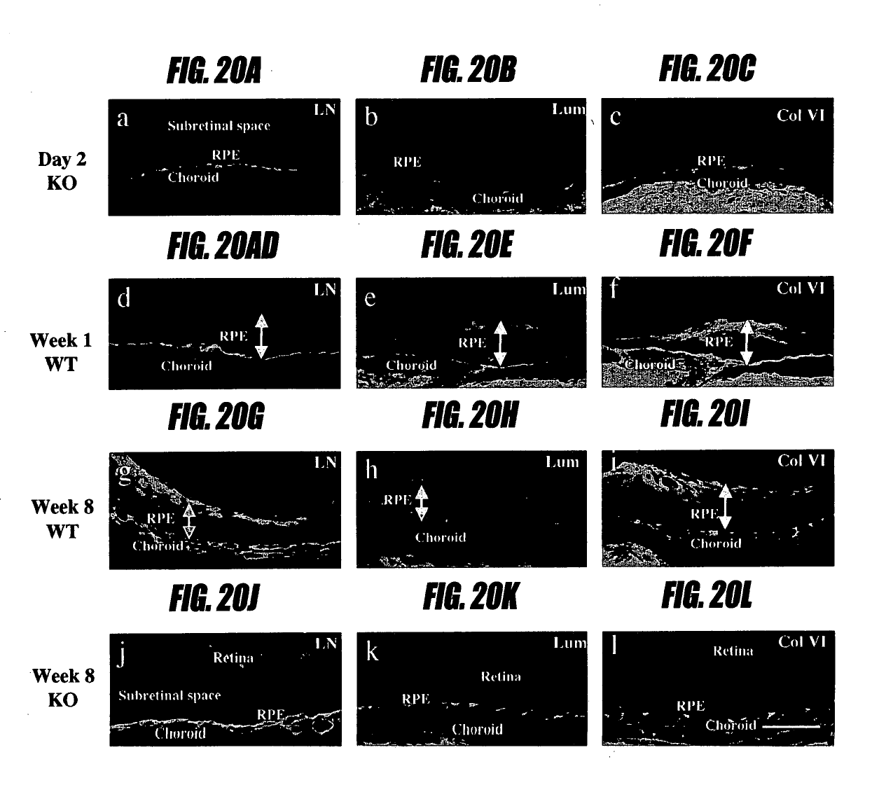

Snail is a member of a family of zinc finger-containing transcriptional repressors increasingly associated with suppression of the epithelial phenotype associated with EMT (Cano, A. et al. 2002 Nat Cell Biol 2:76-83; Carver, E. A. et al. 2001 Mol Cell Biol 21:8184-8188). Snail has also been shown to be an immediate early gene target of the TGF-β/Smad3 pathway in mouse embryo fibroblasts (Yang, Y. C. et al. 2003 PNAS USA 100:10269-10274). To assess whether snail might also be an early marker of Smad3-dependent EMT of RPE cells in vivo, we used in situ hybridization to examine expression of its mRNA in RPE cells of WT and KO mice post-retinal detachment (FIG. 18). Expression of snail mRNA was not detectable in uninjured WT or KO RPE cells, but could be seen in WT RPE cells post-retinal detachment. At Day 2, cells in posterior region were negative for snail mRNA in both WT and KO mice (FIG. 18a, b). However, RPE cells which formed a multi-layered plaque under the detached retina were markedly labeled for snail mRNA in WT mice at Week 1-8 (FIG. 18c, e). Snail mRNA was undetectable in RPE cells of KO mice at all timepoints examined up to Week 8 (FIG. 18d, f). No signal was seen with the sense riboprobe (insert in FIG. 18e).

Expression of other EMT Markers in RPE Cells Following Retinal Detachment is also Dependent on Smad3.

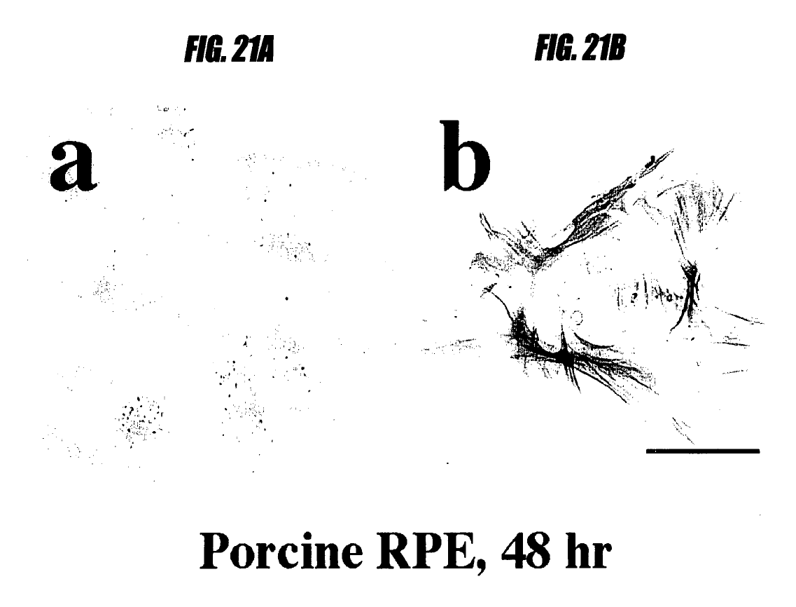

Since both the histology and in situ hybridization for snail were indicative of perturbed EMT in KO RPE cells in response to retinal detachment, we examined whether expression of αSMA, considered to be a hallmark of EMT and of acquisition of a myofibroblast phenotype (Lee, S. C. et al. 2001 Ophthalmic Res 33:80-86; Kurosaka, D. et al. 1996 Curr Eye Res 15:1144-1147; Stocks, S. Z. et al. 2001 Clin Experiment Ophthalmol 29:33-37; Ando, A. et al. 2000 Br J Ophthalmol 84:1306-1311) would also be reduced or absent in KO eyes. Regardless of the genotype, no αSMA protein was detected in uninjured RPE cells or in the first few days post-retinal detachment (FIG. 19a, b). At Weeks 1-3, elongated, pigmented fibroblast-like multilayered RPE cells under the detached retina in WT mice were labeled by the anti-αSMA antibody (FIG. 19c), whereas monolayer RPE cells of KO mice, as well as monolayer cells in WT mice, were not labeled (FIG. 19d). At Weeks 4 and 8, prominent focal fibrous tissue strongly positive for αSMA could be seen in the posterior region of the pigment epithelial layer in WT mice (FIG. 19e), whereas intact RPE cells in the posterior region remained unstained in KO mice (FIG. 19f).

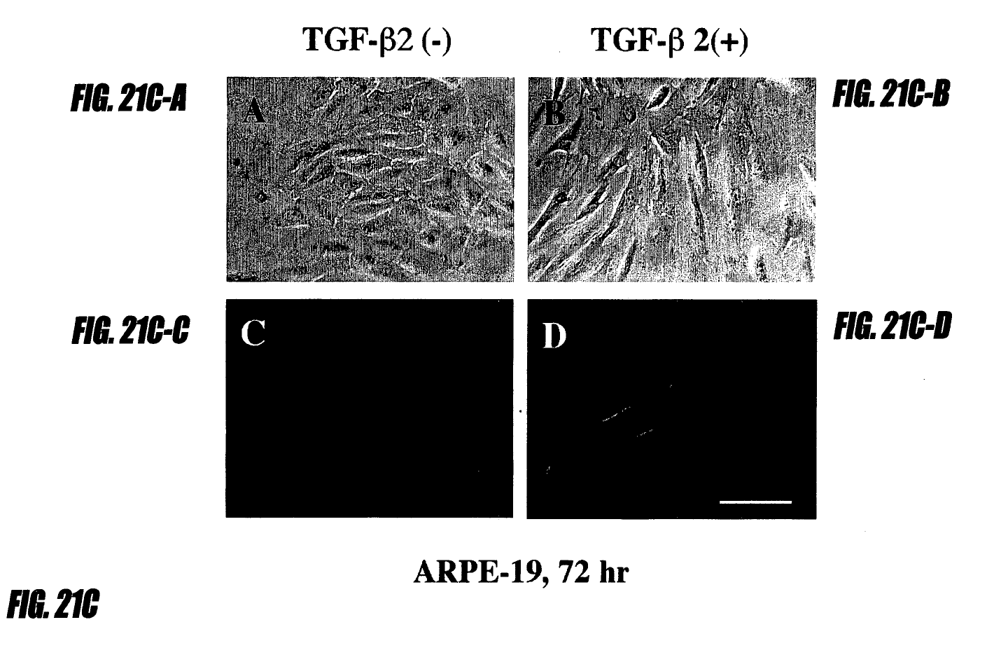

We also examined the expression patterns of lumican, considered to be a late marker for EMT (Saika, S. et al. 2003 Invest Ophthalmol Vis Sci 44:2094-2102), and collagen VI, a component of the pathogenic matrix (Knupp, C. et al. 2002 J Struct Biol 139:181-189). Neither of these markers was detected in RPE cells of an uninjured eye of WT or KO mouse (FIG. 20b, c), whereas the basement membrane protein, laminin, was detected in Bruch's membrane and choroidal vessels (FIG. 20a), and lumican (FIG. 20b), and collagen VI (FIG. 20c), stained strongly in the scleral matrix and weakly in Bruch's membrane. Collagen VI was also detected in choridal blood vessels. One week post-retinal detachment, lumican and collagen VI were expressed in αSMA-positive, pigment-containing, multilayered fibroblast-like cells in WT eyes (FIG. 20e, f), whereas they were not seen in RPEs in KO eyes. At this timepoint immunolocalization of laminin was still restricted to Bruch's membrane in WT eyes (FIG. 20d) and in KO eyes. At Week 8, laminin, lumican and collagen VI were all detected in the fibrous tissue formed under the detached retina in WT eyes (FIG. 20g-i), whereas they were not detected in intact RPE cells in KO mice at this same timepoint (FIG. 20j-l).

TGF-β2 Induces EMT and Smad Phosphorylation of RPE Cells In Vitro.

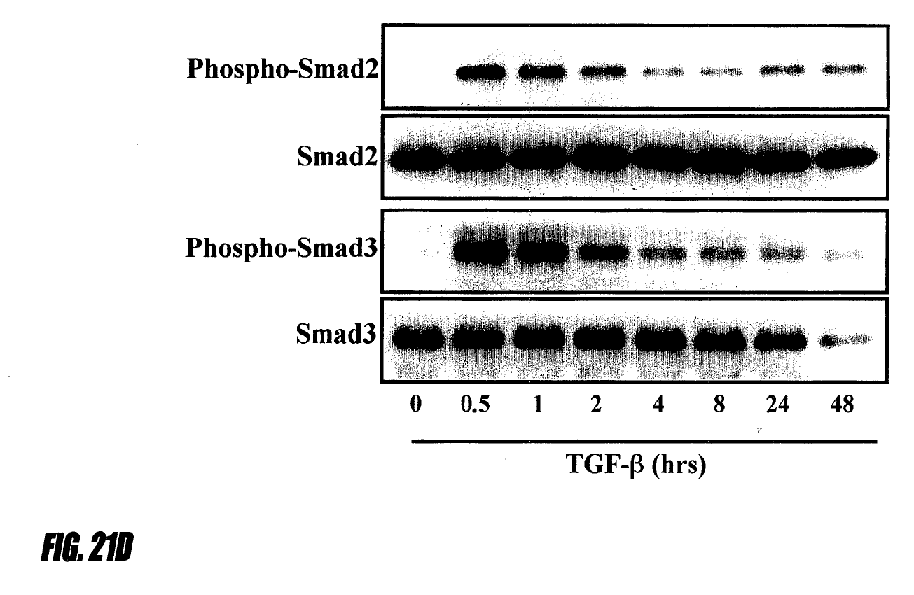

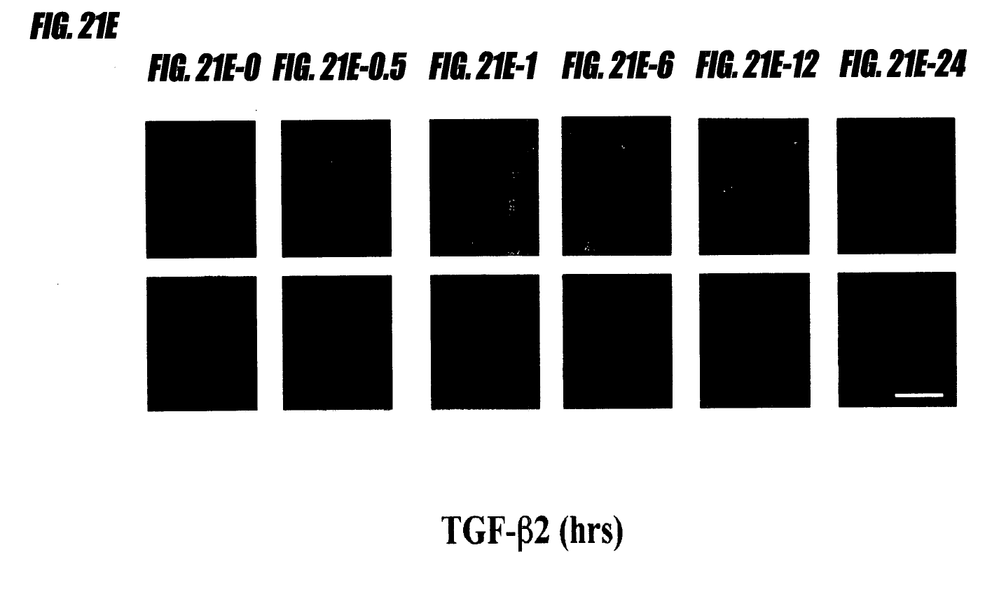

Primary porcine RPE cells express αSMA when cultured in the presence of TGF-β2 (FIG. 21b), but not in its absence (FIG. 21a), in agreement with that shown previously (Lee, S. C. et al. 2001 Ophthalmic Res 33:80-86). Similar to that shown for primary human RPE cells (Stocks, S. Z. et al. 2001 Clin Experiment Ophthalmol 29:33-37), a human RPE cell line, ARPE-19 (Dunn, K. C. et al. 1996 Exp Eye Res 62:155-169), also exhibited a morphological change to a more fibroblastic appearance (FIG. 21cB) and showed clusters of αSMA positive cells after treatment with TGF-β2 for 72 hr (FIG. 21cD) as compared with cells incubated without TGF-β2 (FIG. 21cA, C). Western blotting showed that Smads2/3 were phosphorylated within 30 min after TGF-β2 exposure and remained activated throughout the interval examined up to 48 hrs, although the level of phosphorylation was less than that seen at 30 min-1 hr (FIG. 21d). To ascertain that the phosphorylated Smad3 translocated to the nucleus, indicative of activation of target gene expression, we analyzed these cells by immunofluorescence. Consistent with the kinetics of Smad phosphorylation, nuclear translocation of Smad3 was at its highest level 1 hr after addition of TGF-β2 and was no longer detectable at 24 hr (FIG. 21e). These findings indicate that cultured RPE cells can be induced to undergo EMT coincident with activation of the Smad pathway by TGF-β2.

Wounding of ARPE-19 Cells In Vitro Activates Smad3 Signaling and Cellular Migration.

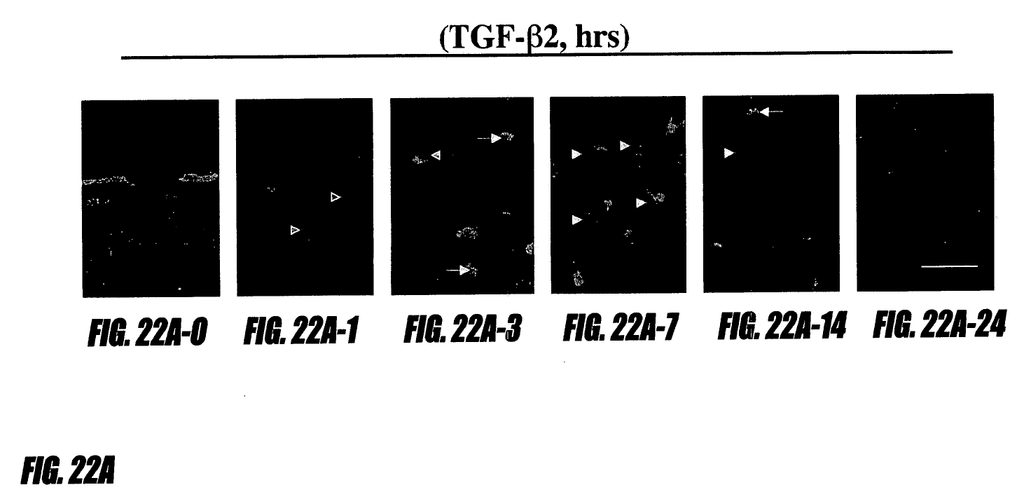

To investigate whether Smad3 might be activated by wounding of RPE, indicative of autocrine TGF-β signaling, we made a defect in a monolayer of ARPE-19 cells (FIG. 22a). Smad3 was detected in the cytoplasm, but not in the nuclei, of cells immediately after wounding (time 0). At 1 hr, faint immunofluorescence for Smad3 was seen in the nuclei of a few cells (arrowheads), which increased to maximal levels by 7 hrs post wounding, a time at which the cells were actively migrating into the wounded space. Similar activation and nuclear translocation of Smad3 has been observed in injured lens epithelial cells (Saika, S. et al. 2002 Br J Ophthalmol 86:1428-1433). Addition of exogenous TGF-β2 accelerated cell migration, resulting in closure of the defect by 12 hr (FIG. 22b, Panel D), compared to 24 hrs for the untreated culture (FIG. 22b, Panel E).

Injury-Induced EMT of RPE in Organ-Cultured Mouse Globes Requires Smad3.

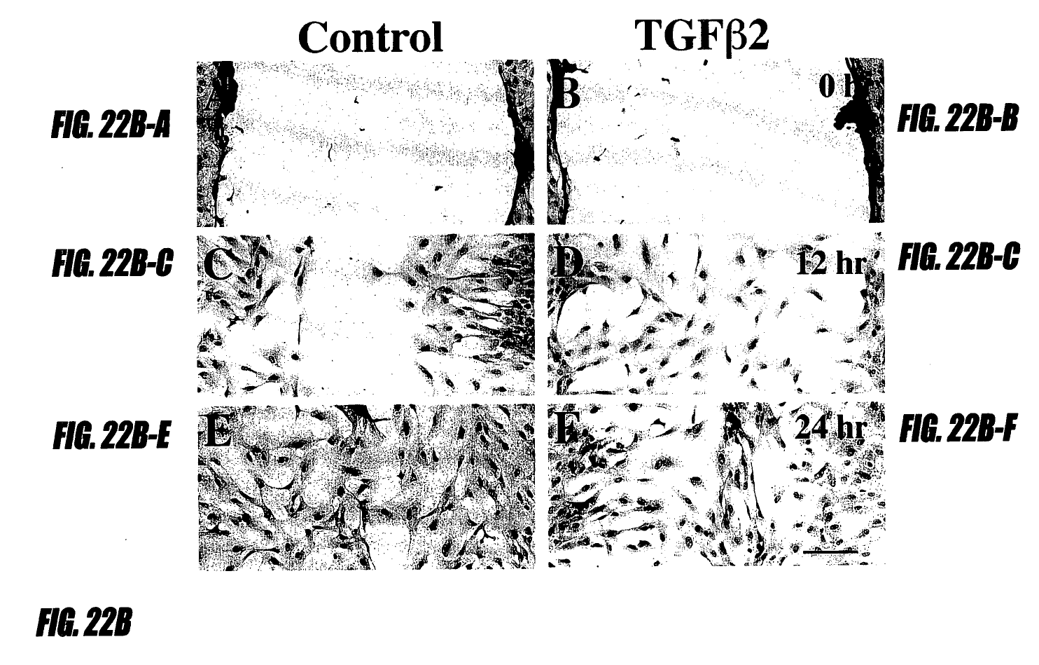

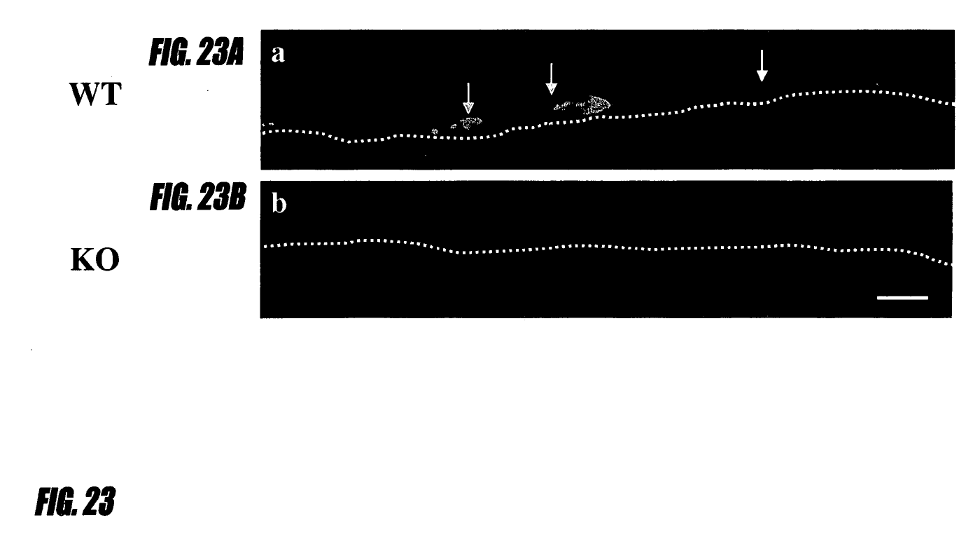

To confirm the Smad3 dependence of EMT of RPE in response to injury, we employed another model involving organ-cultured posterior segments of mouse eyes scrape-wounded in vitro and treated with TGF-β2 to mimic exposure to vitreous. Wounded WT pigment epithelium expresses αSMA faintly at 24 hr and then clearly at 48 hr after exposure to TGF-β2 (FIG. 23a). Wounded WT posterior segments cultured in the absence of TGF-β2 and wounded KO posterior segments cultured in the presence of TGF-β2 were negative for αSMA at this same timepoint (FIG. 23b).

Proliferating RPE Cells Express PDGF-BB in PVR Tissue Post-Retinal Detachment In Vivo.

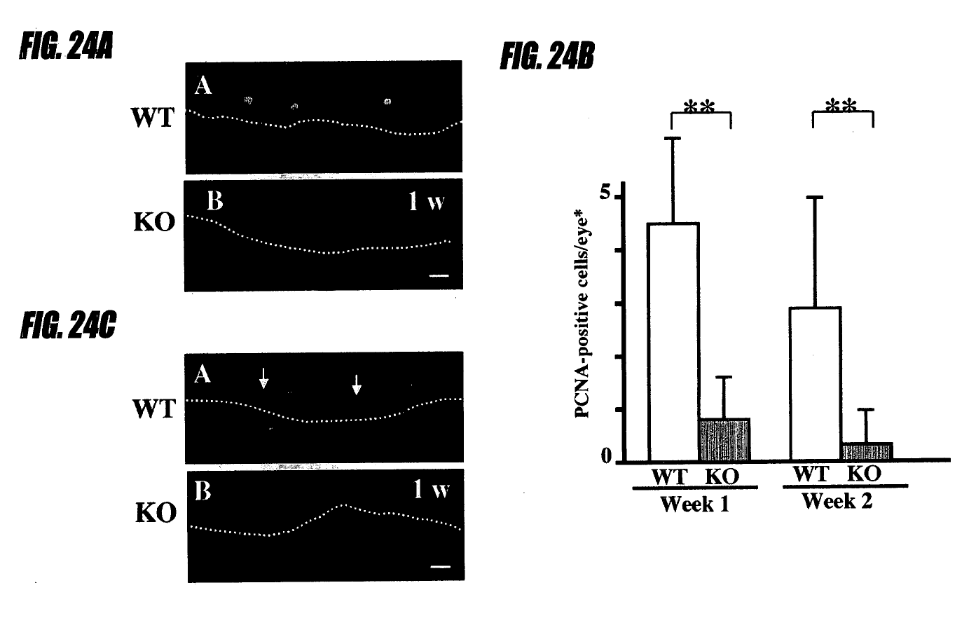

Histologically, RPE cells undergoing EMT in vivo in response to retinal detachment had a multi-layered appearance indicative of proliferation, despite the fact that TGF-β2, thought to be a key player in the pathogenesis of PVR, is known to inhibit the proliferation of RPE cells (Lee, S. C. et al. 2001 Ophthalmic Res 33:80-86). To identify and quantify proliferating cells in the RPE multilayer formed under the detached retina, tissues were immunostained with anti-PCNA antibody (FIG. 24a) or with anti-BrdU antibody. PCNA-positive RPE cells were observed in cell multilayers formed in WT mice at Week 1 (FIG. 24aA), but not at weeks 2-8. No PCNA-positive cells were detected in the RPE cell layer immediately after induction of retinal detachment of a WT mouse or in RPE cells of KO mice at any timepoint (FIG. 24aB, at Week 1). These results are quantified in FIG. 24b.

Because PDGF-BB, a potent mitogen, has also been implicated in the pathogenesis of PVR both in mice (Seo, M. S. et al. 2000 Am J Pathol 157:995-1005; Yeo, J. H. et al. 1986 Arch Ophthalmol 104:417-421) and in humans (Cassidy, L. et al. 1998 Br J Ophthalmol 82:181-185; Liang, X. et al. 2000 Chin Med J 113:144-147), we examined if PDGF-BB could be detected in the RPE compartment post-retinal detachment. Newly formed PVR tissue in WT mice containing fibroblast-like RPE cells were labeled with anti-PDGF-BB antibody at all times examined after Week 1 post-retinal detachment (FIG. 24cA), while RPE cells in KO mice neither formed multilayers nor expressed PDGF-BB (FIG. 24cB). PDGF was considered to accumulate in matrix of PVR tissue because collagen types are known to be ligands for PDGF in tissue (Somasundaram, R. and Schuppan, D. 1996 J Biol Chem 271:26884-26891).

Effects of TGF-β2 and PDGF on Cell Proliferation of ARPE-19 Cells.

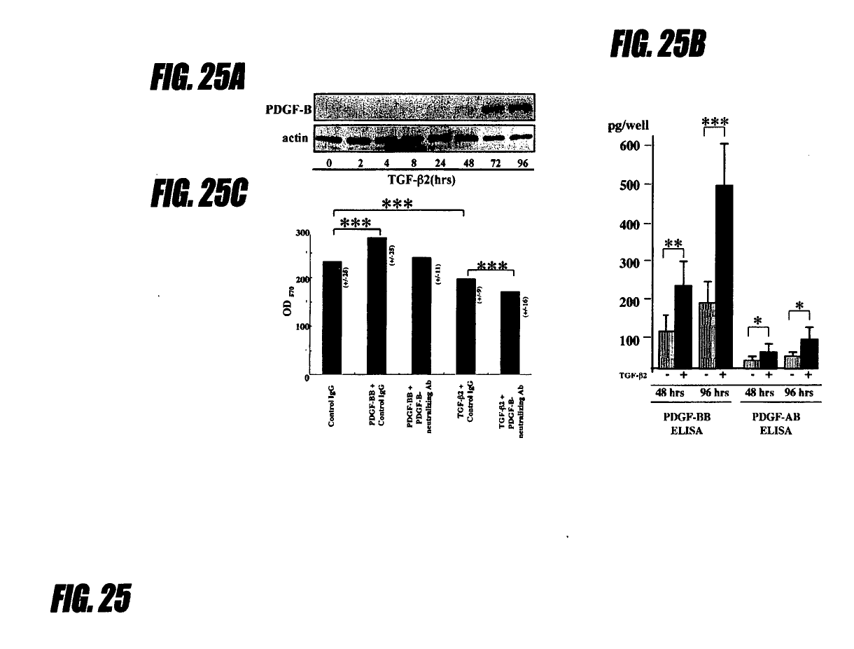

Since we showed that PDGF-BB was expressed in PVR tissue of WT mouse eyes in areas of cell proliferation, but to a significantly lesser extent in RPE in KO eyes following retinal detachment, we examined if exogenous TGF-β2 up-regulates PDGF expression in ARPE-19 cells. Western blotting showed a time-dependent up-regulation of PDGF-B protein expression beginning about 48-72 hrs after addition of TGF-β2 (FIG. 25a). This was confirmed by quantifying PDGF-BB and PDGF-AB in media of confluent cultures by an ELISA. Treatment of cells with TGF-β2 increased the amounts of PDGF-BB protein secreted into the culture medium over 2-fold above that of control cultures (FIG. 25b). The concentration of PDGF-BB reached approximately 500 pg/ml in cultures treated with TGF-β2 for 96 hrs. Although the total amount of PDGF-AB secreted by ARPE-19 cells was much less as compared than that of PDGF-BB, its production was also significantly increased in the presence of TGF-β2. The crystal violet color reaction was used to show that there were no significant differences in the cell numbers among these cultures.

Since RPE Cells Proliferate to Form Subretinal PVR-Like Tissue Post-Retinal detachment in vivo, and since TGF-β2 inhibits the growth of most epithelial cells, we examined effects of TGF-β2 (1 ng/ml), PDGF-BB (5 ng/ml), and TGF-β2 plus anti-PDGF-B antibody (20 μg/ml) on cell proliferation in sparse, growing, cultures of ARPE-19 cells (FIG. 25c). The efficacy of the anti-PDGF-B antibody was confirmed in cultures containing both PDGF-BB and the neutralizing antibody in a 120 hr culture. As expected, PDGF-BB enhanced and TGF-β2 inhibited the growth of the cells in a time-dependent manner as measured by the MTT assay. Addition of a PDGF-B neutralizing antibody to TGF-β2 culture resulted in further suppression of cell proliferation, indicating that the time-dependent accumulation of endogenous PDGF-BB counteracts the growth inhibitory effects of exogenous TGF-β2.

ARPE-19 Cells Express TGF-β1 and Collagen Type I when Treated with TGF-β2.

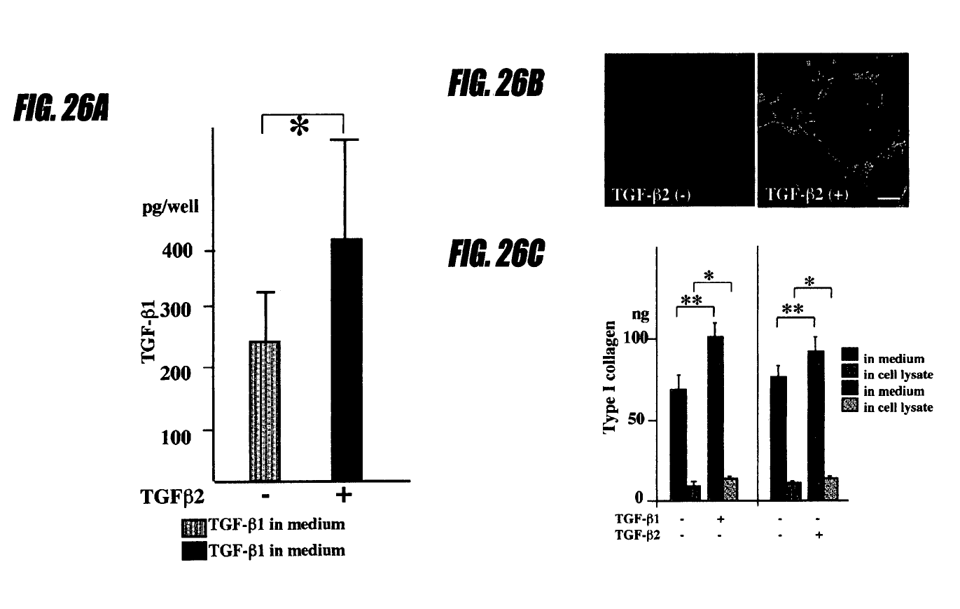

Treatment of ARPE-19 cells for 48 hr with exogenous TGF-β2 caused a significant increase in expression of TGF-β1 (FIG. 26a). TGF-β-treated cells also show increased deposition of type I collagen as shown by both immunofluorescence and by quantifying in culture medium and cell lysate using an ELISA assay (FIGS. 26b and c), indicating that the Smad3 dependent deposition of collagen in the sub-retinal space post retinal detachment in vivo is likely also dependent on TGF-β2.

Discussion

In the present study, we have described a new model of retinal detachment in the mouse eye resulting in separation of the neural retina from the underlying pigment epithelium. The exposure of the RPE cells to subretinal fluid causes them to transition to a myofibroblast phenotype (EMT) as indicated by their de novo expression of αSMA and other markers, leading ultimately to fibrosis, and thus modeling the key features of the human disease PVR (Casaroli-Marano, R. P. et al. 1999 Invest Ophthalmol Vis Sci 40:2062-2072). Most importantly, we have shown that this process does not occur in the absence of Smad3, implicating activation of this pathway by TGF-β2, or possibly another member of the TGF-β superfamily, activin, which shares the same downstream mediators, in the pathogenesis of PVR (FIG. 27). This is consistent with our earlier report showing a correlation between levels of TGF-β2 in the vitreous and the degree of severity of PVR (Connor, T. B., Jr. et al. 1989 J Clin Invest 83:1661-1666), and with identification of actvin both in PVR and as a product of RPE cells in vitro (Yamamoto, T. et al. 2000 Jpn J Ophthalmol 44:221-226; Jaffe G. J. et al. 1994 Invest Ophthalmol Vis Sci 35:2924-2931).

Several models for retinal detachment have been used to identify pathogenic mechanisms that contribute to PVR. One commonly used model involving injection of genetically engineered or normal conjunctival fibroblasts into the vitreous has demonstrated key roles for PDGF and the PDGFRalpha (Yeo, J. H. et al. 1986 Arch Ophthalmol 104:417-421; Hisatomi, T. et al. 2001 Am J Pathol 158:1271-1278; Oshima, Y. et al. 2002 Gene Ther 9:1214-1220). However, this model cannot be used to study earlier stages of PVR dependent on EMT of RPE cells. Supporting this, we have shown that differentiation of fibroblasts to αSMA-positive myofibroblasts induced by TGF-β is independent of Smad3 (Flanders, K. C. et al. 2003 Am J Pathol in press), whereas injury-induced differentiation of epithelial cells to mesenchymal-like cells expressing αSMA, as evidenced by EMT of lens epithelium or renal tubular epithelium, is clearly Smad3-dependent (EXAMPLE 1; EXAMPLE 2). Other models of PVR are based on transgenic mice in which overexpression of various growth factors in the photoreceptor cells leads to traction detachment of the retina (Ohno-Matsui, K. et al. 2002 Am J Pathol 160:711-719; Seo, M. S. et al. 2000 Am J Pathol 157:995-1005). These approaches alter the cytokine milieu and potentially alter processes such as EMT of RPE cells. Intravitreal injection of dispase has also been used to induce PVR, but it might stimulate retinal glial cells to migrate toward the subretinal space (Valeria Canto, S. M. et al. 2002 Exp Eye Res 75:491-504). So while each of these models provides insights into aspects of PVR, most of them represent an unlikely extreme and do not mimic the natural pattern of cytokine expression during the pathogenesis of PVR. The model we have used here has been modified from one originally described elsewhere (Anderson, D. H. et al. 1986 Invest Ophthalmol Vis Sci 27:168-183). Our model (FIG. 27) supports analysis of both the initial events involved in activation and mesenchymal transition of RPE cells and later events leading to accumulation of ECM.

Using this model, we have shown that following retinal detachment in vivo, WT RPE cells exhibit a morphological transdifferentiation to fibroblastic cells, while retaining expression of pigment in the cytoplasm. Such histological findings prompted us to hypothesize that these RPE cells are undergoing EMT. Indeed, the cells display all of the classic features of EMT including expression of the early marker snail (Cano, A. et al. 2002 Nat Cell Biol 2:76-83; Carver, E. A. et al. 2001 Mol Cell Biol 21:8184-8188), and of later markers αSMA, the hallmark of myofibroblasts, and lumican and collagen VI, components of the pathologic ECM (Knupp, C. et al. 2002 J Struct Biol 139:181-189). None of these markers is detectable in RPE cells in eyes of Smad3 null mice post-detachment. The results obtained in this model of retinal detachment in vivo are supported further by in vitro data using not only a human RPE cell line, ARPE-19 (Dunn, K. C. et al. 1996 Exp Eye Res 62:155-169), but also primary porcine RPE cells and organ culture of injured WT and KO murine pigment epithelium. These studies clearly show that TGF-β2 can induce similar changes in these cells in vitro as seen post-retinal detachment in vivo. Thus treatment of ARPE-19 human RPE cells with TGF-β2 activated Smad signaling and induced expression of αSMA and collagen. Together with the in vivo data and organ-culture experiments using eyes from WT or KO mice, these data implicate the Smad3 pathway in injury-induced EMT in RPE cells, and ultimately in processes leading to fibrosis and traction detachment of the retina characteristic of PVR.

There is controversy in the literature concerning the origin of the αSMA positive cells in the scar-like epiretinal membranes characteristic of PVR. We and others have clearly demonstrated that primary RPE cells in culture, ARPE-19 cells, and RPE cells in injured retinas in organ culture can express αSMA associated with an acquisition of a fibroblastic morphology and a migratory, contractile phenotype, which is enhanced by treatment with exogenous PDGF or TGF-β (Lee, S. C. et al. 2001 Ophthalmic Res 33:80-86; Kurosaka, D. et al. 1996 Curr Eye Res 15:1144-1147; Stocks, S. Z. et al. 2001 Clin Experiment Ophthalmol 29:33-37; Choudhury, P. et al. 1997 Invest Ophthalmol Vis Sci 38:824-833; Campochiaro P. A. and Glaser B. M. 1985 Arch Ophthalmol 103:576-579; Carrington, L. et al. 2000 Invest Ophthalmol Vis Sci 41:1210-1216). Studies of intermediate filament proteins in vivo indicate that cells in epiretinal membranes of PVR also represent dedifferentiated RPE cells which express αSMA and have acquired a mesenchymal migratory, phenotype (Casaroli-Marano, R. P. et al. 1999 Invest Ophthalmol Vis Sci 40:2062-2072). Alternatively, it has been indicated that these myofibroblasts might arise from astrocytes or even from pericytes of the retinal vasculature (Bochaton-Piallat, M. L. et al. 2000 Invest Ophthalmol Vis Sci 2000 41:2336-2342). Our observations, based on both in vitro and in vivo models, are in agreement with RPE cells being one of the sources of αSMA positive myofibroblasts and demonstrate that their differentiation is dependent on Smad3.

Recently we have shown a similar requirement for Smad3 in injury-induced EMT of both lens epithelial cells and kidney tubular epithelial cells using mouse models of lens anterior capsular opacification and obstructive kidney disease, respectively (EXAMPLE 1; EXAMPLE 2). In the ocular lens, similar to what we report here in the pigment epithelium, the generation of cells expressing snail, lumican, and αSMA post-injury has a stringent dependence on Smad3 (EXAMPLE 1). In unilateral ureteral obstruction, influx of macrophages into the cortex of the obstructed kidney, and transition of the tubular epithelial cells to myofibroblasts as evidenced by loss of expression of E-cadherin and de novo expression of snail, αSMA and type I collagen are each Smad3 dependent (EXAMPLE 2). In each of these models, in vitro studies with lens epithelial cells and primary kidney tubular epithelial cells demonstrated that the effects of injury in vivo are recapitulated by treatment of cells with TGF-β in vitro, indicating that injury induces activation of TGF-β that then drives the fibrotic sequelae dependent on signaling through the Smad3 pathway. This implication of the Smad3 pathway as essential for injury-induced EMT is also supported by studies in NMuMg murine mammary epithelial cells, where use of a mutant TβRI unable to bind or activate Smad2/3 but still competent to signal through MAPK pathways, has shown that the Smad pathway is necessary but possibly not sufficient to effect EMT driven by TGF-β (Itoh, S. et al. 2003 J Biol Chem 278:3751-3761; Yu, L. et al. 2002 EMBO J. 21:3749-3759). Some of the cooperating pathways are likely to include phosphatidylinositol 3-kinase, RhoA, and MAPK pathways (Bhowmick, N. A. et al. 2001 Mol Biol Cell 12:27-36; Janda, E. et al. 2002 J Cell Biol 156:299-314; Bakin, A. V. et al. 2000 J Biol Chem 275:36803-36810; Oft, M. et al. 1996 Genes Dev 10:2462-2477).

While our data clearly implicate TGF-β/Smad3 signaling in EMT of RPE cells, and likely also in their migration post-injury, thought to contribute to the contractile properties of fibrocellular membranes (Sheridan, C. M. et al. 2001 Am J Pathol 159:1555-1566), cytokines other than TGF-β probably contribute to the proliferative aspects of the disease. TGF-β is inhibitory to growth of RPE cells (Lee, S. C. et al. 2001 Ophthalmic Res 33:80-86) and on epithelia in general (Roberts A. B. and Sporn M. B. 1990 Handbook of Experimental Pharmacology. Peptide Growth Factors and Their Receptors. Eds. Sporn M. B. & Roberts A. B. New York, Springer-Verlag, p. 419-472). Many lines of evidence indicate that PDGF, a mitogenic growth factor, contributes to the proliferation of RPE cells post-injury. PDGF, like TGF-β2, has been implicated in PVR in vivo (Cassidy, L. et al. 1998 Br J Ophthalmol 82:181-185; Liang, X. et al. 2000 Chin Med J 113:144-147; Seo, M. S. et al. 2000 Am J Pathol 157:995-1005) and in accelerating transition of RPE cells in vitro (Ando, A. et al. 2000 Br J Ophthalmol 84:1306-1311). Moreover, an in vitro model of the later contractile stages of PVR has shown that PDGF mediates the contractile effects of TGF-β on RPE cells (Choudhury, P. et al. 1997 Invest Ophthalmol Vis Sci 38:824-833; Carrington, L. et al. 2000 Invest Ophthalmol Vis Sci 41:1210-1216). We have shown that TGF-β can induce synthesis and secretion of PDGF-BB and -AB in ARPE-19 cells, similar to its ability to induce synthesis of PDGF and activate PDGF receptors in a variety of other cells (Battegay, E. J. et al. 1990 Cell 63:515-524; Bronzert, D. A. et al. 1990 Mol Endocrinol 4:981-989; Sintich, S. M. et al. 1999 Endocrinology 140:3411-3415). Importantly, this induction of PDGF by TGF-β has recently been demonstrated to be Smad3/4 dependent (Taylor L. M. & Khachigian, L. M. 2000 J Biol Chem 275:16709-16716). The absence of PDGF-BB expression in the KO RPE cells post-retinal detachment indicates that its expression in vivo might be dependent on TGF-β and not a direct result of the injury. This is consistent with the delayed expression of PDGF both in vivo and in vitro and the demonstration that antibodies to PDGF-BB enhanced the growth suppressive effects of TGF-β on ARPE-19 (FIG. 25c). These data strengthen the argument that antagonists of Smad3 would be able to block the fibrogenic traction detachment of the retina not only at the level of TGF-β-mediated mesenchymal transition of RPE cells but also by blocking of expression of another key mediator of the disease process, PDGF.

It is still unclear whether TGF-β1 is also important in PVR. We did not observe significant immunostaining for TGF-β1 in cell multilayers formed beneath the detached retina in WT type mice, although we have shown that addition of exogenous TGF-β2 up-regulates production of TGF-β1 in ARPE-19 cells.

Although it has been shown that PVR induced in pigmented rabbits by intravitreal injection of rabbit conjunctival fibroblasts is efficiently treated by intravitreal application of an adenoviral vector encoding a soluble type II TGF-β receptor, which sequesters TGF-β1/3, but not TGF-β2 (Oshima, Y. 2002 Gene Ther 9:1214-1220), it must again be considered that pathogenic mechanisms in this PVR model of injection of fibroblasts into the eye are probably distinct from those of PVR caused by EMT of RPE cells.

In summary, the central role of Smad3 not only in EMT of RPE cells, but also in their expression of PDGF and the elaboration of ECM by proliferating mesenchymal-like cells produced through EMT of RPE cells indicates that it should be an important new target for design of therapeutics against PVR. Interfering with Smad3 signaling is envisioned to have effective clinical application in treatment of this devastating disease that can lead to blindness.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A method of identifying compounds for amelioration of Smad3 mediated epithelial to mesenchymal transition (EMT) comprising the steps of:
a) administering a test Smad3 inhibitor to a cell-based or animal model-based system; and
b) measuring the effect on Smad3 mediated EMT, wherein a compound is selected as a candidate on the basis of amelioration of Smad3 mediated EMT.

2. The method of claim 1 wherein the system is the cell-based system.

3. The method of claim 2 wherein the cell-based system is lens epithelial cells.

4. The method of claim 2 wherein the cell-based system is renal epithelial cells.

5. The method of claim 2 wherein the cell-based system is retinal epithelial cells.

6. The method of claim 1 wherein the test Smad3 inhibitor is a member of the group consisting of peptides and analogues thereof, proteins, fusion proteins, carbohydrates, lipids, nucleic acid sequences such as aptamers, antibodies (including anti-idiotypic antibodies) and fragments thereof, small organic compounds (e.g., peptidomimetics) and inorganic compounds, Smad3 antisense, Smad3 ribozymes, and Smad3 interfering RNAs.

7. The method of claim 6 wherein the test Smad3 inhibitor is a small organic compound.

8. The method of claim 1 wherein the effect on Smad3 mediated EMT is measured by detecting a change in the expression of the Smad3 gene, a change in the activity of the Smad3 gene product, or a change in Smad3 regulated signal transduction.

9. The method of claim 8 in which expression of the Smad3 gene is detected by measuring mRNA transcripts of the Smad3 gene.

10. The method of claim 8 in which expression of the Smad3 gene is detected by measuring Smad3 protein.

11. The method of claim 8 in which Smad3 regulated signal transduction is detected by measuring by amino acid phosphorylation of a host cell protein.

12. The method of claim 8 in which activity of the Smad3 gene product is detected by measuring generation of fibrogenic myofibroblasts from epithelial precursors.

13. The method of claim 8 in which activity of the Smad3 gene product is detected by measuring expression of an early EMT marker, optionally selected from snail and its homolog slug, SIP1, and E-cadherin.

14. The method of claim 8 in which activity of the Smad3 gene product is detected by measuring expression of a late EMT marker, optionally selected from αSMA, lumican, collagen and other extracellular matrix proteins.

15. The method of claim 8 in which activity of the Smad3 gene product is detected by measuring PDGF.

16. The method of claim 8 in which Smad3 regulated signal transduction is mediated by ligands selected from activins, AMH, BMPs, and TGF-βs; type II receptors selected from ActR-II, ActR-IIB, AMHR-II, BMPR-II, and TβR-II; Type I receptors selected from ALK117; R-Smads selected from Smad 1, 2, 5, and 8; I-Smads selected from Smad 6 and 7; co-Smads selected from Smad 4α and β; scaffolding proteins selected from Axil, Axin, Caveolin-1, Dab-2, Hrs/Hgs, SARA, SNIX, Strap, TLP, and TRAP-1; cytoskeletal components selected from filamin-1 and tubulin; nuclear transporters selected from CRM1, Importinβ, and Ran GTPase; transcriptional regulators selected from AR, ATF-2, BF-1, E1A, ER, Evi-1, FAST/FosH1, c-Fos, Gli3, GR, c-Jun, JunB, JunD, HNF4, LEF/TCF, MEF2, Menin, Milk, Mixer, Miz-1, MyoD, OAZ, p52, PEBP2/CBFA/AML, pX, SNIP1, Spl, Sp3, Tax1, TFE3, and VDR; transcriptional co-activators selected from MSG1, p300/CBP, and P/CAF; transcriptional repressors selected from Hoxa-9 and Hoxc-8, and/or transcriptional co-repressors selected from HDACs, Ski, SnoN, and TGIF.

17. The method of claim 1 further comprising combining the compound so identified in admixture with a carrier to form a composition.

18. The composition produced by the method of claim 17.

19. A method of ameliorating Smad3 mediated epithelial to mesenchymal transition (EMT) comprising administering the composition of claim 18 to a patient in need thereof to ameliorate said Smad3 mediated EMT.

20. The method of claim 19 wherein said Smad3 mediated EMT is selected from EMT of lens epithelium, EMT of renal epithelium, and EMT of retinal epithelium.
